(19) United States
(12) Patent Application Publication
(10) Pub. No.: US 2023/0382900 A1
(43) Pub. Date: Nov. 30, 2023

(54) INHIBITORS OF BTK

(71) Applicant: Newave Pharmaceutical Inc., Pleasanton, CA (US)

(72) Inventor: Yi Chen, Pleasanton, CA (US)

(21) Appl. No.: 18/033,481

(22) PCT Filed: Oct. 29, 2021
(86) PCT No.: PCT/US2021/057187

§ 371 Date: Apr. 24, 2023

Related U.S. Application Data
(60) Provisional application No. 63/233,263, filed on Aug. 14, 2021.
(60) Provisional application No. 63/108,094, filed on Oct. 30, 2020.

(51) Int. Cl. C07D 405/14; C07D 487/04; A61P 35/00

(52) U.S. Cl. CPC C07D 405/14; C07D 487/04; A61P 35/00

(57) ABSTRACT

The disclosure includes compounds of Formula (I), or Formula (I) wherein Q₀, Q₂, Q₃, Q₄, Z, i, j, n, Warhead, R₀, R₃, R₄, R₅, R₆, and R₇, are defined herein. Also disclosed is a method for treating a neoplastic disease, autoimmune disease, and inflammatory disorder with these compounds.

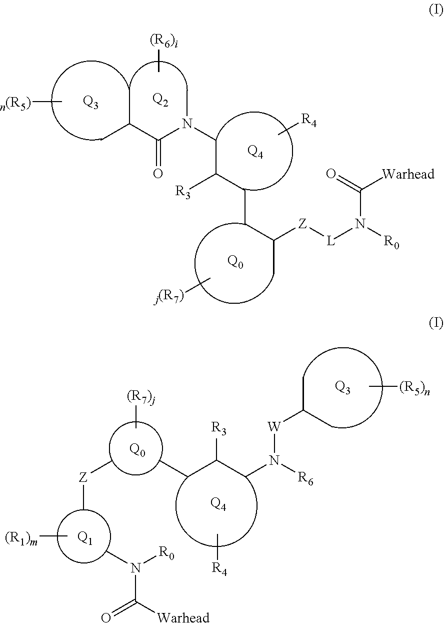

### REFERENCE TO RELATED APPLICATIONS

This application claims priority under 35 USC 119(e) to U.S. Provisional Patent Application Nos. 63/108,094, filed on Oct. 30, 2020, and 63/233,263, filed on Aug. 14, 2021, the entire contents of each of the above applications are incorporated herein by reference.

### BACKGROUND OF THE INVENTION

Bruton tyrosine kinase (Btk) is a Tec family non-receptor protein kinase, expressed in most hematopoietic cells such as B cells, mast cells, and macrophages but not in T cells, natural killer cells, and plasma cells [Smith, C.I. et al. Journal of Immunology (1994), 152 (2), 557-65]. Btk is a crucial part of the BCR and FcR signaling pathway, and the targeted inhibition of Btk is a novel approach for treating many different human diseases such as B-cell malignancies, autoimmune disease, and inflammatory disorders [Uckun, Fatih M. et al, Anti-Cancer Agents in Medicinal Chemistry (2007), Shinohara et al, Cell 132 (2008) pp794-806; Pan, Zhengying, Drug News & Perspectives (2008), 21 (7); 7 (6), 624-632; Gilfillan et al, Immunological Reviews 288 (2009) pp 149-169; Davis et al, Nature, 463 (2010) pp 88-94].

Covalent Bruton's tyrosine kinase (BTK) inhibitors including ibrutinib and acalabrutinib have transformed the treatment landscape of several BTK dependent B-cell malignancies, including chronic lymphocytic leukemia, Waldenstrom's macroglobulinemia, mantle cell lymphoma and marginal zone lymphoma. Despite impressive clinical response of ibrutinib in B-cell malignancies, cases of primary and secondary resistance have emerged with poor outcomes and limited treatment options.

Although BTK inhibitors such as Ibrutinib, and ACP-196, have made a significant contribution to the art, there is a strong need for continuing search in this field of art for highly potent and selective BTK inhibitors.

### SUMMARY OF THE INVENTION

In one aspect, this invention relates to a compound of Formula (I), or an N-oxide thereof, or a pharmaceutically acceptable salt, solvate, polymorph, tautomer, stereoisomer, an isotopic form, or a prodrug of said compound of Formula (I) or N-oxide thereof:

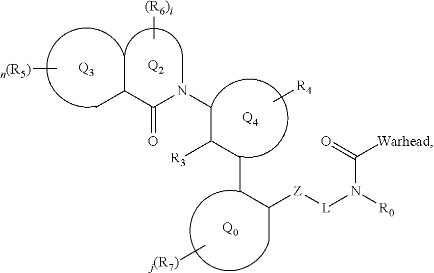

wherein
Q₀ is a 5-9 membered aryl or heteroaryl;
Q₂ is a 5-7 membered heterocycloalkyl;
Q₃ is a 5-membered heteroaryl or phenyl;
Q₄ is a 6-membered heteroaryl;
Z is absent, O, (CH₂)ₚO, O(CH₂)ₚO, N(H), (CH₂)ₚ, S, C(O), S(O₂), OC(O), C(O)O, OSO₂, S(O₂)O, C(O)S, SC(O), C(O)C(O), C(O)N(H), N(H)C(O), S(O₂)N(H), N(H)S(O₂), OC(O)O, OC(O)S, OC(O)N(H), N(H)C(O)O, N(H)C(O)S, N(H)C(O)N(H), (CH₂)ₚN(H)(CH₂)_{q}, (CH₂)ₚO(CH₂)_{q}, (CH₂)ₚN(H)C(O)(CH₂)_{q}, (CH₂)ₚC(O)N(H)(CH₂)_{q}, OC(O)N(H)(CH₂)ₚ₊₁N(H)(CH₂)_{q}, a bivalent alkenyl group, or a bivalent alkynyl group;
L is -L₁-L₂-;
L₁ is a bond, alkyl, alkenyl, alkynyl, cycloalkyl, cycloalkenyl, heterocycloalkyl, heterocycloalkenyl, aryl, or heteroaryl, in which said alkenyl, alkynyl, cycloalkyl, cycloalkenyl, heterocycloalkyl, heterocycloalkenyl, aryl, or heteroaryl is optionally subsitiuted with one or more Rₐ;
L₂ is a bond, or an alkyl in which one or more -Lᵢ- are optionally inserted between any two adjacent carbon atoms;
-Lᵢ- is —N(Rₐ)—, —O—, —S—, —C(O)—, —S(O₂)—, —OC(O)—, —C(O)O—, —OSO₂—, —S(O₂)O—, —C(O)S—, —SC(O)—, —C(O)C(O)—, —C(O)N(Rₐ)—, —N(Rₐ)C(O)—, —S(O₂)N(Rₐ)—, —N(Rₐ)S(O₂)—, —OC(O)O—, —OC(O)S—, —OC(O)N(Rₐ)—, —N(Rₐ)C(O)O—, —N(Rₐ)C(O)S—, —N(Rₐ)C(O)N(Rₐ)—, a bivalent alkenyl group, a bivalent alkynyl group, a bivalent cycloalkyl group, a bivalent heterocycloalkyl group, a bivalent aryl group, a bivalent heteroaryl group;
Warhead is

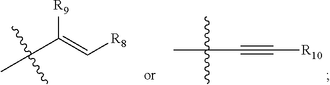

each of R₀, R₅, R₆, R₇, and R₈, independently, is H, D, alkyl, spiroalkyl, alkenyl, alkynyl, cycloalkyl, cycloalkenyl, heterocycloalkyl, spiroheterocycloalkyl, heterocycloalkenyl, aryl, heteroaryl, halo, nitro, oxo, cyano, ORₐ, SRₐ, alkyl-Rₐ, NH(CH₂)ₚRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c}, -alkyl-P(O)R_{b}R_{c}, —S(O)(═N(R_{b}))R_{c}, —N═S(O)R_{b}R_{c}, ═NR_{b}, SO₂N(R_{b})R_{c}, or N(R_{b})SO₂R_{c}, in which said cycloalkyl, cycloalkenyl, heterocycloalkyl, heterocycloalkenyl, aryl, heteroaryl is optionally subsitiuted with one or more R_{d};
R₃ is H, halo, alkyl, haloalkyl, or hydroxyalkyl;
R₄ is H, halo, or low alkyl;
R₉ is H, or cyano;
R₁₀ is H, alkyl, or haloalkyl;
R₀ groups and L, taken together with the atom to which they are attached, may optionally form a cycloalkyl, heterocycloalkyl, aryl, or heteroaryl optionally subsitiuted with one or more R_{d};
two of R₅ groups, taken together with the atom to which they are attached, may optionally form a cycloalkyl, heterocycloalkyl, aryl, or heteroaryl optionally subsitiuted with one or more R_{d};
two of R₆ groups, taken together with the atom to which they are attached, may optionally form a cycloalkyl or heterocycloalkyl optionally subsitiuted with one or more R_{d};
two of R₇ groups, taken together with the atom to which they are attached, may optionally form a cycloalkyl, heterocycloalkyl, aryl, or heteroaryl optionally subsitiuted with one or more R_{d};
Rₐ, R_{b}, R_{c} and R_{d}, independently, is H, D, alkyl, spiroalkyl, alkenyl, alkynyl, halo, cyano, amine, nitro, hydroxy, ═O, —P(O)R_{b}R_{c}, -alkyl-P(O)R_{b}R_{c}, —S(O)(═N(R_{b}))R_{c}, —N═S(O)R_{b}R_{c}, ═NR_{b}, C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl, in which said alkyl, cycloalkyl, cycloalkenyl, heterocycloalkyl, heterocycloalkenyl, aryl, heteroaryl is optionally subsitiuted with one or more Rₑ;
Rₑ is H, D, alkyl, spiroalkyl, alkenyl, alkynyl, halo, cyano, amine, nitro, hydroxy, ═O, C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl;
two of R_{d} groups, taken together with the atom to which they are attached, may optionally form a cycloalkyl or heterocycloalkyl optionally subsitiuted with one or more Rₑ; and
each of i, j, n, p, and q, independently, is 0, 1, 2, 3, or 4.

In a further embodiment, the compound is represented by Formula (II):

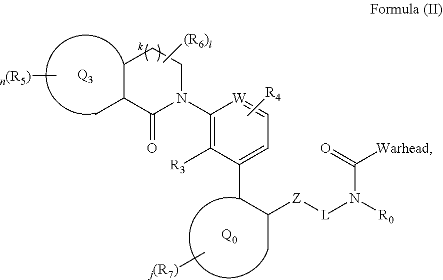

wherein
W is CH or N; and
k is 0, 1, or 2.

In a further embodiment, the compound is represented by Formula (III)

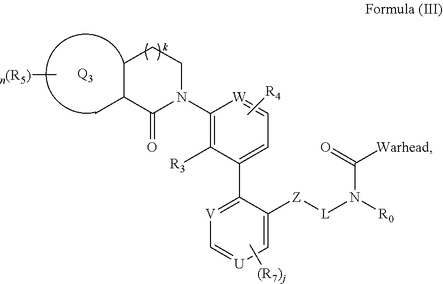

wherein
each of V, U, independently is C(Rₐ) or N.

In a further embodiment, the compound is represented by Formula (IV):

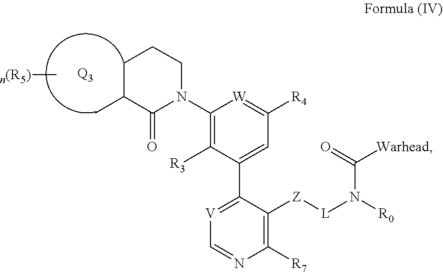

wherein
R₄ is H or F;
Z is O, or

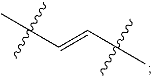 [SMILES: */C=C/*]

and
Warhead is

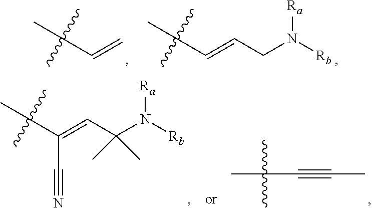

In a further embodiment, the compound is represented by Formula (V):

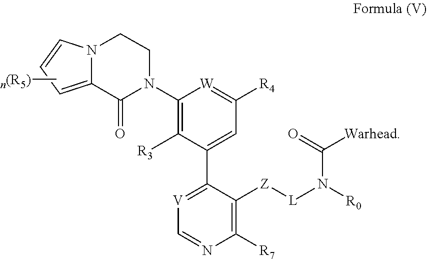

In a further embodiment, the compound is represented by Formula (VI):

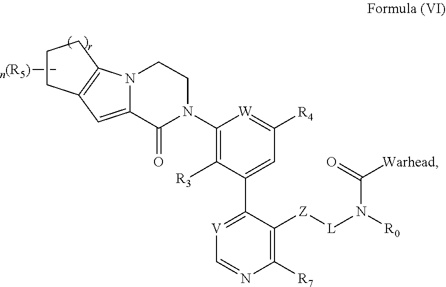

in which each of r, and s, independently, is 0, 1, 2, or 3.

This invention also relates to a compound of Formula (A), or an N-oxide thereof, or a pharmaceutically acceptable salt, solvate, polymorph, tautomer, stereoisomer, an isotopic form, or a prodrug of said compound of Formula (A) or N-oxide thereof

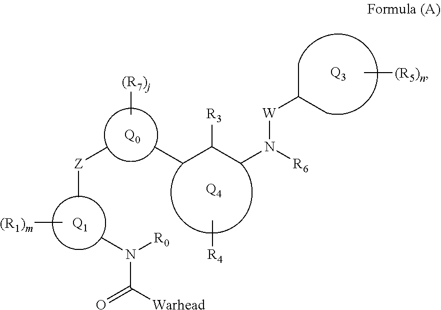

wherein
each of Q₀, Q₃, and Q₄, independently, is a 5-9 membered aryl or 5-9 membered heteroaryl;
Q₄ is a 6-membered heteroaryl;
W is —CH₂—, —C(O)—, or —S(O₂)—;
Z is absent, NH, S, or O;
Warhead is

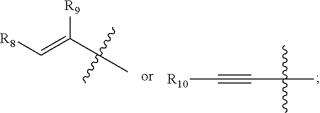

each of R₀, R₁, R₅, R₆, R₇, R₈, R₉, and R₁₀, independently, is H, D, alkyl, spiroalkyl, alkenyl, alkynyl, cycloalkyl, cycloalkenyl, heterocycloalkyl, spiroheterocycloalkyl, heterocycloalkenyl, aryl, heteroaryl, halo, nitro, oxo, cyano, ORₐ, SRₐ, alkyl-Rₐ, NH(CH₂)ₚRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)Rₑ, —P(O)R_{b}R_{c}, -alkyl-P(O)R_{b}R_{c}, —S(O)(═N(R_{b}))R_{c}, —N═S(O)R_{b}R_{c}, ═NR_{b}, SO₂N(R_{b})R_{c}, or N(R_{b})SO₂R_{c}, in which said cycloalkyl, cycloalkenyl, heterocycloalkyl, heterocycloalkenyl, aryl, heteroaryl is optionally subsitiuted with one or more R_{d};
R₃ is H, halo, alkyl, haloalkyl, or hydroxyalkyl;
R₄ is H, halo, or low alkyl;
R₀ and R₁ groups, taken together with the atom to which they are attached, may optionally form a cycloalkyl or heterocycloalkyl optionally subsitiuted with one or more R_{d};
R₃ and R₇ groups, taken together with the atom to which they are attached, may optionally form a cycloalkyl or heterocycloalkyl optionally subsitiuted with one or more R_{d};
two of R₁ groups, taken together with the atom to which they are attached, may optionally form a cycloalkyl or heterocycloalkyl optionally subsitiuted with one or more R_{d};
two of R₅ groups, taken together with the atom to which they are attached, may optionally form a cycloalkyl or heterocycloalkyl optionally subsitiuted with one or more R_{d};
two of R₆ groups, taken together with the atom to which they are attached, may optionally form a cycloalkyl or heterocycloalkyl optionally subsitiuted with one or more R_{d};
two of R₇ groups, taken together with the atom to which they are attached, may optionally form a cycloalkyl or heterocycloalkyl optionally subsitiuted with one or more R_{d};
Rₐ, R_{b}, R_{c} and R_{d}, independently, is H, D, alkyl, spiroalkyl, alkenyl, alkynyl, halo, cyano, amine, nitro, hydroxy, ═O, —P(O)R_{b}R_{c}, -alkyl-P(O)R_{b}R_{c}, —S(O)(═N(R_{b}))Rₑ, —N═S(O)R_{b}R_{c}, ═NR_{b}, C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl, in which said alkyl, cycloalkyl, cycloalkenyl, heterocycloalkyl, heterocycloalkenyl, aryl, heteroaryl is optionally subsitiuted with one or more Rₑ;
Rₑ is H, D, alkyl, spiroalkyl, alkenyl, alkynyl, halo, cyano, amine, nitro, hydroxy, ═O, C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl;
two of R_{d} groups, taken together with the atom to which they are attached, may optionally form a cycloalkyl or heterocycloalkyl optionally subsitiuted with one or more Rₑ; and
each of i, j, m, n, p, and q, independently, is 0, 1, 2, 3, or 4.

In a further embodiment, the compound is represented by Formula (B):

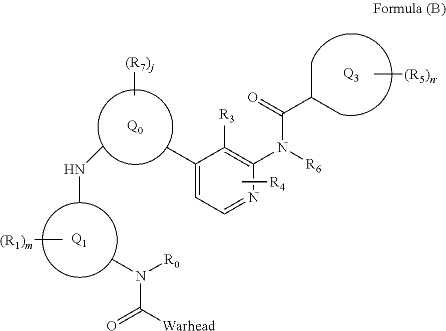

In a further embodiment, the compound is represented by Formula (C)

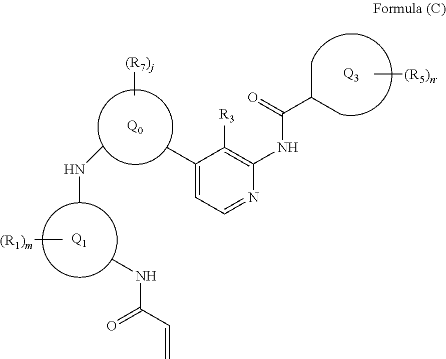

In a further embodiment, the compound is represented by Formula (D):

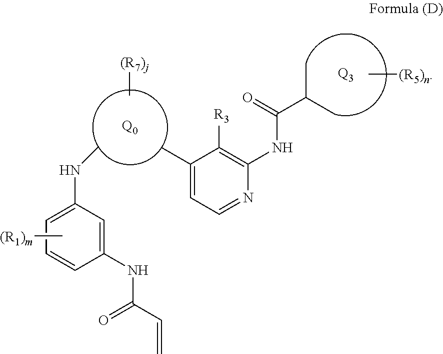

In a further embodiment, the compound is represented by Formula (E):

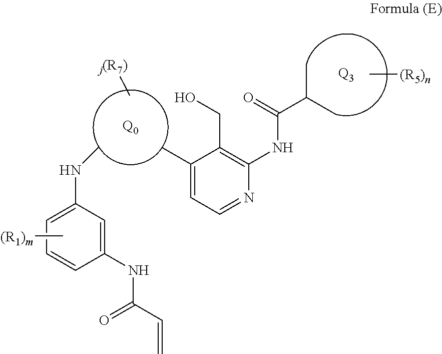

Compounds of the invention may contain one or more asymmetric carbon atoms. Accordingly, the compounds may exist as diastereomers, enantiomers, or mixtures thereof. Each of the asymmetric carbon atoms may be in the R or S configuration, and both of these configurations are within the scope of the invention.

A modified compound of any one of such compounds including a modification having an improved (e.g., enhanced, greater) pharmaceutical solubility, stability, bioavailability, and/or therapeutic index as compared to the unmodified compound is also contemplated. Exemplary modifications include (but are not limited to) applicable prodrug derivatives, and deuterium-enriched compounds.

It should be recognized that the compounds of the present invention may be present and optionally administered in the form of salts or solvates. The invention encompasses any pharmaceutically acceptable salts and solvates of any one of the above-described compounds and modifications thereof.

Also within the scope of this invention is a pharmaceutical composition containing one or more of the compounds, modifications, and/or salts and thereof described above for use in treating a neoplastic disease, autoimmune disease, and inflammatory disorders, therapeutic uses thereof, and use of the compounds for the manufacture of a medicament for treating the disease/disorder.

This invention also relates to a method of treating a neoplastic disease, particularly the B-cell malignancy including but not limited to B-cell lymphoma, lymphoma (including Hodgkin's and non-Hodgkin's lymphoma), hairy cell lymphoma, small lymphocytic lymphoma (SLL), mantle cell lymphoma (MCL), and diffuse large B-cell lymphoma (DLBCL), multiple myeloma, chronic and acute myelogenous leukemia and chronic and acute lymphocytic leukemia, by administering to a subject in need thereof an effective amount of one or more of the compounds, modifications, and/or salts, and compositions thereof described above.

Autoimmune and/or inflammatory diseases that can be affected using compounds and compositions according to the invention include, but are not limited to: psoriasis, allergy, Crohn's disease, irritable bowel syndrome, Sjogren's disease, tissue graft rejection, and hyperacute rejection of transplanted organs, asthma, systemic lupus erythematosus (and associated glomerulonephritis), dermatomyositis, multiple sclerosis, scleroderma, vasculitis (ANCA-associated and other vasculitides), autoimmune hemolytic and thrombocytopenic states, Goodpasture's syndrome (and associated glomerulonephritis and pulmonary hemorrhage), atherosclerosis, rheumatoid arthritis, chronic Idiopathic thrombocytopenic purpura (ITP), Addison's disease, Parkinson's disease, Alzheimer's disease, diabetes, septic shock, and myasthenia gravis.

The details of one or more embodiments of the invention are set forth in the description below. Other features, objects, and advantages of the invention will be apparent from the description and from the claims. It should be understood that all embodiments/features of the invention (compounds, pharmaceutical compositions, methods of make/use, etc) described herein, including any specific features described in the examples and original claims, can combine with one another unless not applicable or explicitly disclaimed.

### DETAILED DESCRIPTION OF THE INVENTION

Exemplary compounds described herein include, but are not limited to, the following:

Part I

(S)-2-(3-(5-((1-acryloylpyrrolidin-2-yl)methoxy)-6-aminopyrimidin-4-yl)-5-fluoro-2-methylphenyl)-7,7-dimethyl-3,4,7,8-tetrahydro-2H-cyclopenta[4,5]pyrrolo[1,2-a]pyrazin-1(6H)-one,

(S,E)-2-(3-(5-(2-(1-acryloylpyrrolidin-2-yl)vinyl)-6-aminopyrimidin-4-yl)-5-fluoro-2-methylphenyl)-7,7-dimethyl-3,4,7,8-tetrahydro-2H-cyclopenta[4,5]pyrrolo[1,2-a]pyrazin-1(6H)-one,

(S)-2-(3-(3-((1-acryloylpyrrolidin-2-yl)methoxy)-2-aminopyridin-4-yl)-5-fluoro-2-methylphenyl)-7,7-dimethyl-3,4,7,8-tetrahydro-2H-cyclopenta[4,5]pyrrolo[1,2-a]pyrazin-1(6H)-one,

(S,E)-2-(3-(3-(2-(1-acryloylpyrrolidin-2-yl)vinyl)-2-aminopyridin-4-yl)-5-fluoro-2-methylphenyl)-7,7-dimethyl-3,4,7,8-tetrahydro-2H-cyclopenta[4,5]pyrrolo[1,2-a]pyrazin-1(6H)-one,

(S)-2-(3′-((1-acryloylpyrrolidin-2-yl)methoxy)-2′-amino-3-methyl-[4,4′-bipyridin]-2-yl)-7,7-dimethyl-3,4,7,8-tetrahydro-2H-cyclopenta[4,5]pyrrolo[1,2-a]pyrazin-1(6H)-one,

(S,E)-2-(3′-(2-(1-acryloylpyrrolidin-2-yl)vinyl)-2′-amino-3-methyl-[4,4′-bipyridin]-2-yl)-7,7-dimethyl-3,4,7,8-tetrahydro-2H-cyclopenta[4,5]pyrrolo[1,2-a]pyrazin-1(6H)-one,

(S)-2-(4-(5-((1-acryloylpyrrolidin-2-yl)methoxy)-6-aminopyrimidin-4-yl)-3-methylpyridin-2-yl)-7,7-dimethyl-3,4,7,8-tetrahydro-2H-cyclopenta[4,5]pyrrolo[1,2-a]pyrazin-1(6H)-one,

(S,E)-2-(4-(5-(2-(1-acryloylpyrrolidin-2-yl)vinyl)-6-aminopyrimidin-4-yl)-3-methylpyridin-2-yl)-7,7-dimethyl-3,4,7,8-tetrahydro-2H-cyclopenta[4,5]pyrrolo[1,2-a]pyrazin-1(6H)-one,

(S)-2-(4-(5-((1-acryloylpyrrolidin-2-yl)methoxy)-6-aminopyrimidin-4-yl)-3-(hydroxymethyl)pyridin-2-yl)-7,7-dimethyl-3,4,7,8-tetrahydro-2H-cyclopenta[4,5]pyrrolo[1,2-a]pyrazin-1(6H)-one,

(S,E)-2-(4-(5-(2-(1-acryloylpyrrolidin-2-yl)vinyl)-6-aminopyrimidin-4-yl)-3-(hydroxymethyl)pyridin-2-yl)-7,7-dimethyl-3,4,7,8-tetrahydro-2H-cyclopenta[4,5]pyrrolo[1,2-a]pyrazin-1(6H)-one,

Part II

(S)-2-(3-(6-amino-5-((1-(but-2-ynoyl)pyrrolidin-2-yl)methoxy)pyrimidin-4-yl)-5-fluoro-2-methylphenyl)-7,7-dimethyl-3,4,7,8-tetrahydro-2H-cyclopenta[4,5]pyrrolo[1,2-a]pyrazin-1(6H)-one,

(S,E)-2-(3-(6-amino-5-((1-(4-(dimethylamino)but-2-enoyl)pyrrolidin-2-yl)methoxy)pyrimidin-4-yl)-5-fluoro-2-methylphenyl)-7,7-dimethyl-3,4,7,8-tetrahydro-2H-cyclopenta[4,5]pyrrolo[1,2-a]pyrazin-1(6H)-one,

(S,E)-2-(3-(6-amino-5-((1-(4-(piperidin-1-yl)but-2-enoyl)pyrrolidin-2-yl)methoxy)pyrimidin-4-yl)-5-fluoro-2-methylphenyl)-7,7-dimethyl-3,4,7,8-tetrahydro-2H-cyclopenta[4,5]pyrrolo[1,2-a]pyrazin-1(6H)-one,

(S,E)-2-(2-(((4-amino-6-(3-(7,7-dimethyl-1-oxo-1,3,4,6,7,8-hexahydro-2H-cyclopenta[4,5]pyrrolo[1,2-a]pyrazin-2-yl)-5-fluoro-2-methylphenyl)pyrimidin-5-yl)methyl)pyrrolidine-1-carbonyl)-4-methyl-4-(4-(oxetan-3-yl)piperazin-1-yl)pent-2-enenitrile,

(S,E)-2-(2-(((4-amino-6-(3-(7,7-dimethyl-1-oxo-1,3,4,6,7,8-hexahydro-2H-cyclopenta[4,5]pyrrolo[1,2-a]pyrazin-2-yl)-5-fluoro-2-methylphenyl)pyrimidin-5-yl)oxy)methyl)pyrrolidine-1-carbonyl)-4-methyl-4-(methyl(oxetan-3-yl)amino)pent-2- enenitrile,

(S)-2-(3-(5-((1-acryloylazetidin-2-yl)methoxy)-6-aminopyrimidin-4-yl)-5-fluoro-2-methylphenyl)-7,7-dimethyl-3,4,7,8-tetrahydro-2H-cyclopenta[4,5]pyrrolo[1,2-a]pyrazin-1(6H)-one,

(S)-2-(3-(5-((1-acryloylpiperidin-2-yl)methoxy)-6-aminopyrimidin-4-yl)-5-fluoro-2-methylphenyl)-7,7-dimethyl-3,4,7,8-tetrahydro-2H-cyclopenta[4,5]pyrrolo[1,2-a]pyrazin-1(6H)-one,

(S)-2-(3-(5-((1-acryloylazepan-2-yl)methoxy)-6-aminopyrimidin-4-yl)-5-fluoro-2-methylphenyl)-7,7-dimethyl-3,4,7,8-tetrahydro-2H-cyclopenta[4,5]pyrrolo[1,2-a]pyrazin-1(6H)-one,

(R)-2-(3-(5-((1-acryloylpyrrolidin-3-yl)methoxy)-6-aminopyrimidin-4-yl)-5-fluoro-2-methylphenyl)-7,7-dimethyl-3,4,7,8-tetrahydro-2H-cyclopenta[4,5]pyrrolo[1,2-a]pyrazin-1(6H)-one,

(R)-2-(3-(5-((1-acryloylpiperidin-3-yl)methoxy)-6-aminopyrimidin-4-yl)-5-fluoro-2-methylphenyl)-7,7-dimethyl-3,4,7,8-tetrahydro-2H-cyclopenta[4,5]pyrrolo[1,2-a]pyrazin-1(6H)-one,

(R)-2-(3-(5-((1-acryloylazepan-3-yl)methoxy)-6-aminopyrimidin-4-yl)-5-fluoro-2-methylphenyl)-7,7-dimethyl-3,4,7,8-tetrahydro-2H-cyclopenta[4,5]pyrrolo[1,2-a]pyrazin-1(6H)-one,

Exemplary compounds described herein include, but are not limited to, the following:

(S)-N-(4-(6-((3-acrylamido-4-(2-methyl-4-(tetrahydro-2H-pyran-4-yl)piperazin-1-yl)phenyl)amino)-4-methyl-5-oxo-4,5-dihydropyrazin-2-yl)-3-(hydroxymethyl)pyridin-2-yl)-4-(tert-butyl)benzamide,

(S)-N-(4-(6-((3-acrylamido-4-(2-methyl-4-(tetrahydro-2H-pyran-4-yl)piperazin-1-yl)phenyl)amino)-4-methyl-5-oxo-4,5-dihydropyrazin-2-yl)-3-(hydroxymethyl)pyridin-2-yl)-4-(tert-butyl)-2-fluorobenzamide,

(S)-N-(4-(6-((3-acrylamido-4-(2-methyl-4-(tetrahydro-2H-pyran-4-yl)piperazin-1-yl)phenyl)amino)-4-methyl-5-oxo-4,5-dihydropyrazin-2-yl)-3-(hydroxymethyl)pyridin-2-yl)-4-cyclopropyl-2-fluorobenzamide,

(S)-N-(4-(6-((3-acrylamido-4-(2-methyl-4-(tetrahydro-2H-pyran-4-yl)piperazin-1-yl)phenyl)amino)-4-methyl-5-oxo-4,5-dihydropyrazin-2-yl)-3-(hydroxymethyl)pyridin-2-yl)-4-cyclopropylbenzamide,

(S)-N-(4-(6-((3-acrylamido-4-(2-methyl-4-(tetrahydro-2H-pyran-4-yl)piperazin-1-yl)phenyl)amino)-4-methyl-5-oxo-4,5-dihydropyrazin-2-yl)-3-(hydroxymethyl)pyridin-2-yl)-1,5,5-trimethyl-1,4,5,6-tetrahydrocyclopenta[b]pyrrole-2-carboxamide,

(S)-N-(4-(6-((3-acrylamido-4-(2-methyl-4-(tetrahydro-2H-pyran-4-yl)piperazin-1-yl)phenyl)amino)-4-methyl-5-oxo-4,5-dihydropyrazin-2-yl)-3-(hydroxymethyl)pyridin-2-yl)-5,5-dimethyl-1,4,5,6-tetrahydrocyclopenta[b]pyrrole-2-carboxamide,

(S)-N-(4-(6-((3-acrylamido-4-(2-methyl-4-(tetrahydro-2H-pyran-4-yl)piperazin-1-yl)phenyl)amino)-4-methyl-5-oxo-4,5-dihydropyrazin-2-yl)-3-(hydroxymethyl)pyridin-2-yl)-5-(tert-butyl)thiophene-2-carboxamide,

(S)-N-(4-(6-((3-acrylamido-4-(2-methyl-4-(tetrahydro-2H-pyran-4-yl)piperazin-1-yl)phenyl)amino)-4-methyl-5-oxo-4,5-dihydropyrazin-2-yl)-3-(hydroxymethyl)pyridin-2-yl)-3-methyl-4,5,6,7-tetrahydrobenzo[b]thiophene-2-carboxamide,

(S)-N-(4-(6-((3-acrylamido-4-(2-methyl-4-(tetrahydro-2H-pyran-4-yl)piperazin-1-yl)phenyl)amino)-4-methyl-5-oxo-4,5-dihydropyrazin-2-yl)-3-(hydroxymethyl)pyridin-2-yl)-4,5,6,7-tetrahydrobenzo[b]thiophene-2-carboxamide,

(S)-N-(6-((3-acrylamido-4-(2-methyl-4-(tetrahydro-2H-pyran-4-yl)piperazin-1-yl)phenyl)amino)-3′-(hydroxymethyl)-[2,4′-hipyridin]-2′-yl)-4-(tert-butyl)-2-fluorobenzamide,

(S)-N-(4-(6-((3-acrylamido-4-(2-methyl-4-(tetrahydro-2H-pyran-4-yl)piperazin-1-yl)phenyl)amino)pyrazin-2-yl)-3-(hydroxymethyl)pyridin-2-yl)-4-(tert-butyl)-2-fluorobenzamide,

(S)-N-(4-(2-((3-acrylamido-4-(2-methyl-4-(tetrahydro-2H-pyran-4-yl)piperazin-1-yl)phenyl)amino)pyrimidin-4-yl)-3-(hydroxymethyl)pyridin-2-yl)-4-cyclopropyl-2-fluorobenzamide,

(S)-N-(4-(2-((3-acrylamido-4-(2-methyl-4-(tetrahydro-2H-pyran-4-yl)piperazin-1-yl)phenyl)amino)-1,3,5-triazin-2-yl)-3-(hydroxymethyl)pyridin-2-yl)-4-cyclopropyl-2-fluorobenzamide,

(S)-N-(4-(4-((3-acrylamido-4-(2-methyl-4-(tetrahydro-2H-pyran-4-yl)piperazin-1-yl)phenyl)amino)pyrimidin-2-yl)-3-(hydroxymethyl)pyridin-2-yl)-4-(tert-butyl)-2-fluorobenzamide,

(S)-N-(4-(4-((3-acrylamido-4-(2-methyl-4-(tetrahydro-2H-pyran-4-yl)piperazin-1-yl)phenyl)amino)-5-(trifluoromethyl)pyrimidin-2-yl)-3-(hydroxymethyl)pyridin-2-yl)-4-(tert-butyl)-2-fluorobenzamide,

(S)-N-(4-(4-((3-acrylamido-4-(2-methyl-4-(tetrahydro-2H-pyran-4-yl)piperazin-1-yl)phenyl)amino)-5-methoxypyrimidin-2-yl)-3-(hydroxymethyl)pyridin-2-yl)-4-(tert-butyl)-2-fluorobenzamide,

(S)-N-(4-(4-( (3-acrylamido-4-(2-methyl-4-(tetrahydro-2H-pyran-4-yl)piperazin-1-yl)phenyl)amino)-5-isopropoxypyrimidin-2-yl)-3-(hydroxymethyl)pyridin-2-yl)-4-(tert-butyl)-2-fluorobenzamide,

(S)-N-(4-(4-((3-acrylamido-4-(2-methyl-4-(tetrahydro-2H-pyran-4-yl)piperazin-1-yl)phenyl)amino)-5-aminopyrimidin-2-yl)-3-(hydroxymethyl)pyridin-2-yl)-4-(tert-butyl)-2-fluorobenzamide,

(S)-N-(4-(4-)((3-acrylamido-4-(2-methyl-4-(tetrahydro-2H-pyran-4-yl)piperazin-1-yl)phenyl)amino)-5-(methylamino)pyrimidin-2-yl)-3-(hydroxymethyl)pyridin-2-yl)-4-(tert-butyl)-2-fluorobenzamide,

(S)-3-((3 -acrylamido-4-(2-methyl-4-(tetrahydro-2H-pyran-4-yl)piperazin-1-yl)phenyl)amino)-1-(2-(4-cyclopropyl-2-fluorobenzamido)-3-(hydroxymethyl)pyridin-4-yl)-1H-pyrazole-4-carboxamide,

(S)-N-(4-(3-((3-acrylamido-4-(2-methyl-4-(tetrahydro-2H-pyran-4-yl)piperazin-1-yl)phenyl)amino)-4-oxo-4,5,6,7-tetrahydro-1H-pyrazolo [4,3 -c]pyridin-1-yl)-3-(hydroxymethyl)pyridin-2-yl)-4-cyclopropyl-2-fluorobenzamide,

(S)-N-(4-(8-((3-acrylamido-4-(2-methyl-4-(tetrahydro-2H-pyran-4-yl)piperazin-1-yl)phenyl)amino)imidazo[1,2-a]pyrazin-6-yl)-3-(hydroxymethyl)pyridin-2-yl)-4-(tert-butyl)-2-fluorobenzamide,

(S)-N-(4-(6-((3-acrylamido-4-(2-methyl-4-(oxetan-3-yl)piperazin-1-yl)phenyl)amino)-4-methyl-5-oxo-4,5-dihydropyrazin-2-yl)-3-(hydroxymethyl)pyridin-2-yl)-4-cyclopropyl-2-fluorobenzamide,

(R)-N-(4-(6-((3-acrylamido-4-(4-(oxetan-3 -yl)-2-(trifluoromethyl)piperazin-1-yl)phenyl)amino)-4-methyl-5-oxo-4,5-dihydropyrazin-2-yl)-3-(hydroxymethyl)pyridin-2-yl)-4-cyclopropyl-2-fluorobenzamide,

(S)-N-(4-(6-((3-acrylamido-4-(2-methyl-4-(tetrahydro-2H-pyran-4-yl)piperazin-1-yl)phenyl)amino)-4-methyl-5-oxo-4,5-dihydropyrazin-2-yl)-3-methylpyridin-2-yl)-4-cyclopropyl-2-fluorobenzamide,

(S)-N-(4-(6-((5-acrylamido-6-(2-methyl-4-(tetrahydro-2H-pyran-4-yl)piperazin-1-yl)pyridin-3-yl)amino)-4-methyl-5-oxo-4,5-dihydropyrazin-2-yl)-3-(hydroxymethyl)pyridin-2-yl)-4-cyclopropyl-2-fluorobenzamide,

(S)-N-(4-(6-((4-acrylamido-5-(2-methyl-4-(tetrahydro-2H-pyran-4-yl)piperazin-1-yl)thiophen-2-yl)amino)-4-methyl-5-oxo-4,5-dihydropyrazin-2-yl)-3-(hydroxymethyl)pyridin-2-yl)-4-cyclopropyl-2-fluorobenzamide,

(S)-4-cyclopropyl-2-fluoro-N-(3 -(hydroxymethyl)-4-(4-methyl-6-((4-(2-methyl-4-(tetrahydro-2H-pyran-4-yl)piperazin-1-yl)-3-(N-methylacrylamido)phenyl)amino)-5-oxo-4,5-dihydropyrazin-2-yl)pyridin-2-yl)benzamide,

(S)-N-(4-(6-((3-(but-2-ynamido)-4-(2-methyl-4-(tetrahydro-2H-pyran-4-yl)piperazin-1-yl)phenyl)amino)-4-methyl-5-oxo-4,5-dihydropyrazin-2-yl)-3-(hydroxymethyl)pyridin-2-yl)-4-cyclopropyl-2-fluorobenzamide,

(S)-4-cyclopropyl-2-fluoro-N-(3 -(hydroxymethyl)-4-(4-methyl-6-((4-(2-methyl-4-(tetrahydro-2H-pyran-4-yl)piperazin-1-yl)-3-propiolamidophenyl)amino)-5-oxo-4,5-dihydropyrazin-2-yl)pyridin-2-yl)benzamide,

(S,E)-4-cyclopropyl-N-(4-(6-((3-(4-(dimethylamino)but-2-enamido)-4-(2-methyl-4-(tetrahydro-2H-pyran-4-yl)piperazin-1-yl)phenyl)amino)-4-methyl-5-oxo-4,5-dihydropyrazin-2-yl)-3-(hydroxymethyl)pyridin-2-yl)-2-fluorobenzamide,

(S,E)-N-(4-(6-((3-(2-cyano-4-methyl-4-(4-methylpiperazin-1-yl)pent-2-enamido)-4-(2-methyl-4-(tetrahydro-2H-pyran-4-yl)piperazin-1-yl)phenyl)amino)-4-methyl-5-oxo-4,5-dihydropyrazin-2-yl)-3-(hydroxymethyl)pyridin-2-yl)-4-cyclopropyl-2-fluorobenzamide,

(S)-N-(4-(6-((3-acrylamido-4-(2-methyl-4-(tetrahydro-2H-pyran-4-yl)piperazin-1-yl )phenyl)amino)-4-methyl-5-oxo-4,5-dihydropyrazin-2-yl)-3-(hydroxymethyl)pyridin-2-yl)-4-cyclopropyl-2-fluoro-N-methylbenzamide,

(S)-N-(4-(6-( (3-acrylamido-4-(2-methyl-4-(tetrahydro-2H-pyran-4-yl)piperazin-1-yl)phenyl)amino)-4-methyl-5-oxo-4,5-dihydropyrazin-2-yl)-3-(hydroxymethyl)pyridin-2-yl)-4-cyclopropyl-N-ethyl-2-fluorobenzamide,

(S)-N-(4-(6-(3-acrylamido-4-(2-methyl-4-(tetrahydro-2H-pyran-4-yl)piperazin-1-yl)phenoxy)-4-methyl-5-oxo-4,5-dihydropyrazin-2-yl)-3-(hydroxymethyl)pyridin-2-yl)-4-cyclopropyl-2-fluorobenzamide,

(S)-N-(4-(6-(3-acrylamido-4-(2-methyl-4-(tetrahydro-2H-pyran-4-yl)piperazin-1-yl)phenoxy)-4-methyl-5-oxo-4,5-dihydropyrazin-2-yl)-3-(hydroxymethyl)pyridin-2-yl)-4-cyclopropyl-2-fluorobenzamide,

(S)-N-(4-(6-(3-acrylamido-4-(2-methyl-4-(oxetan-3-yl)piperazin-1-yl)phenyl)-7H-pyrrolo[2,3-d]pyrimidin-4-yl)-3-(hydroxymethyl)pyridin-2-yl)-4-cyclopropyl-2-fluorobenzamide,

(S)-N-(3-(6-((3-acrylamido-4-(2-methyl-4-(tetrahydro-2H-pyran-4-yl)piperazin-1-yl)phenyl)amino)-4-methyl-5-oxo-4,5-dihydropyrazin-2-yl)-2-(hydroxymethyl)phenyl)-4-cyclopropyl-2-fluorobenzamide,

(S)-N-(3-(6-((3-acrylamido-4-(2-methyl-4-(tetrahydro-2H-pyran-4-yl)piperazin-1-yl)phenyl)amino)-4-methyl-5-oxo-4,5-dihydropyrazin-2-yl)-5-fluoro-2-(hydroxymethyl)phenyl)-4-cyclopropyl-2-fluorobenzamide,

(S)-N-(3-(6-((3-acrylamido-4-(2-methyl-4-(tetrahydro-2H-pyran-4-yl)piperazin-1-yl)phenyl)amino)-4-methyl-5 -oxo-4,5-dihydropyrazin-2-yl)-2-methylphenyl)-4-cyclopropyl-2-fluorobenzamide,

(S)-N-(5-((6-(2-((4-cyclopropyl-2-fluorophenyl)sulfonamido)-3-(hydroxymethyl)pyridin-4-yl)-4-methyl-3-oxo-3,4-dihydropyrazin-2-yl)amino)-2-(2-methyl-4-(tetrahydro-2H-pyran-4-yl)piperazin-1-yl)phenyl)acrylamide,

(S)-N-(5-(((6-(2-((4-cyclopropyl-2-fluorobenzyl)amino)-3-(hydroxymethyl)pyridin-4-yl)-4-methyl-3-oxo-3,4-dihydropyrazin-2-yl)amino)-2-(2-methyl-4-(tetrahydro-2H-pyran-4-yl)piperazin-1-yl)phenyl)acrylamide.

Compounds of the invention may contain one or more asymmetric carbon atoms. Accordingly, the compounds may exist as diastereomers, enantiomers or mixtures thereof. The syntheses of the compounds may employ racemates, diastereomers or enantiomers as starting materials or as intermediates. Diastereomeric compounds may be separated by chromatographic or crystallization methods. Similarly, enantiomeric mixtures may be separated using the same techniques or others known in the art. Each of the asymmetric carbon atoms may be in the R or S configuration and both of these configurations are within the scope of the invention.

A modified compound of any one of such compounds including a modification having an improved (e.g., enhanced, greater) pharmaceutical solubility, stability, bioavailability and/or therapeutic index as compared to the unmodified compound is also contemplated. The examples of modifications include but not limited to the prodrug derivatives, and the deuterium-enriched compounds. For example:
Prodrug derivatives: prodrugs, upon administration to a subject, will converted in vivo into active compounds of the present invention [Nature Reviews of Drug Discovery, 2008, Volume 7, p255]. It is noted that in many instances, the prodrugs themselves also fall within the scope of the range of compounds according to the present invention. The prodrugs of the compounds of the present invention can be prepared by standard organic reaction, for example, by reacting with a carbamylating agent (e.g., 1,1-acyloxyalkylcarbonochloridate, para-nitrophenyl carbonate, or the like) or an acylating agent. Further examples of methods and strategies of making prodrugs are described in Bioorganic and Medicinal Chemistry Letters, 1994, Vol. 4, p. 1985.
Deuterium-enriched compounds: deuterium (D or ² H) is a stable, non-radioactive isotope of hydrogen and has an atomic weight of 2.0144. Hydrogen naturally occurs as a mixture of the isotopes ^{X}H (hydrogen or protium), D (²H or deuterium), and T (³H or tritium). The natural abundance of deuterium is 0.015%. One of ordinary skill in the art recognizes that in all chemical compounds with a H atom, the H atom actually represents a mixture of H and D, with about 0.015% being D. Thus, compounds with a level of deuterium that has been enriched to be greater than its natural abundance of 0.015%, should be considered unnatural and, as a result, novel over their nonenriched counterparts.

It should be recognized that the compounds of the present invention may be present and optionally administered in the form of salts, and solvates. For example, it is within the scope of the present invention to convert the compounds of the present invention into and use them in the form of their pharmaceutically acceptable salts derived from various organic and inorganic acids and bases in accordance with procedures well known in the art.

When the compounds of the present invention possess a free base form, the compounds can be prepared as a pharmaceutically acceptable acid addition salt by reacting the free base form of the compound with a pharmaceutically acceptable inorganic or organic acid, e.g., hydrohalides such as hydrochloride, hydrobromide, hydroiodide; other mineral acids such as sulfate, nitrate, phosphate, etc.; and alkyl and monoarylsulfonates such as ethanesulfonate, toluenesulfonate and benzenesulfonate; and other organic acids and their corresponding salts such as acetate, tartrate, maleate, succinate, citrate, benzoate, salicylate and ascorbate. Further acid addition salts of the present invention include, but are not limited to: adipate, alginate, arginate, aspartate, bisulfate, bisulfite, bromide, butyrate, camphorate, camphorsulfonate, caprylate, chloride, chlorobenzoate, cyclopentanepropionate, digluconate, dihydrogenphosphate, dinitrobenzoate, dodecylsulfate, fumarate, galacterate (from mucic acid), galacturonate, glucoheptaoate, gluconate, glutamate, glycerophosphate, hemisuccinate, hemisulfate, heptanoate, hexanoate, hippurate, 2-hydroxyethanesulfonate, iodide, isethionate, iso-butyrate, lactate, lactobionate, malonate, mandelate, metaphosphate, methanesulfonate, methylbenzoate, monohydrogenphosphate, 2-naphthalenesulfonate, nicotinate, oxalate, oleate, pamoate, pectinate, persulfate, phenylacetate, 3-phenylpropionate, phosphonate and phthalate. It should be recognized that the free base forms will typically differ from their respective salt forms somewhat in physical properties such as solubility in polar solvents, but otherwise the salts are equivalent to their respective free base forms for the purposes of the present invention.

When the compounds of the present invention possess a free acid form, a pharmaceutically acceptable base addition salt can be prepared by reacting the free acid form of the compound with a pharmaceutically acceptable inorganic or organic base. Examples of such bases are alkali metal hydroxides including potassium, sodium and lithium hydroxides; alkaline earth metal hydroxides such as barium and calcium hydroxides; alkali metal alkoxides, e.g., potassium ethanolate and sodium propanolate; and various organic bases such as ammonium hydroxide, piperidine, diethanolamine and N-methylglutamine. Also included are the aluminum salts of the compounds of the present invention. Further base salts of the present invention include, but are not limited to: copper, ferric, ferrous, lithium, magnesium, manganic, manganous, potassium, sodium and zinc salts. Organic base salts include, but are not limited to, salts of primary, secondary and tertiary amines, substituted amines including naturally occurring substituted amines, cyclic amines and basic ion exchange resins, e.g., arginine, betaine, caffeine, chloroprocaine, choline, N,N′-dibenzylethylenediamine (benzathine), dicyclohexylamine, diethanolamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris-(hydroxymethyl)-methylamine (tromethamine). It should be recognized that the free acid forms will typically differ from their respective salt forms somewhat in physical properties such as solubility in polar solvents, but otherwise the salts are equivalent to their respective free acid forms for the purposes of the present invention.

In one aspect, a pharmaceutically acceptable salt is a hydrochloride salt, hydrobromide salt, methanesulfonate, toluenesulfonate, acetate, fumarate, sulfate, bisulfate, succinate, citrate, phosphate, maleate, nitrate, tartrate, benzoate, biocarbonate, carbonate, sodium hydroxide salt, calcium hydroxide salt, potassium hydroxide salt, tromethamine salt, or mixtures thereof.

Compounds of the present invention that comprise tertiary nitrogen-containing groups may be quaternized with such agents as (C₁₋₄) alkyl halides, e.g., methyl, ethyl, iso-propyl and tert-butyl chlorides, bromides and iodides; di-(C₁₋₄) alkyl sulfates, e.g., dimethyl, diethyl and diamyl sulfates; alkyl halides, e.g., decyl, dodecyl, lauryl, myristyl and stearyl chlorides, bromides and iodides; and aryl (C₁₋₄) alkyl halides, e.g., benzyl chloride and phenethyl bromide. Such salts permit the preparation of both water- and oil-soluble compounds of the invention.

Amine oxides, also known as amine-N-oxide and N-oxide, of anti-cancer agents with tertiary nitrogen atoms have been developed as prodrugs [Mol Cancer Therapy. 2004 Mar. 3(3):233-44]. Compounds of the present invention that comprise tertiary nitrogen atoms may be oxidized by such agents as hydrogen peroxide (H₂O₂), Caro's acid or peracids like meta-Chloroperoxybenzoic acid (mCPBA) to from amine oxide.

The invention encompasses pharmaceutical compositions comprising the compound of the present invention and pharmaceutical excipients, as well as other conventional pharmaceutically inactive agents. Any inert excipient that is commonly used as a carrier or diluent may be used in compositions of the present invention, such as sugars, polyalcohols, soluble polymers, salts and lipids. Sugars and polyalcohols which may be employed include, without limitation, lactose, sucrose, mannitol, and sorbitol. Illustrative of the soluble polymers which may be employed are polyoxyethylene, poloxamers, polyvinylpyrrolidone, and dextran. Useful salts include, without limitation, sodium chloride, magnesium chloride, and calcium chloride. Lipids which may be employed include, without limitation, fatty acids, glycerol fatty acid esters, glycolipids, and phospholipids.

In addition, the pharmaceutical compositions may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCL, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol, cyclodextrins), a glidant (e.g., colloidal silicon dioxide), anti-oxidants (e.g., ascorbic acid, sodium metabisulfite, butylated hydroxyanisole), stabilizers (e.g., hydroxypropyl cellulose, hydroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate, methyl cellulose, hydroxyethyl cellulose, carboxymethylcellulose sodium), polymer coatings (e.g., poloxamers or poloxamines), coating and film forming agents (e.g., ethyl cellulose, acrylates, polymethacrylates) and/or adjuvants.

In one embodiment, the pharmaceutical compositions are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Additionally, the invention encompasses pharmaceutical compositions comprising any solid or liquid physical form of the compound of the invention. For example, the compounds can be in a crystalline form, in amorphous form, and have any particle size. The particles may be micronized, or may be agglomerated, particulate granules, powders, oils, oily suspensions or any other form of solid or liquid physical form.

When compounds according to the present invention exhibit insufficient solubility, methods for solubilizing the compounds may be used. Such methods are known to those of skill in this art, and include, but are not limited to, pH adjustment and salt formation, using co-solvents, such as ethanol, propylene glycol, polyethylene glycol (PEG) 300, PEG 400, DMA (10-30%), DMSO (10-20%), NMP (10-20%), using surfactants, such as polysorbate 80, polysorbate 20 (1-10%), cremophor EL, Cremophor RH40, Cremophor RH60 (5-10%), Pluronic F68/Poloxamer 188 (20-50%), Solutol HS15 (20-50%), Vitamin E TPGS, and d-α-tocopheryl PEG 1000 succinate (20-50%), using complexation such as HPfβCD and SBEfβCD (10-40%), and using advanced approaches such as micelle, addition of a polymer, nanoparticle suspensions, and liposome formation.

A wide variety of administration methods may be used in conjunction with the compounds of the present invention. Compounds of the present invention may be administered or coadministered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds according to the invention may also be administered or coadministered in slow release dosage forms. Compounds may be in gaseous, liquid, semi-liquid or solid form, formulated in a manner suitable for the route of administration to be used. For oral administration, suitable solid oral formulations include tablets, capsules, pills, granules, pellets, sachets and effervescent, powders, and the like. Suitable liquid oral formulations include solutions, suspensions, dispersions, emulsions, oils and the like. For parenteral administration, reconstitution of a lyophilized powder is typically used.

As used herein, “Acyl” means a carbonyl containing substituent represented by the formula —C(O)—R in which R is H, alkyl, a carbocycle, a heterocycle, carbocycle-substituted alkyl or heterocycle-substituted alkyl wherein the alkyl, alkoxy, carbocycle and heterocycle are as defined herein. Acyl groups include alkanoyl (e.g. acetyl), aroyl (e.g. benzoyl), and heteroaroyl.

“Aliphatic” means a moiety characterized by a straight or branched chain arrangement of constituent carbon atoms and may be saturated or partially unsaturated with one or more double or triple bonds.

The term “alkyl” refers to a straight or branched hydrocarbon containing 1-20 carbon atoms (e.g., C₁-C₁₀). Examples of alkyl include, but are not limited to, methyl, methylene, ethyl, ethylene, n-propyl, i-propyl, n-butyl, i-butyl, and t-butyl. Preferably, the alkyl group has one to ten carbon atoms. More preferably, the alkyl group has one to four carbon atoms.

The term “alkenyl” refers to a straight or branched hydrocarbon containing 2-20 carbon atoms (e.g., C₂-C₁₀) and one or more double bonds. Examples of alkenyl include, but are not limited to, ethenyl, propenyl, and allyl. Preferably, the alkylene group has two to ten carbon atoms. More preferably, the alkylene group has two to four carbon atoms.

The term “alkynyl” refers to a straight or branched hydrocarbon containing 2-20 carbon atoms (e.g., C₂-C₁₀) and one or more triple bonds. Examples of alkynyl include, but are not limited to, ethynyl, 1-propynyl, 1- and 2-butynyl, and 1-methyl-2-butynyl. Preferably, the alkynyl group has two to ten carbon atoms. More preferably, the alkynyl group has two to four carbon atoms.

The term “alkylamino” refers to an —N(R)-alkyl in which R can be H, alkyl, alkenyl, alkynyl, cycloalkyl, cycloalkenyl, heterocycloalkyl, heterocycloalkenyl, aryl, or heteroaryl.

“Alkoxy” means an oxygen moiety having a further alkyl substituent.

“Alkoxycarbonyl” means an alkoxy group attached to a carbonyl group.

“Oxoalkyl” means an alkyl, further substituted with a carbonyl group. The carbonyl group may be an aldehyde, ketone, ester, amide, acid or acid chloride.

The term “cycloalkyl” refers to a saturated hydrocarbon ring system having 3 to 30 carbon atoms (e.g., C₃-C₁₂,C₃-C₈, C₃-C₆). Examples of cycloalkyl include, but are not limited to, cyclopropyl, cyclobutyl, cyclopentyl, cyclohexyl, cycloheptyl, and cyclooctyl. The term “cycloalkenyl” refers to a non-aromatic hydrocarbon ring system having 3 to 30 carbons (e.g., C₃-C₁₂) and one or more double bonds. Examples include cyclopentenyl, cyclohexenyl, and cycloheptenyl.

The term “heterocycloalkyl” refers to a nonaromatic 5-8 membered monocyclic, 8-12 membered bicyclic, or 11-14 membered tricyclic ring system having one or more heteroatoms (such as O, N, S, P, or Se). Examples of heterocycloalkyl groups include, but are not limited to, piperazinyl, pyrrolidinyl, dioxanyl, morpholinyl, and tetrahydrofuranyl.

The term “heterocycloalkenyl” refers to a nonaromatic 5-8 membered monocyclic, 8-12 membered bicyclic, or 11-14 membered tricyclic ring system having one or more heteroatoms (such as O, N, S, P, or Se) and one or more double bonds.

The term “aryl” refers to a 6-carbon monocyclic, 10-carbon bicyclic, 14-carbon tricyclic aromatic ring system. Examples of aryl groups include, but are not limited to, phenyl, naphthyl, and anthracenyl. The term “heteroaryl” refers to an aromatic 5-8 membered monocyclic, 8-12 membered bicyclic, or 11-14 membered tricyclic ring system having one or more heteroatoms (such as O, N, S, P, or Se). Examples of heteroaryl groups include pyridyl, furyl, imidazolyl, benzimidazolyl, pyrimidinyl, thienyl, quinolinyl, indolyl, and thiazolyl.

Alkyl, alkenyl, alkynyl, cycloalkyl, heterocycloalkyl, cycloalkenyl, heterocycloalkenyl, alkylamino, aryl, and heteroaryl mentioned above include both substituted and unsubstituted moieties. Possible substituents on alkylamino, cycloalkyl, heterocycloalkyl, cycloalkenyl, heterocycloalkenyl, aryl, and heteroaryl include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl, aryloxy, heteroaryl, heteroaryloxy, amino, C₁-C₁₀ alkylamino, arylamino, hydroxy, halo, oxo (O═), thioxo (S═), thio, silyl, C₁-C₁₀ alkylthio, arylthio, C₁-C₁₀ alkylsulfonyl, arylsulfonyl, acylamino, aminoacyl, aminothioacyl, amidino, mercapto, amido, thioureido, thiocyanato, sulfonamido, guanidine, ureido, cyano, nitro, acyl, thioacyl, acyloxy, carbamido, carbamyl, carboxyl, and carboxylic ester. On the other hand, possible substituents on alkyl, alkenyl, or alkynyl include all of the above-recited substituents except C₁-C₁₀ alkyl. Cycloalkyl, cycloalkenyl, heterocycloalkyl, heterocycloalkenyl, aryl, and heteroaryl can also be fused with each other.

“Amino” means a nitrogen moiety having two further substituents where each substituent has a hydrogen or carbon atom alpha bonded to the nitrogen. Unless indicated otherwise, the compounds of the invention containing amino moieties may include protected derivatives thereof. Suitable protecting groups for amino moieties include acetyl, tert-butoxycarbonyl, benzyloxycarbonyl, and the like.

“Aromatic” means a moiety wherein the constituent atoms make up an unsaturated ring system, all atoms in the ring system are sp2 hybridized and the total number of pi electrons is equal to 4n+2. An aromatic ring may be such that the ring atoms are only carbon atoms or may include carbon and non-carbon atoms (see Heteroaryl).

“Carbamoyl” means the radical —OC(O)NRₐR_{b} where Rₐ and R_{b} are each independently two further substituents where a hydrogen or carbon atom is alpha to the nitrogen. It is noted that carbamoyl moieties may include protected derivatives thereof. Examples of suitable protecting groups for carbamoyl moieties include acetyl, tert-butoxycarbonyl, benzyloxycarbonyl, and the like. It is noted that both the unprotected and protected derivatives fall within the scope of the invention.

“Carbonyl” means the radical —C(O)—. It is noted that the carbonyl radical may be further substituted with a variety of substituents to form different carbonyl groups including acids, acid halides, amides, esters, and ketones.

“Carboxy” means the radical —C(O)O—. It is noted that compounds of the invention containing carboxy moieties may include protected derivatives thereof, i.e., where the oxygen is substituted with a protecting group. Suitable protecting groups for carboxy moieties include benzyl, tert-butyl, and the like.

“Cyano” means the radical —CN.

“Formyl” means the radical —CH═O.

“Formimino” means the radical —HC═NH.

“Halo” means fluoro, chloro, bromo or iodo.

“Halo-substituted alkyl”, as an isolated group or part of a larger group, means “alkyl” substituted by one or more “halo” atoms, as such terms are defined in this Application. Halo-substituted alkyl includes haloalkyl, dihaloalkyl, trihaloalkyl, perhaloalkyl and the like.

“Hydroxy” means the radical —OH.

“Imine derivative” means a derivative comprising the moiety —C(═NR)—, wherein R comprises a hydrogen or carbon atom alpha to the nitrogen.

“Isomers” mean any compound having identical molecular formulae but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are nonsuperimposable mirror images are termed “enantiomers” or sometimes “optical isomers.” A carbon atom bonded to four nonidentical substituents is termed a “chiral center.” A compound with one chiral center has two enantiomeric forms of opposite chirality. A mixture of the two enantiomeric forms is termed a “racemic mixture.”

“Nitro” means the radical —NO₂.

“Protected derivatives” means derivatives of compounds in which a reactive site are blocked with protecting groups. Protected derivatives are useful in the preparation of pharmaceuticals or in themselves may be active as inhibitors. A comprehensive list of suitable protecting groups can be found in T. W. Greene, Protecting Groups in Organic Synthesis, 3rd edition, Wiley & Sons, 1999.

The term “substituted” means that an atom or group of atoms has replaced hydrogen as the substituent attached to another group. For aryl and heteroaryl groups, the term “substituted” refers to any level of substitution, namely mono-, di-, tri-, tetra-, or penta-substitution, where such substitution is permitted. The substituents are independently selected, and substitution may be at any chemically accessible position. The term “unsubstituted” means that a given moiety may consist of only hydrogen substituents through available valencies (unsubstituted).

If a functional group is described as being “optionally substituted,” the function group may be either (1) not substituted, or (2) substituted. If a carbon of a functional group is described as being optionally substituted with one or more of a list of substituents, one or more of the hydrogen atoms on the carbon (to the extent there are any) may separately and/or together be replaced with an independently selected optional substituent.

“Sulfide” means —S—R wherein R is H, alkyl, carbocycle, heterocycle, carbocycloalkyl or heterocycloalkyl. Particular sulfide groups are mercapto, alkylsulfide, for example methylsulfide (—S—Me); arylsulfide, e.g., phenylsulfide; aralkylsulfide, e.g., benzylsulfide.

“Sulfinyl” means the radical —S(O)—. It is noted that the sulfinyl radical may be further substituted with a variety of substituents to form different sulfinyl groups including sulfinic acids, sulfinamides, sulfinyl esters, and sulfoxides.

“Sulfonyl” means the radical —S(O)(O)—. It is noted that the sulfonyl radical may be further substituted with a variety of substituents to form different sulfonyl groups including sulfonic acids, sulfonamides, sulfonate esters, and sulfones.

“Thiocarbonyl” means the radical —C(S)—. It is noted that the thiocarbonyl radical may be further substituted with a variety of substituents to form different thiocarbonyl groups including thioacids, thioamides, thioesters, and thioketones.

“Animal” includes humans, non-human mammals (e.g., non-human primates, rodents, mice, rats, hamsters, dogs, cats, rabbits, cattle, horses, sheep, goats, swine, deer, and the like) and non-mammals (e.g., birds, and the like).

“Bioavailability” as used herein is the fraction or percentage of an administered dose of a drug or pharmaceutical composition that reaches the systemic circulation intact. In general, when a medication is administered intravenously, its bioavailability is 100%. However, when a medication is administered via other routes (e.g., orally), its bioavailability decreases (e.g., due to incomplete absorption and first-pass metabolism). Methods to improve the bioavailability include prodrug approach, salt synthesis, particle size reduction, complexation, change in physical form, solid dispersions, spray drying, and hot-melt extrusion.

“Disease” specifically includes any unhealthy condition of an animal or part thereof and includes an unhealthy condition that may be caused by, or incident to, medical or veterinary therapy applied to that animal, i.e., the “side effects” of such therapy.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means organic or inorganic salts of compounds of the present invention which are pharmaceutically acceptable, as defined above, and which possess the desired pharmacological activity. Such salts include acid addition salts formed with inorganic acids, or with organic acids. Pharmaceutically acceptable salts also include base addition salts which may be formed when acidic protons present are capable of reacting with inorganic or organic bases. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e.,1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Pharmaceutically acceptable carrier” means a non-toxic solvent, dispersant, excipient, adjuvant, or other material which is mixed with the compounds of the present invention in order to form a pharmaceutical composition, i.e., a dose form capable of administration to the patient. Examples of pharmaceutically acceptable carrier includes suitable polyethylene glycol (e.g., PEG400), surfactant (e.g., Cremophor), or cyclopolysaccharide (e.g., hydroxypropyl-β-cyclodextrin or sulfobutyl ether β-cyclodextrins), polymer, liposome, micelle, nanosphere, etc.

“Pharmacophore,” as defined by The International Union of Pure and Applied Chemistry, is an ensemble of steric and electronic features that is necessary to ensure the optimal supramolecular interactions with a specific biological target and to trigger (or block) its biological response. For example, Camptothecin is the pharmacophore of the well known drug topotecan and irinotecan. Mechlorethamine is the pharmacophore of a list of widely used nitrogen mustard drugs like Melphalan, Cyclophosphamide, Bendamustine, and so on.

“Prodrug” means a compound that is convertible in vivo metabolically into an active pharmaceutical according to the present invention. For example, an inhibitor comprising a hydroxyl group may be administered as an ester that is converted by hydrolysis in vivo to the hydroxyl compound.

“Stability” in general refers to the length of time a drug retains its properties without loss of potency. Sometimes this is referred to as shelf life. Factors affecting drug stability include, among other things, the chemical structure of the drug, impurity in the formulation, pH, moisture content, as well as environmental factors such as temperature, oxidization, light, and relative humidity. Stability can be improved by providing suitable chemical and/or crystal modifications (e.g., surface modifications that can change hydration kinetics; different crystals that can have different properties), excipients (e.g., anything other than the active substance in the dosage form), packaging conditions, storage conditions, etc.

“Therapeutically effective amount” of a composition described herein is meant an amount of the composition which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the composition described above may range from about 0.1 mg/kg to about 500 mg/kg, preferably from about 0.2 to about 50 mg/kg. Effective doses will also vary depending on route of administration, as well as the possibility of co-usage with other agents. It will be understood, however, that the total daily usage of the compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

As used herein, the term “treating” refers to administering a compound to a subject that has a neoplastic or immune disorder, or has a symptom of or a predisposition toward it, with the purpose to cure, heal, alleviate, relieve, alter, remedy, ameliorate, improve, or affect the disorder, the symptoms of or the predisposition toward the disorder. The term “an effective amount” refers to the amount of the active agent that is required to confer the intended therapeutic effect in the subject. Effective amounts may vary, as recognized by those skilled in the art, depending on route of administration, excipient usage, and the possibility of co-usage with other agents.

A “subject” refers to a human and a non-human animal. Examples of a non-human animal include all vertebrates, e.g., mammals, such as non-human primates (particularly higher primates), dog, rodent (e.g., mouse or rat), guinea pig, cat, and non-mammals, such as birds, amphibians, reptiles, etc. In a preferred embodiment, the subject is a human. In another embodiment, the subject is an experimental animal or animal suitable as a disease model.

“Combination therapy” includes the administration of the subject compounds of the present invention in further combination with other biologically active ingredients (such as, but not limited to, a second and different antineoplastic agent) and non-drug therapies (such as, but not limited to, surgery or radiation treatment). For instance, the compounds of the invention can be used in combination with other pharmaceutically active compounds, or non-drug therapies, preferably compounds that are able to enhance the effect of the compounds of the invention. The compounds of the invention can be administered simultaneously (as a single preparation or separate preparation) or sequentially to the other therapies. In general, a combination therapy envisions administration of two or more drugs/treatments during a single cycle or course of therapy.

In one embodiment, the compounds of the invention are administered in combination with one or more of traditional chemotherapeutic agents. The traditional chemotherapeutic agents encompass a wide range of therapeutic treatments in the field of oncology. These agents are administered at various stages of the disease for the purposes of shrinking tumors, destroying remaining cancer cells left over after surgery, inducing remission, maintaining remission and/or alleviating symptoms relating to the cancer or its treatment. Examples of such agents include, but are not limited to, alkylating agents such as Nitrogen Mustards (e.g., Bendamustine, Cyclophosphamide, Melphalan, Chlorambucil, Isofosfamide), Nitrosureas (e.g., Carmustine, Lomustine and Streptozocin), ethylenimines (e.g., thiotepa, hexamethylmelanine), Alkylsulfonates (e.g., Busulfan), Hydrazines and Triazines (e.g., Altretamine, Procarbazine, Dacarbazine and Temozolomide), and platinum based agents (e.g., Carboplatin, Cisplatin, and Oxaliplatin); plant alkaloids such as Podophyllotoxins (e.g., Etoposide and Tenisopide), Taxanes (e.g., Paclitaxel and Docetaxel), Vinca alkaloids (e.g., Vincristine, Vinblastine and Vinorelbine); anti-tumor antibiotics such as Chromomycins (e.g., Dactinomycin and Plicamycin), Anthracyclines (e.g., Doxorubicin, Daunorubicin, Epirubicin, Mitoxantrone, and Idarubicin), and miscellaneous antibiotics such as Mitomycin and Bleomycin; anti-metabolites such as folic acid antagonists (e.g., Methotrexate), pyrimidine antagonists (e.g., 5-Fluorouracil, Foxuridine, Cytarabine, Capecitabine, and Gemcitabine), purine antagonists (e.g., 6-Mercaptopurine and 6-Thioguanine) and adenosine deaminase inhibitors (e.g., Cladribine, Fludarabine, Nelarabine and Pentostatin); topoisomerase inhibitors such as topoisomerase I inhibitors(Topotecan, Irinotecan), topoisomerase II inhibitors (e.g., Amsacrine, Etoposide, Etoposide phosphate, Teniposide), and miscellaneous anti-neoplastics such as ribonucleotide reductase inhibitors (Hydroxyurea), adrenocortical steroid inhibitor (Mitotane), anti-microtubule agents (Estramustine), and retinoids (Bexarotene, Isotretinoin, Tretinoin (ATRA).

In one aspect of the invention, the compounds may be administered in combination with one or more targeted anti-cancer agents that modulate protein kinases involved in various disease states. Examples of such kinases may include, but are not limited ABL1, ABL2/ARG, ACK1, AKT1, AKT2, AKT3, ALK, ALK1/ACVRL1, ALK2/ACVR1, ALK4/ACVR1B, ALK5/TGFBR1, ALK6/BMPR1B, AMPK(A1/B1/G1), AMPK(A1/B1/G2), AMPK(A1/B1/G3), AMPK(A1/B2/G1), AMPK(A2/B1/G1), AMPK(A2/B2/G1), AMPK(A2/B2/G2), ARAF, ARK5/NUAK1, ASK1/MAP3K5, ATM, Aurora A, Aurora B , Aurora C, AXL, BLK, BMPR2, BMX/ETK, BRAF, BRK, BRSK1, BRSK2, BTK, CAMK1a, CAMK1b, CAMK1d, CAMK1g , CAMKIIa , CAMKIIb, CAMKIId , CAMKIIg , CAMK4, CAMKK1, CAMKK2, CDC7-DBF4, CDK1-cyclin A, CDK1-cyclin B, CDK1-cyclin E, CDK2-cyclin A, CDK2-cyclin Al, CDK2-cyclin E, CDK3-cyclin E, CDK4-cyclin D1, CDK4-cyclin D3, CDK5-p25, CDK5-p35, CDK6-cyclin D1, CDK6-cyclin D3, CDK7-cyclin H, CDK9-cyclin K, CDK9-cyclin T1, CHK1, CHK2, CK1a1, CK1d, CK1 epsilon , CK1g1, CK1g2, CK1g3, CK2a, CK2a2, c-KIT, CLK1, CLK2, CLK3, CLK4, c-MER, c-MET, COT1/MAP3K8, CSK, c-SRC, CTK/MATK, DAPK1, DAPK2, DCAMKL1, DCAMKL2, DDR1, DDR2, DLK/MAP3K12, DMPK, DMPK2/CDC42BPG, DNA-PK, DRAK1/STK17A, DYRK1/DYRK1A, DYRK1B, DYRK2, DYRK3, DYRK4, EEF2K, EGFR, EIF2AK1, EIF2AK2, EIF2AK3, EIF2AK4/GCN2, EPHA1, EPHA2, EPHA3, EPHA4, EPHA5, EPHA6, EPHA7, EPHA8, EPHB1, EPHB2, EPHB3, EPHB4, ERBB2/HER2, ERBB4/HER4, ERK1/MAPK3, ERK2/MAPK1, ERK5/MAPK7, FAK/PTK2, FER, FES/FPS, FGFR1, FGFR2, FGFR3, FGFR4, FGR, FLT1/VEGFR1, FLT3, FLT4/VEGFR3, FMS, FRK/PTK5, FYN, GCK/MAP4K2, GRK1, GRK2, GRK3, GRK4, GRK5, GRK6, GRK7, GSK3a, GSK3b, Haspin, HCK, HGK/MAP4K4, HIPK1, HIPK2, HIPK3, HIPK4, HPK1/MAP4K1, IGF1R, IKKa/CHUK, IKKb/IKBKB, IKKe/IKBKE, IR, IRAK1, IRAK4, IRR/INSRR, ITK, JAK1, JAK2, JAK3, JNK1, JNK2, JNK3, KDR/VEGFR2, KHS/MAP4K5, LATS1, LATS2, LCK, LCK2/ICK, LKB1, LIMK1, LOK/STK10, LRRK2, LYN, LYNB, MAPKAPK2, MAPKAPK3, MAPKAPK5/PRAK, MARK1, MARK2/PAR-1Ba, MARKS, MARK4, MEK1, MEK2, MEKK1, MEKK2, MEKK3, MELK, MINK/MINK1, MKK4, MKK6, MLCK/MYLK, MLCK2/MYLK2, MLK1/MAP3K9, MLK2/MAP3K10, MLK3/MAP3K11, MNK1, MNK2, MRCKa/, CDC42BPA, MRCKb/, CDC42BPB, MSK1/RPS6KA5, MSK2/RPS6KA4, MSSK1/STK23, MST1/STK4, MST2/STK3, MST3/STK24, MST4, mTOR/FRAP1, MUSK, MYLK3, MYO3b, NEK1, NEK2, NEK3, NEK4, NEK6, NEK7, NEK9, NEK11, NIK/MAP3K14, NLK, OSR1/OXSR1, P38a/MAPK14, P38b/MAPK11, P38d/MAPK13, P38g/MAPK12, P70S6K/RPS6KB1, p70S6Kb/, RPS6KB2, PAK1, PAK2, PAK3, PAK4, PAK5, PAK6, PASK, PBK/TOPK, PDGFRa, PDGFRb, PDK1/PDPK1, PDK1/PDHK1, PDK2/PDHK2, PDK3/PDHK3, PDK4/PDHK4, PHKg1, PHKg2, PI3Ka, (p110a/p85a), PI3Kb, (p110b/p85a), PI3Kd, (p110d/p85a), PI3Kg(p120g), PIM1, PIM2, PIM3, PKA, PKAcb, PKAcg , PKCa, PKCb1, PKCb2, PKCd, PKCepsilon, PKCeta, PKCg , PKCiota, PKCmu/PRKD1, PKCnu/PRKD3, PKCtheta, PKCzeta, PKD2/PRKD2, PKG1a , PKG1b , PKG2/PRKG2, PKN1/PRK1, PKN2/PRK2, PKN3/PRK3, PLK1, PLK2, PLK3, PLK4/SAK, PRKX, PYK2, RAF1, RET, RIPK2, RIPK3, RIPK5, ROCK1, ROCK2, RON/MST1R, ROS/ROS1, RSK1, RSK2, RSK3, RSK4, SGK1, SGK2, SGK3/SGKL, SIK1, SIK2, SLK/STK2, SNARK/NUAK2, SRMS, SSTK/TSSK6, STK16, STK22D/TSSK1, STK25/YSK1, STK32b/YANK2, STK32c/YANK3, STK33, STK38/NDR1, STK38L/NDR2, STK39/STLK3, SRPK1, SRPK2, SYK, TAK1, TAOK1, TAOK2/TAO1, TAOK3/JIK, TBK1, TEC, TESK1, TGFBR2, TIE2/TEK, TLK1, TLK2, TNIK, TNK1, TRKA, TRKB, TRKC, TRPM7/CHAK1, TSSK2, TSSK3/STK22C, TTBK1, TTBK2, TTK, TXK, TYK1/LTK, TYK2, TYRO3/SKY, ULK1, ULK2, ULK3, VRK1, VRK2, WEE1, WNK1, WNK2, WNK3, YES/YES1, ZAK/MLTK, ZAP70, ZIPK/DAPK3, KINASE, MUTANTS, ABL1(E255K), ABL1(F317I), ABL1(G250E), ABL1(H396P), ABL1(M351T), ABL1(Q252H), ABL1(T315I), ABL1(Y253F), ALK (C1156Y), ALK(L1196M), ALK (F1174L), ALK (R1275Q), BRAF(V599E), BTK(E41K), CHK2(I157T), c-Kit(A829P), c-KIT(D816H), c-KIT(D816V), c-Kit(D820E), c-Kit(N822K), C-Kit (T670I), c-Kit(V559D), c-Kit(V559D/V654A), c-Kit(V559D/T670I), C-Kit (V560G), c-KIT(V654A), C-MET(D1228H), C-MET(D1228N), C-MET(F1200I), c-MET(M1250T), C-MET(Y1230A), C-MET(Y1230C), C-MET(Y1230D), C-MET(Y1230H), c-Src(T341M), EGFR(G719C), EGFR(G719S), EGFR(L858R), EGFR(L861Q), EGFR(T790M), EGFR, (L858R,T790M), EGFR(d746-750/T790M), EGFR(d746-750), EGFR(d747-749/A750P), EGFR(d747-752/P753S), EGFR(d752-759), FGFR1(V561M), FGFR2(N549H), FGFR3(G697C), FGFR3(K650E), FGFR3(K650M), FGFR4(N535K), FGFR4(V550E), FGFR4(V550L), FLT3(D835Y), FLT3(ITD), JAK2 (V617F), LRRK2 (G2019S), LRRK2 (12020T), LRRK2 (R1441C), p38a(T106M), PDGFRa(D842V), PDGFRa(T674I), PDGFRa(V561D), RET(E762Q), RET(G691S), RET(M918T), RET(R749T), RET(R813Q), RET(V804L), RET(V804M), RET(Y791F), TIF2(R849W), TIF2(Y897S), and TIF2(Y1108F).

In another aspect of the invention, the subject compounds may be administered in combination with one or more targeted anti-cancer agents that modulate non-kinase biological targets, pathway, or processes. Such targets pathways, or processes include but not limited to heat shock proteins (e.g. HSP90), poly-ADP (adenosine diphosphate)-ribose polymerase (PARP), hypoxia-inducible factors(HIF), proteasome, Wnt/Hedgehog/Notch signaling proteins, TNF-alpha, matrix metalloproteinase, farnesyl transferase, apoptosis pathway (e.g Bcl-xL, Bcl-2, Bcl-w), histone deacetylases (HDAC), histone acetyltransferases (HAT), and methyltransferase (e.g histone lysine methyltransferases, histone arginine methyltransferase, DNA methyltransferase, etc).

In another aspect of the invention, the compounds of the invention are administered in combination with one or more of other anti-cancer agents that include, but are not limited to, gene therapy, RNAi cancer therapy, chemoprotective agents (e.g., amfostine, mesna, and dexrazoxane), drug-antibody conjugate(e.g brentuximab vedotin, ibritumomab tioxetan), cancer immunotherapy such as Interleukin-2, cancer vaccines(e.g., sipuleucel-T) or monoclonal antibodies (e.g., Bevacizumab, Alemtuzumab, Rituximab, Trastuzumab, etc).

In another aspect of the invention, the subject compounds are administered in combination with radiation therapy or surgeries. Radiation is commonly delivered internally (implantation of radioactive material near cancer site) or externally from a machine that employs photon (x-ray or gamma-ray) or particle radiation. Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

In certain embodiments, the compounds of the invention are administered in combination with one or more of radiation therapy, surgery, or anti-cancer agents that include, but are not limited to, DNA damaging agents, antimetabolites, topoisomerase inhibitors, anti-microtubule agents, kinase inhibitors, epigenetic agents, HSP90 inhibitors, PARP inhibitors, BCL-2 inhibitor, drug-antibody conjugate, and antibodies targeting VEGF, HER2, EGFR, CD50, CD20, CD30, CD33, etc.

In certain embodiments, the compounds of the invention are administered in combination with one or more of abarelix, abiraterone acetate, aldesleukin, alemtuzumab, altretamine, anastrozole, asparaginase, bendamustine, bevacizumab, bexarotene, bicalutamide, bleomycin, bortezombi, brentuximab vedotin, busulfan, capecitabine, carboplatin, carmustine, cetuximab, chlorambucil, cisplatin, cladribine, clofarabine, clomifene, crizotinib, cyclophosphamide, dasatinib, daunorubicin liposomal, decitabine, degarelix, denileukin diftitox, denileukin diftitox, denosumab, docetaxel, doxorubicin, doxorubicin liposomal, epirubicin, eribulin mesylate, erlotinib, estramustine, etoposide phosphate, everolimus, exemestane, fludarabine, fluorouracil, fotemustine, fulvestrant, gefitinib, gemcitabine, gemtuzumab ozogamicin, goserelin acetate, histrelin acetate, hydroxyurea, ibritumomab tiuxetan, idarubicin, ifosfamide, imatinib mesylate, interferon alfa 2a, ipilimumab, ixabepilone, lapatinib ditosylate, lenalidomide, letrozole, leucovorin, leuprolide acetate, levamisole, lomustine, mechlorethamine, melphalan, methotrexate, mitomycin C, mitoxantrone, nelarabine, nilotinib, oxaliplatin, paclitaxel, paclitaxel protein-bound particle, pamidronate, panitumumab, pegaspargase, peginterferon alfa-2b, pemetrexed disodium, pentostatin, raloxifene, rituximab, sorafenib, streptozocin, sunitinib maleate, tamoxifen, temsirolimus, teniposide, thalidomide, toremifene, tositumomab, trastuzumab, tretinoin, uramustine, vandetanib, vemurafenib, vinorelbine, zoledronate, radiation therapy, or surgery.

In certain embodiments, the compounds of the invention are administered in combination with one or more anti-inflammatory agent. Anti-inflammatory agents include but are not limited to NSAIDs, non-specific and COX-2 specific cyclooxgenase enzyme inhibitors, gold compounds, corticosteroids, methotrexate, tumor necrosis factor receptor (TNF) receptors antagonists, immunosuppressants and methotrexate. Examples of NSAIDs include, but are not limited to, ibuprofen, flurbiprofen, naproxen and naproxen sodium, diclofenac, combinations of diclofenac sodium and misoprostol, sulindac, oxaprozin, diflunisal, piroxicam, indomethacin, etodolac, fenoprofen calcium, ketoprofen, sodium nabumetone, sulfasalazine, tolmetin sodium, and hydroxychloroquine. Examples of NSAIDs also include COX-2 specific inhibitors such as celecoxib, valdecoxib, lumiracoxib and/or etoricoxib.

In some embodiments, the anti-inflammatory agent is a salicylate. Salicylates include by are not limited to acetylsalicylic acid or aspirin, sodium salicylate, and choline and magnesium salicylates. The anti-inflammatory agent may also be a corticosteroid. For example, the corticosteroid may be cortisone, dexamethasone, methylprednisolone, prednisolone, prednisolone sodium phosphate, or prednisone.

In additional embodiments the anti-inflammatory agent is a gold compound such as gold sodium thiomalate or auranofin.

The invention also includes embodiments in which the anti-inflammatory agent is a metabolic inhibitor such as a dihydrofolate reductase inhibitor, such as methotrexate or a dihydroorotate dehydrogenase inhibitor, such as leflunomide.

Other embodiments of the invention pertain to combinations in which at least one anti-inflammatory compound is an anti-C5 monoclonal antibody (such as eculizumab or pexelizumab), a TNF antagonist, such as entanercept, or infliximab, which is an anti-TNF alpha monoclonal antibody.

In certain embodiments, the compounds of the invention are administered in combination with one or more immunosuppressant agents.

In some embodiments, the immunosuppressant agent is glucocorticoid, methotrexate, cyclophosphamide, azathioprine, mercaptopurine, leflunomide, cyclosporine, tacrolimus, and mycophenolate mofetil, dactinomycin, anthracyclines, mitomycin C, bleomycin, or mithramycin, or fingolimod.

The invention further provides methods for the prevention or treatment of a neoplastic disease, autoimmune and/or inflammatory disease. In one embodiment, the invention relates to a method of treating a neoplastic disease, autoimmune and/or inflammatory disease in a subject in need of treatment comprising administering to said subject a therapeutically effective amount of a compound of the invention. In one embodiment, the invention further provides for the use of a compound of the invention in the manufacture of a medicament for halting or decreasing a neoplastic disease, autoimmune and/or inflammatory disease.

In one embodiment, the neoplastic disease is a B-cell malignancy includes but not limited to B-cell lymphoma, lymphoma (including Hodgkin's lymphoma and non-Hodgkin's lymphoma), hairy cell lymphoma, small lymphocytic lymphoma (SLL), mantle cell lymphoma (MCL), and diffuse large B-cell lymphoma (DLBCL), multiple myeloma, chronic and acute myelogenous leukemia and chronic and acute lymphocytic leukemia.

The autoimmune and/or inflammatory diseases that can be affected using compounds and compositions according to the invention include, but are not limited to allergy, Alzheimer's disease, acute disseminated encephalomyelitis, Addison's disease, ankylosing spondylitis, antiphospholipid antibody syndrome, asthma, atherosclerosis, autoimmune hemolytic anemia, autoimmune hemolytic and thrombocytopenic states, autoimmune hepatitis, autoimmune inner ear disease, bullous pemphigoid, coeliac disease, chagas disease, chronic obstructive pulmonary disease, chronic Idiopathic thrombocytopenic purpura (ITP), churg-strauss syndrome, Crohn's disease, dermatomyositis, diabetes mellitus type 1, endometriosis, Goodpasture's syndrome (and associated glomerulonephritis and pulmonary hemorrhage), graves' disease, guillain-barre syndrome, hashimoto's disease, hidradenitis suppurativa, idiopathic thrombocytopenic purpura, interstitial cystitis, irritable bowel syndrome, lupus erythematosus, morphea, multiple sclerosis, myasthenia gravis, narcolepsy, neuromyotonia, Parkinson's disease, pemphigus vulgaris, pernicious anaemia, polymyositis, primary biliary cirrhosis, psoriasis, psoriatic arthritis, rheumatoid arthritis, schizophrenia, septic shock, scleroderma, Sjogren's disease, systemic lupus erythematosus (and associated glomerulonephritis), temporal arteritis, tissue graft rejection and hyperacute rejection of transplanted organs, vasculitis (ANCA-associated and other vasculitides), vitiligo, and wegener's granulomatosis.

It should be understood that the invention is not limited to the particular embodiments shown and described herein, but that various changes and modifications may be made without departing from the spirit and scope of the invention as defined by the claims.

The compounds according to the present invention may be synthesized according to a variety of reaction schemes. Necessary starting materials may be obtained by standard procedures of organic chemistry. The compounds and processes of the present invention will be better understood in connection with the following representative synthetic schemes and examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, and/or methods of the invention may be made without departing from the spirit of the invention and the scope of the appended claims.

The typical starting material

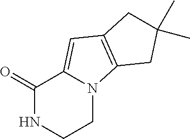

(CAS 1346674-23-4) is commercially available. However, the reported route, e.g. in WO 2013067274, to this intermediate entails at least 7 synthetic steps. The synthesis not only is long, it also includes several reagents and solvents that are toxic or hazardous and present environmental liabilities. We describe herein in Scheme I a new, more efficient, and cost-effective route (three synthetic steps) focused on the use of sustainable chemistry:

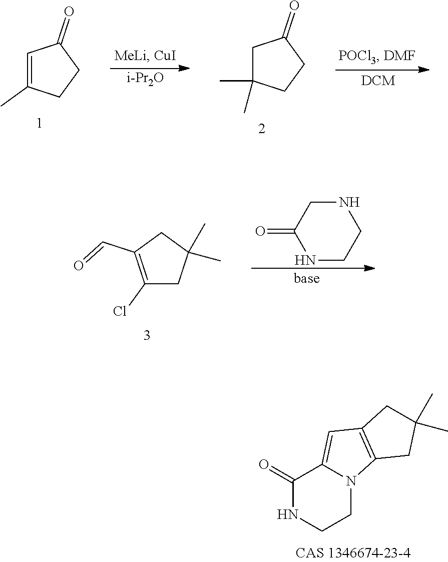

In Scheme I, the starting material 3-methylcyclopent-2-en-1-one was converted to 3,3-dimethylcyclopentan-1-one by standard organic reaction with high yield, which can further be converted to intermediate 3. Finally, intermediate 3 can react with piperazin-2-one to yield the target molecule of CAS 1346674-23-4.

The intermediate

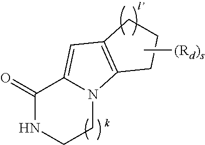

in which each of k, r, and s, independently, is 0, 1, 2, or 3, can be made by the method similar to Scheme I, by using different starting material and reagents.

The intermediate

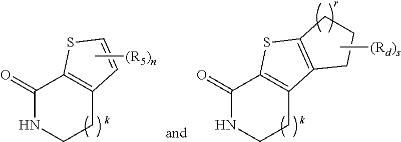

in which each of k, r, and s, independently, is 0, 1, 2, or 3, can be made by the method similar to those disclosure in the WO/2013/067260, WO/2013/067274, WO/2013/067277, WO/2015/000949.

The intermediate

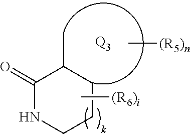

can be made by the method similar to Scheme I, by using different starting material and reagents, or by the standard organic reactions.

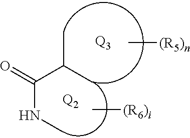

The intermediate can be made by the method similar to Scheme 1 by using different starting material and reagents, or by the standard organic reactions.

The intermediate

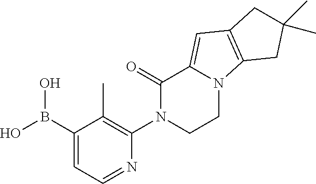 [SMILES: Cc1c(B(O)O)ccnc1N1CCn2c(cc3c2CC(C)(C)C3)C1=O]

can be made by the Scheme 1 described below.

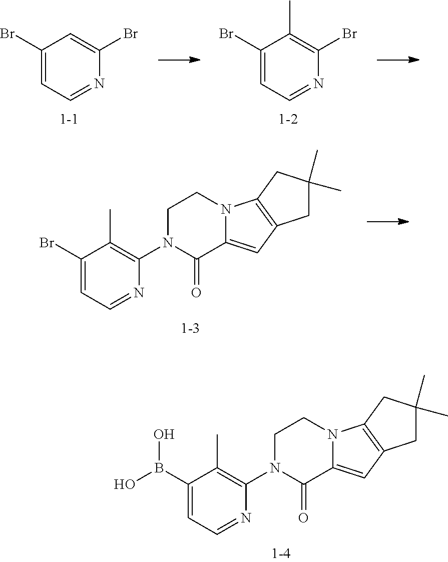

In Scheme 1, the starting material 2,4-dibromopyridine was converted to intermediate 1-2 by standard organic reaction with high yield, which can further react with 7,7-dimethyl-3,4,7,8-tetrahydro-2H-cyclopenta[4,5]pyrrolo[1,2-a]pyrazin-1(6H)-one (CAS 1346674-23-4) to afford the intermediate 1-3. Finally, 1-3 can be converted to target borate intermediate 1-4 by standard organic reaction.

The intermediate

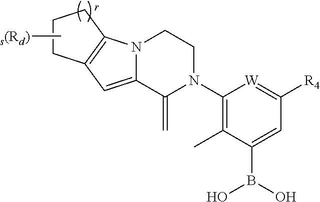

can be made by the method similar to Scheme 1, by using different starting material and reagents, or by the standard organic reactions.

The intermediate

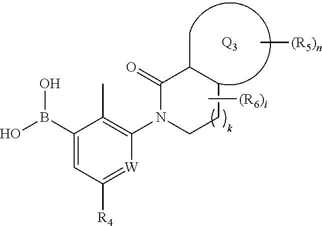

can be made by the method similar to Scheme 1, by using different starting material and reagents, or by the standard organic reactions.

The intermediate

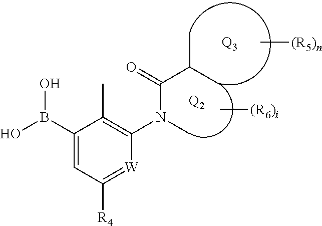

can be made by the method similar to Scheme 1 by using appropriate starting material and reagents, or by the standard organic reactions.

The intermediate

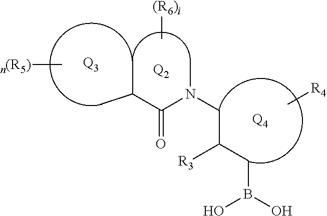

can be made by the method similar to Scheme 1 by using appropriate starting material and reagents, or by the standard organic reactions.

The intermediate

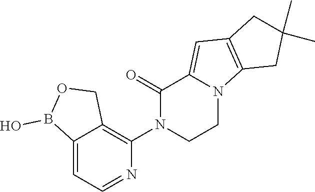 [SMILES: CC1(C)Cc2cc3n(c2C1)CCN(c1nccc2c1COB2O)C3=O]

can be made by the Scheme 2 described below.

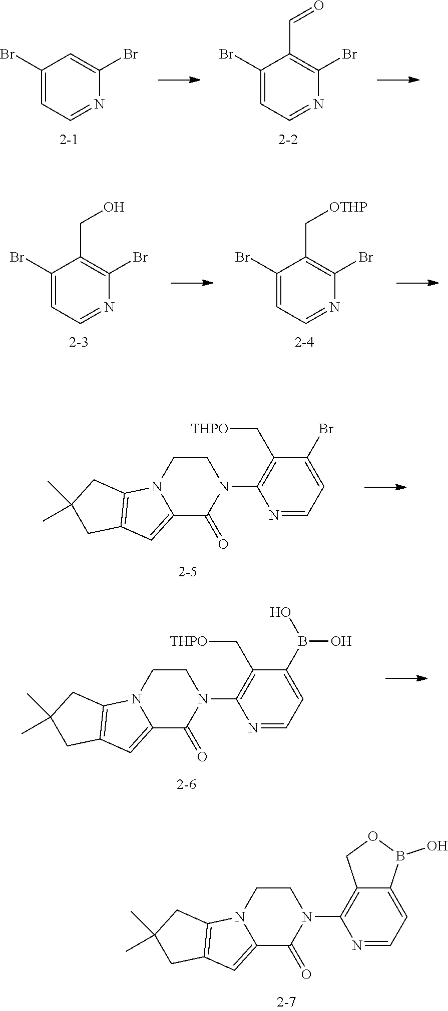

In Scheme 2, the starting material 2,4-dibromopyridine was converted to 2,4-dibromonicotinaldehyde by standard organic reaction with high yield, which can further be reduced to the alcohol intermediate 2-3. After that, the OH group of intermediate 2-3 can be protected by the THP to form the intermediate 2-4, which can react with 7,7-dimethyl-3,4,7,8-tetrahydro-2H-cyclopenta[4,5]pyrrolo[1,2-a]pyrazin-1(6H)-one (CAS 1346674-23-4) to afford the intermediate 2-5. Next, intermediate 2-5 can be converted to the intermediate 2-6, which can undergo a ring closure reaction to yield the intermediate 2-7.

The intermediate

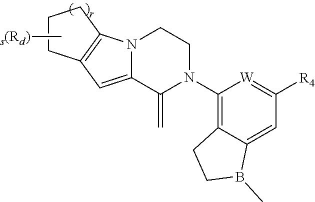

can be made by the method similar to Scheme 2, by using different starting material and reagents, or by the standard organic reactions.

The intermediate

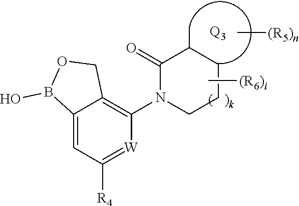

can be made by the method similar to Scheme 1, by using different starting material and reagents, or by the standard organic reactions.

The intermediate

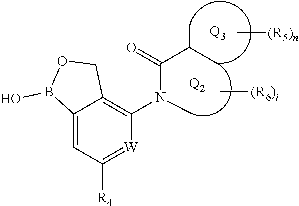

can be made by the method similar to Scheme 2 by using appropriate starting material and reagents, or by the standard organic reactions.

An typical approach to synthesize compounds of

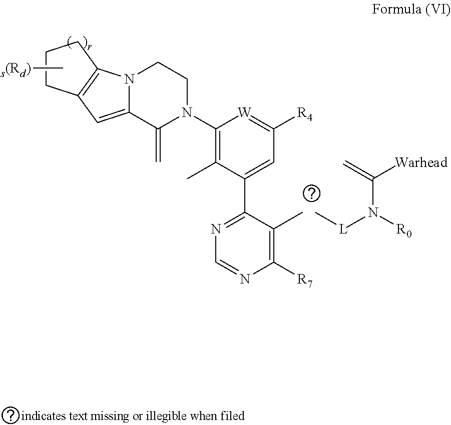

(wherein V is N and R₃ is —CH₃) is described in Scheme A. R₇, W, R₄, R_{d}, r, s, L, R₀, and warhead in general Scheme A are the same as those described in the Summary section above.

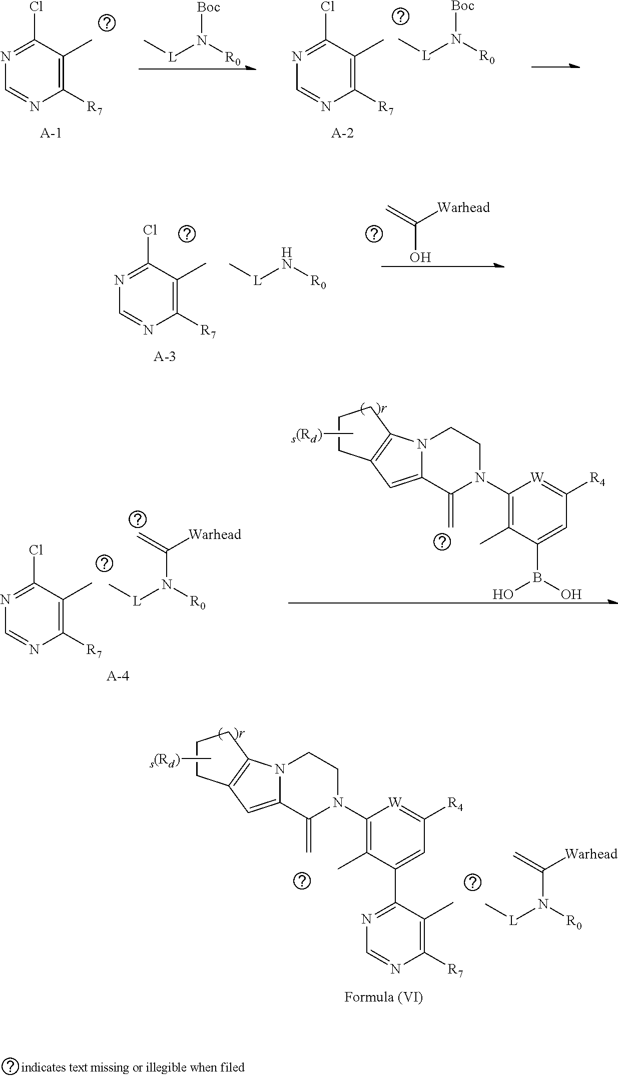

In Scheme A, the appropriate starting material A-1 can be prepared by standard organic reaction. A-1 can react with appropriate alcohol to form the intermediate A-2 followed by a de-Boc process to yield A-3. The amide couping of A-3 and appropriate carboxylic acid will form A-4 which can couple with the appropriate borate to yield the target compounds.

An typical approach to synthesize compounds of

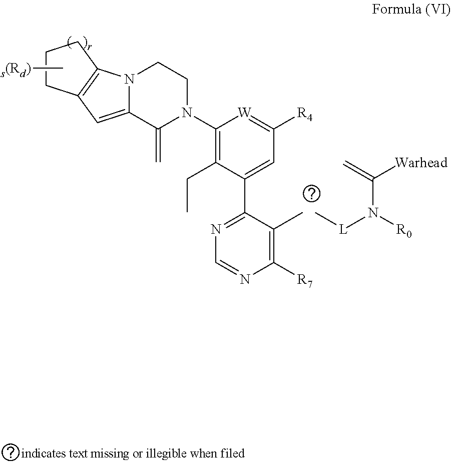

(wherein V is N and R₃ is —CH₂OH) is described in Scheme B. R₇, W, R₄, R_{d}, r, s, L, R₀, and warhead in general Scheme B are the same as those described in the Summary section above.

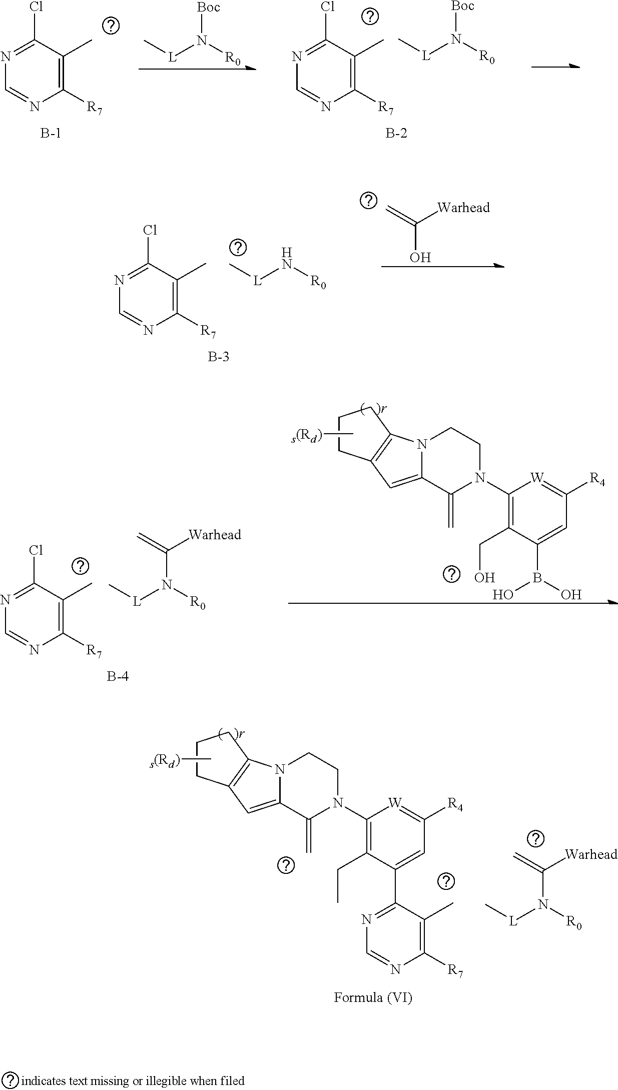

In Scheme B, the appropriate starting material B-1 can be prepared by standard organic reaction. B-1 can react with appropriate alcohol to form the intermediate B-2 followed by a de-Boc process to yield B-3. The amide couping of B-3 and appropriate carboxylic acid will form B-4 which can couple with the appropriate borate to yield the target compounds.

The compounds

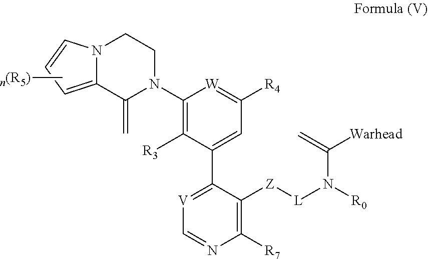

can be made by the method similar to Scheme A and B, by using different starting material, intermediates, and reagents.

The compounds

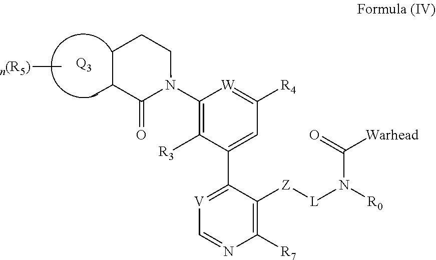

can be made by the method similar to Scheme A and B, by using different starting material, intermediates, and reagents.

The compounds

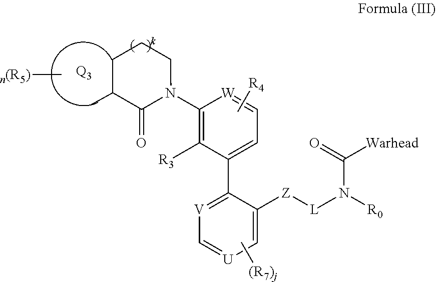

can be made by the method similar to Scheme A and B, by using different starting material, intermediates, and reagents.

The compounds

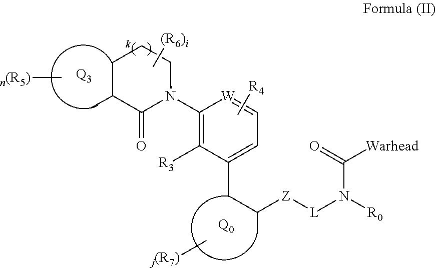

can be made by the method similar to Scheme A and B, by using different starting material, intermediates, and reagents.

The compounds

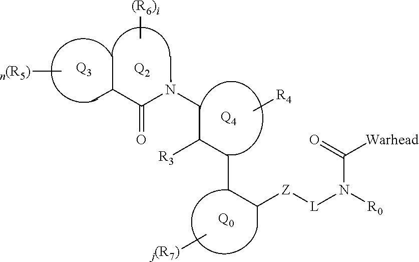

can be made by the method similar to Scheme A and B, by using different starting material, intermediates, and reagents.

An improved approach to synthesize compounds of

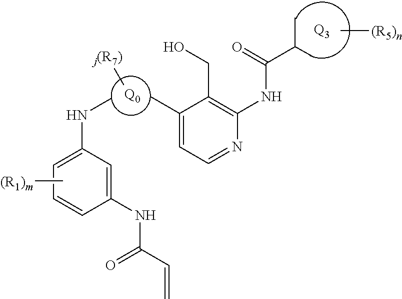

is described in Scheme 1. Q₀, Q₃, R₁, R₅, R₇, j, m, and n in general Scheme 1 are the same as those described in the Summary section above.

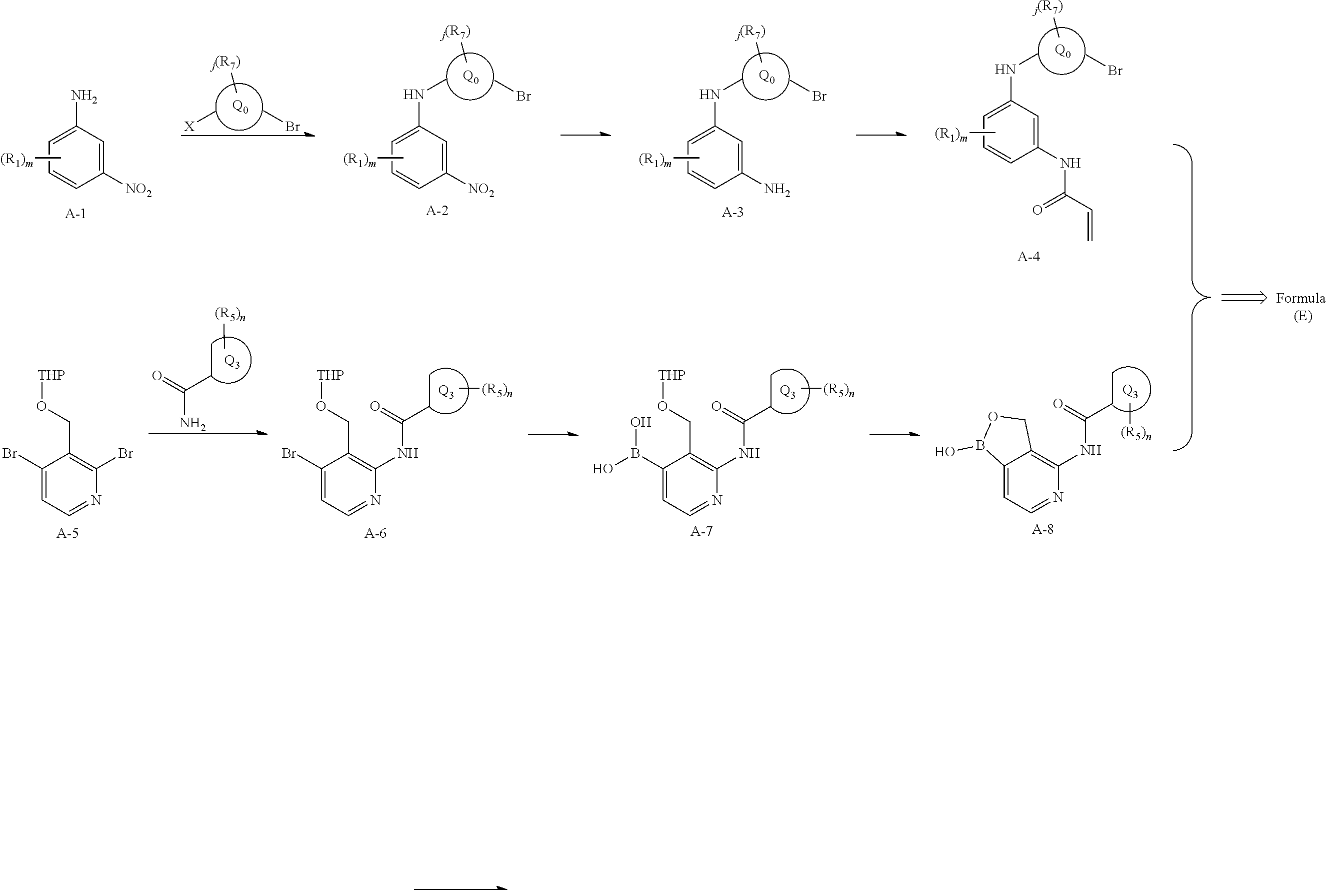

In Scheme 1, the appropriate starting material 3-nitroaniline A-1 can react with appropriate halo subsubstited QO to yield the nitro intermediate A-2, which can be reduced to the amine intermediate A-3. A-3 can react appropriate acryloyl chloride to afford A-4. Meanwhile, the appropriate starting material A-5, which was described in PCT/US2020/019478, can react with appropriate amide to afford the intermediate A-6, Next, intermediate A-6 can be converted to the borate A-7, which can undergo a ring closure reaction to yield the intermediate A-7. Finally, the couping of A-4 and A-8 will yield the target compounds of Formula(E).

The Formula (D) compounds

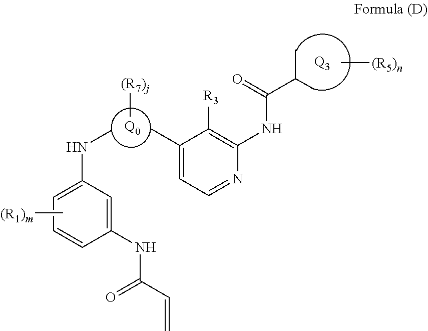

can be made by the method similar to Scheme A by using different starting material, intermediates, and reagents.

The Formula (C) compounds

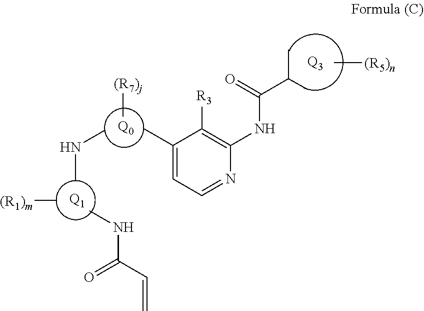

can be made by the method similar to Scheme 1, by using different starting material, intermediates, and reagents.

The Formula (B) compounds

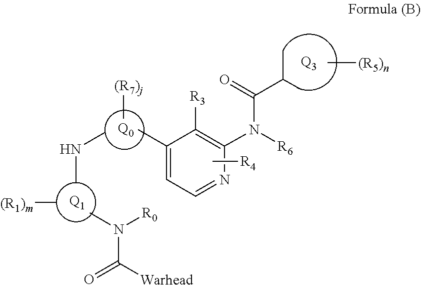

can be made by the method similar to Scheme 1, by using different starting material, intermediates, and reagents.

The Formula (A) compounds

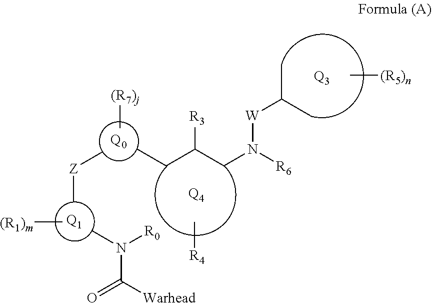

can be made by the method similar to Scheme A and B, by using different starting material, intermediates, and reagents.

The compounds and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Where NMR data are presented, ¹H spectra were obtained on XL400 (400 MHz) and are reported as ppm down field from Me₄Si with number of protons, multiplicities, and coupling constants in Hertz indicated parenthetically. Where HPLC data are presented, analyses were performed using an Agilent 1100 system. Where LC/MS data are presented, analyses were performed using an Applied Biosystems API-100 mass spectrometer and Shimadzu SCL-10A LC column:

### EXAMPLE 1

### Preparation of 4-tert-butyl-N-[3-(hydroxymethyl)-4-[4-methyl-6-([4-[(2S)-2-methyl-4-(oxan-4-yl)piperazin-1-yl]-3-(prop-2-enamido)phenyl]amino)-5-oxopyrazin-2-yl]pyridin-2-yl]benzamide

Synthesis of 2,4-dibromopyridine-3-carbaldehyde: Into a 10000-mL 4-necked round-bottom flask, was placed 2,4-dibromopyridine (500.00 g, 2.11 mol, 1.00 equiv), THF (5000.00 mL). This was followed by the addition of LDA (2M in hexane, 1.58 L, 1.5 equiv) dropwise with stirring at −78 degrees C. The resulting solution was stirred for 1 h at −78 degrees C. Then add DMF (200 g, 2.74 mol, 1.3 equiv) by dropwise with stirring at −78 degrees C. The resulting solution was stirred for 1 h at −78 degrees C. The reaction was then quenched by the addition of 5000 mL of aq. NH₄CL/HOAc(1:1). The resulting solution was extracted with 3×5000 mL of ethyl acetate concentrated. The residue was applied onto a silica gel column and eluted with ethyl acetate/petroleum ether (0:1-1:1). This resulted in 450 g (Y=80%) of 2,4-dibromopyridine-3-carbaldehyde as a white solid. LC-MS: (ES, m/z): M+1: 264

Synthesis of (2,4-dibromopyridin-3-yl)methanol: Into a 10000-mL 4-necked round-bottom flask, was placed 2,4-dibromopyridine-3-carbaldehyde (450 g, 1.7mol, 1.00 equiv), EtOH (4500.00 mL). This was followed by the addition of NaBH₄ (65 g, 1.7 mol, equiv), in portions at 0 degrees C. The resulting solution was stirred for 3 h at room temperature. The reaction was then quenched by the addition of 3000 mL of water. The resulting solution was extracted with 3×3000 mL of ethyl acetate concentrated. The residue was applied onto a silica gel column and eluted with ethyl acetate/petroleum ether (1:1). The collected fractions were combined and concentrated. This resulted in 500 g (crude, 90%) of (2,4-dibromopyridin-3-yl)methanol as a light yellow solid. LC-MS: (ES, m/z): M+1: 266

Synthesis of 2,4-dibromo-3-[(oxan-2-yloxy)methyl]pyridine: Into a 10-L 4-necked round-bottom flask, was placed (2,4-dibromopyridin-3-yl)methanol (500 g, 1.89mol, 1.00 equiv), DCM (5 L), PPTS (47.358 g, 188.68 mmol, 0.10 equiv), DHP (237.73 g, 2.83 mol, 1.50 equiv). The resulting solution was stirred for overnight at 45 degrees C. in an oil bath. The reaction was then quenched by the addition of 3 L of water. The resulting solution was extracted with 3×5 L of dichloromethane concentrated. The residue was applied onto a silica gel column and eluted with ethyl acetate/petroleum ether (1:1). The collected fractions were combined and concentrated. This resulted in 560 g (Y=97.4%) of 2,4-dibromo-3-[(oxan-2-yloxy)methyl]pyridine as colorless oil. LC-MS: (ES, m/z): M+1: 350

Synthesis of 3,5-dibromo-1H-pyrazin-2-one: Into a 20 L round-bottom flask purged and maintained with an inert atmosphere of nitrogen, was placed 3,5-dibromopyrazin-2-amine (1000.00 g), AcOH (6L). This was followed by the addition of H₂SO₄ (1.5 L) dropwise with stirring at 10 degrees C. To this was added a solution of NaNO₂ (550.00 g, 2 eq) in 500 mL H₂O dropwise with stirring at 10 degrees C. The resulting solution was stirred for 2 h at room temperature. The resulting solution was diluted with 10 L of cold H₂O. The resulting solution was extracted with 3×3 L of ethyl acetate. The resulting mixture was washed with 3 L of NaCl(aq). The mixture was dried over anhydrous sodium sulfate and concentrated. This resulted in 1020 g of 3,5-dibromo-1H-pyrazin-2-one as a brown solid.

Synthesis of 3,5-dibromo-1-methylpyrazin-2-one and 5-bromo-3-iodo-1-methylpyrazin-2-one: Into a 20-L round-bottom flask, was placed 3,5-dibromo-1H-pyrazin-2-one (1020.00 g, 4047.6 mmol, 1.00 equiv), acetone (7 L). This was followed by the addition of K₂CO₃ (782 g, 5666.7 mmol, 1.40 equiv) in several batches at 18 degrees C. To this was added CH₃I (747.19 g, 5261.9 mmol, 1.30 equiv) in dropwise at 18 degrees C. The resulting solution was stirred for 3 h at room temperature. The resulting solution was diluted with 10 L of H₂O. The resulting solution was extracted with 3×3 L of ethyl acetate. The resulting mixture was washed with 2×1 L of H₂O. The resulting mixture was washed with 1×1 L of NaCl(aq). The mixture was dried over anhydrous sodium sulfate and concentrated. This resulted in 1030 g of 3,5-dibromo-1-methylpyrazin-2-one as a solid. H-NMR (300 MHz, DMSO, ppm): δ 8.17 (s, 2H), 8.09 (s, 1H), 3.46 (s, 6H), 3.44 (s, 3H).

Synthesis of 5-bromo-3-[(4-fluoro-3-nitrophenyl)amino]-1-methylpyrazin-2-one: Into a 10 L round-bottom flask, was placed 4-fluoro-3-nitroaniline (586.47 g, 3.759mol, 1.00equiv), 3,5-dibromo-1-methylpyrazin-2-one (1000 g, 3.759mol, 1.00equiv), NMP(3000 ml). The resulting solution was stirred for 1 h at 140 degrees C. in an oil bath. The resulting solution was diluted with 3 L of H₂O. The solids were collected by filtration. The solids washed by EA(2×1 L). This resulted in 980 g (90%) of 5-bromo-3-[(4-fluoro-3-nitrophenyl) amino]-1-methylpyrazin-2-one as a brown solid. LC-MS: (ES, m/z): M+1: 343/345.

Synthesis of tert-butyl (3S)-4-[4-[(6-bromo-4-methyl-3-oxopyrazin-2-yl)amino]-2-nitrophenyl]-3-methylpiperazine-1-carboxylate: Into a 10 L round-bottom flask, was placed 5-bromo-3-[(4-fluoro-3-nitrophenyl)amino]-1-methylpyrazin-2-one (1000 g, 2923.9mmol, 1.00equiv), NMP (4000.00 mL), tert-butyl (3S)-3-methylpiperazine-1-carboxylate (701.7 g, 3508.7mmol, 1.20 equiv), DIEA (1131.55 g, 8771.7mmol, 3.00equiv). The resulting solution was stirred for 68h at 135° C. in an oil bath. The resulting solution was diluted with 10000 mL of H₂O. The solids were collected by filtration. This resulted in 1200 (crude) g of tert-butyl (3S)-4-[4-[(6-bromo-4-methyl-3-oxopyrazin-2-yl)amino]-2-nitrophenyl]-3-methylpiperazine-1-carboxylate as a brown solid. LC-MS: (ES, m/z): M+1: 523.

Synthesis of 5-bromo-1-methyl-3-([4-[(2S)-2-methylpiperazin-1-yl]-3-nitrophenyl]amino)pyrazin-2-one: Into a 20 L round-bottom flask, was placed tert-butyl (3S)-4-[4-[(6-bromo-4-methyl-3-oxopyrazin-2-yl)amino]-2-nitrophenyl]-3-methylpiperazine-1-carboxylate (1200 g, 1 equiv), dioxane(3000ml), HCl (4M)in 1,4-dioxane (3000.00 mL). The resulting solution was stirred for 13 h at room temperature. The solids were collected by filtration. Filter cake was washed by EA. The filter cake was diluted with 300 mL of H₂O. The pH value of the solution was adjusted to 8 with NaHCO₃. The solids were collected by filtration. This resulted in 900 g (87.8%) of 5-bromo-1-methyl-3-([4-[(2S)-2-methylpiperazin-1-yl]-3-nitrophenyl]amino)pyrazin-2-one as a red solid. LC-MS: (ES, m/z): M+1: 423.

Synthesis of 5-bromo-1-methyl-3-([4-[(2S)-2-methyl-4-(oxan-4-yl)piperazin-1-yl]-3-nitrophenyliamino)pyrazin-2-one: Into a 20 L round-bottom flask, was placed 5-bromo-1-methyl-3-([4-[(2S)-2-methylpiperazin-1-yl]-3-nitrophenyl]amino)pyrazin-2-one (900 g, 2.20 mol, 1.00 equiv), 4H-pyran-4-one, tetrahydro-(330.5 g, 3.30 mol, 1.50 equiv), DCM (9000 ml), AcOH (0.2 eq), NaBH(AcO)₃(1168 g, 5.50 mol, 2.50 equiv). The resulting solution was stirred for 14 h at 30° C. in an oil bath. The resulting mixture was quenched with water and extracted by DCM, dried by Na₂SO₄, the resulting mixture was concentrated. This resulted in 540 g of 5-bromo-1-methyl-3-(4-[(2S)-2-methyl-4-(oxan-4-yl)piperazin-1-yl]-3-nitrophenyl]amino)pyrazin-2-one as a white solid LC-MS: (ES, m/z): M+1: 507.

Synthesis of 3-([3-amino-4-[(2S)-2-methyl-4-(oxan-4-yl)piperazin-1-yl]phenyl]amino)-5-bromo-1-methylpyrazin-2-one: Into a 3000-mL 4-necked round-bottom flask, was placed 5-bromo-1-methyl-3-([4-[(2S)-2-methyl-4-(oxan-4-yl)piperazin-1-yl]-3-nitrophenyl]amino)pyrazin-2-one (270.00 g, 0.533 mol, 1.00 equiv), EtOH (1350.00 mL), H₂O (450.00 mL), Fe (119.53 g, 2.134 mol, 4.00 equiv), NH₄Cl (230.47 g, 4.268mol, 8.00 equiv). The resulting solution was stirred for 1.5 hr at 80 degrees C. in an oil bath. The solids were filtered out. The resulting mixture was concentrated. This resulted in 250 g (90.5%) of 3-([3-amino-4-[(2S)-2-methyl-4-(oxan-4-yl)piperazin-1-yl]phenyl]amino)-5-bromo-1-methylpyrazin-2-one as a yellow solid. LC-MS: (ES, m/z): M+1: 477

Synthesis of N-[5-[(6-bromo-4-methyl-3-oxopyrazin-2-yl)amino]-2-[(2S)-2-methyl-4-(oxan-4-yl)piperazin-1-yl]phenyl]prop-2-enamide: Into a 5000-mL 4-necked round-bottom flask, was placed 3-([3-amino-4-[(2S)-2-methyl-4-(oxan-4-yl)piperazin-1-yl]phenyl]amino)-5-bromo-1-methylpyrazin-2-one (250 g, 0.525 mol, 1.00 equiv), DCM (2500 mL), DIEA (135.5 g, 1.050 mol, 2.00 equiv). This was followed by the addition of acryloyl chloride (47.3g, 0.525mol, 1.00 equiv) dropwise with stirring at 0 degrees C. The resulting solution was stirred for 2 hr at room temperature. The resulting mixture was concentrated. The residue was applied onto a silica gel column with dichloromethane/methanol (10:1). The collected fractions were combined and concentrated. This resulted in 180 g (92% purity) of N-[5-[(6-bromo-4-methyl-3-oxopyrazin-2-yl)amino]-2-[(2S)-2-methyl-4-(oxan-4-yl)piperazin-1-yl]phenyl]prop-2-enamide as a yellow solid. LC-MS: (ES, m/z): M+1: 531.

Synthesis of 4-tert-butylbenzamide: Into a 500 -mL round-bottom flask, was placed 4-tert-butylbenzoic acid (8.00 g, 44.886 mmol, 1.00 equiv), THF (100.00 mL), NH₄Cl (6.15 g, 114.908 mmol, 2.56 equiv), TEA (22.71 g, 224.430 mmol, 5.00 equiv), HATU (20.48 g, 53.863 mmol, 1.20 equiv). The resulting solution was stirred overnight at room temperature. The reaction was then quenched by the addition of 500 mL of water. The resulting solution was extracted with 3×200 mL of ethyl acetate and the organic layers combined. The resulting mixture was washed with 1×600 mL of brine. The mixture was dried over anhydrous sodium sulfate and concentrated under vacuum. The crude product was re-crystallized from PE: EA in the ratio of 3:1. The solids were collected by filtration. This resulted in 5.9 g (74.16%) of 4-tert-butylbenzamide as a white solid. ¹H-NMR (300 MHz, Chloroform-d, ppm) δ 7.81-7.74 (m, 2H), 7.52-7.45 (m, 2H), 6.11 (s, 2H), 1.36 (s, 9H).

Synthesis of N-[4-bromo-3-[(oxan-2-yloxy)methyl]pyridin-2-yl]-4-tert-butylbenzamide: Into a 250-mL round-bottom flask purged and maintained with an inert atmosphere of nitrogen, was placed 2,4-dibromo-3-[(oxan-2-yloxy)methyl]pyridine (3.94 g, 11.224 mmol, 1.00 equiv), toluene (50.00 mL), 4-tert-butylbenzamide (2.00 g, 11.284 mmol, 1.01 equiv), K₂CO₃ (2.34 g, 16.931 mmol, 1.51 equiv), Xantphos (1.31 g, 2.264 mmol, 0.20 equiv), Pd(OAc)₂ (253.00 mg, 1.127 mmol, 0.10 equiv). The resulting solution was stirred for 2 hr at 100 degrees C. in an oil bath. The reaction mixture was cooled to room temperature. The resulting solution was diluted with 300 mL of water. The resulting solution was extracted with 3×100 mL of ethyl acetate and the organic layers combined. The resulting mixture was washed with 1×500 mL of brine. The mixture was dried over anhydrous sodium sulfate and concentrated under vacuum. The residue was applied onto a silica gel column with ethyl acetate/petroleum ether (1:2). The crude product was re-crystallized from PE: EA in the ratio of 10:1. The solids were collected by filtration. This resulted in 2.6 g (51.78%) of N-[4-bromo-3-[(oxan-2-yloxy)methyl]pyridin-2-yl]-4-tert-butylbenzamide as a light yellow solid. LC-MS: (ES, m/z): M+1: 447/449.

Synthesis of 2-(4-tert-butylbenzamido)-3-[(oxan-2-yloxy)methyl]pyridin-4-ylboronic acid: Into a 40-mL vial purged and maintained with an inert atmosphere of nitrogen, was placed N-[4-bromo-3-[(oxan-2-yloxy)methyl]pyridin-2-yl]-4-tert-butylbenzamide (800.00 mg, 1.788 mmol, 1.00 equiv), dioxane (10.00 mL), bis(pinacolato)diboron (1.14 g, 4.489 mmol, 2.51 equiv), KOAc (526.00 mg, 5.360 mmol, 3.00 equiv), Pd(dppf)Cl₂·CH₂Cl₂ (146.00 mg, 0.179 mmol, 0.10 equiv). The resulting solution was stirred overnight at 100 degrees C. in an oil bath. The reaction mixture was cooled to room temperature. The solids were filtered out. The resulting mixture was concentrated under vacuum. The crude product was purified by Flash-Prep-HPLC with the following conditions (IntelFlash-1): Column, C18 reversed phase column; mobile phase, H₂O and CH₃CN; Detector, 220 nm. This resulted in 350 mg (47.47%) of 2-(4-tert-butylbenzamido)-3-[(oxan-2-yloxy)methyl]pyridin-4-ylboronic acid as a white solid. LC-MS: (ES, m/z): M+1: 413

Synthesis of 4-tert-butyl-N-[1-hydroxy-3H-[1,2]oxaborolo[4,3-c]pyridin-4-yl]benzamide: Into a 100-mL round-bottom flask, was placed 2-(4-tert-butylbenzamido)-3-[(oxan-2-yloxy)methyl]pyridin-4-ylboronic acid (350.00 mg, 0.849 mmol, 1.00 equiv), HCl (gas) in 1,4-dioxane (4N, 10.00 mL). The resulting solution was stirred for 1 hr at room temperature. The resulting mixture was concentrated under vacuum. This resulted in 250 mg (94.95%) of 4-tert-butyl-N-[1-hydroxy-3H-[1,2]oxaborolo[4,3-c]pyridin-4-yl]benzamide as a yellow solid. LC-MS: (ES, m/z): M+1: 311.

Synthesis of 4-tert-butyl-N-[3-(hydroxymethyl)-4-[4-methyl-6-([4-[(2S)-2-methyl-4-(oxan-4-yl)piperazin-1-yl]-3-(prop-2-enamido)phenyl]amino)-5-oxopyrazin-2-yl]pyridin-2-yl]benzamide: Into a 40-mL vial purged and maintained with an inert atmosphere of nitrogen, was placed N-[5-[(6-bromo-4-methyl-3-oxopyrazin-2-yl)amino]-2-[(2S)-2-methyl-4-(oxan-4-yl)piperazin-1-yl]phenyl]prop-2-enamide (191.00 mg, 0.359 mmol, 1.00 equiv), dioxane (5.00 mL), H₂O (0.5 mL), 4-tert-butyl-N-[1-hydroxy-3H-[1,2]oxaborolo[4,3-c]pyridin-4-yl]benzamide (222.94 mg, 0.719 mmol, 2.00 equiv), K₂CO₃ (249.00 mg, 1.802 mmol, 5.01 equiv), Xphos Pd G3 (61.00 mg, 0.072 mmol, 0.20 equiv). The resulting solution was stirred for 1 hr at 100 degrees C. in an oil bath. The reaction mixture was cooled to room temperature. The solids were filtered out. The resulting mixture was concentrated under vacuum. The residue was applied onto a silica gel column with dichloromethane/methanol (19:1). The crude product was purified by Prep-HPLC with the following conditions (2#SHIMADZU (HPLC-01)): Column, XBridge Prep C18 OBD Column, 5 um, 19*150 mm; mobile phase, Water (0.05% NH₃·H₂O) and ACN (47% Phase B up to 61% in 7 min); Detector, UV. This resulted in 35 mg (13.25%) of 4-tert-butyl-N-[3-(hydroxymethyl)-4-[4-methyl-6-([4-[(2S)-2-methyl-4-(oxan-4-yl)piperazin-1-yl]-3-(prop-2-enamido)phenyl]amino)-5-oxopyrazin-2-yl]pyridin-2-yl]benzamide as a white solid. LC-MS(ES, m/z): M+1: 735; H-NMR (300 MHz, DMSO-d₆, ppm) δ 9.09 (d, J=12.0 Hz, 2H), 8.53 (d, J=2.5 Hz, 1H), 8.00 (d, J=5.2 Hz, 1H), 7.85 (td, J=8.4, 2.2 Hz, 3H), 7.53 (d, J=8.5 Hz, 2H), 7.23 (s, 1H), 6.75-6.65 (m, 2H), 6.54 (dd, J=16.9, 10.2 Hz, 1H), 6.23 (d, J=16.8 Hz, 1H), 6.07 (s, 2H), 5.77 (d, J=10.3 Hz, 1H), 5.37 (s, 2H), 3.91 (d, J=11.0 Hz, 2H), 3.49 (s, 3H), 3.30-3.24 (m, 2H), 2.90-2.63 (m, 3H), 2.57 (s, 1H), 2.47-2.31 (m, 3H), 2.08 (t, J=9.9 Hz, 1H), 1.74 (d, J=12.5 Hz, 2H), 1.41 (d, J=12.9 Hz, 2H), 1.32 (s, 9H), 0.60 (d, J=6.1 Hz, 3H).

### EXAMPLE 2

### Preparation of (S)-2-(3-(5-((1-acryloylpyrrolidin-2-yl)methoxy)-6-aminopyrimidin-4-yl)-5-fluoro-2-methylphenyl)-7,7-dimethyl-3,4,7,8-tetrahydro-2H-cyclopenta[4,5]pyrrolo[1,2-a]pyrazin-1(6H)-one

Synthesis of [(3,3-dimethylcyclopent-1-en-1-yl)oxy]trimethylsilane: Into a 20-L 4-necked round-bottom flask purged and maintained with an inert atmosphere of nitrogen, was placed CuCl (49.5 g, 500 mmol, 0.05equiv), LiCl (42.4 g, 1000 mmol, 0.10 equiv), THF (6 L). This was followed by the addition of 2-cyclopenten-1-one, 3-methyl- (960.00 g, 10mol, 1.00 equiv) at −5 to 5° C. To this was added TMSCl (1140.3 g, 10.5mol, 1.05 equiv) dropwise with stirring at −5 to 5° C. To the mixture was added MeMgCl (4000 mL, 12 mol, 1.2 equiv) dropwise with stirring at −5 to 5 ° C. The resulting solution was stirred for 2 h at −5 to 5° C. in an ice/salt bath. The reaction was then quenched by the addition of 82 g of MeOH. The resulting solution was diluted with 10 L of NH₄Cl. The solids were filtered out. The resulting solution was extracted with 3×10 L of petroleum ether dried over anhydrous sodium sulfate and concentrated. This resulted in 1730 g (crude) of [(3, 3-dimethylcyclopent-1-en-1-yl)oxy]trimethylsilane as yellow oil. GC-MS: (ES, m/z): M: 184.

Synthesis of 3,3-dimethylcyclopentanone: Into a 20-L 4-necked round-bottom flask, was placed [(3,3-dimethylcyclopent-1-en-1-yl)oxy]trimethylsilane (1730.00 g, 9.40 mol, 1.00 equiv), DCM (7.0 L), H₂O (67.69 g, 3.76 mol, 0.4 equiv). This was followed by the addition of POCl₃ (474.71 g, 3.10 mol, 0.33 equiv) dropwise with stirring at 25 to 30° C. The resulting solution was stirred for 0.5 hr at 25° C. This crude solvent straight used for next step. GC-MS: (ES, m/z): M: 112

Synthesis of 3,3-dimethylcyclopentanone: Into a 20-L 4-necked round-bottom flask, was placed previous step solution 3, 3-dimethylcyclopentan-1-one in DCM (7.0 L). This was followed by the addition of DMF (1372.4 g, 2.0 equiv) dropwise with stirring at 25° C. To this was added POCl₃ (3020.22 g, 2.1 equiv) dropwise with stirring at 40° C. The resulting solution was stirred for overnight at 40° C. in an oil bath. Then the reaction mixture was quenched by the addition of 4000 g of K₃PO₄ in 30 L Water. The reaction mixture was extracted with 3×20 L of dichloromethane dried over anhydrous sodium sulfate and concentrated. This resulted in 1700 g (Crude) of 2-chloro-4, 4-dimethylcyclopent-1-ene-1-carbaldehyde as a brown solid. GC-MS: (ES, m/z): M: 158.

Synthesis of 4,4-dimethyl-1,10-diazatricyclo[6.4.0.0\hat{ }[2,6]]dodeca-2(6),7-dien-9-one: Into a 10-L 4-necked round-bottom flask, was placed 2-chloro-4,4-dimethylcyclopent-1-ene-1-carbaldehyde (1700.00 g, 10.759mol, 1.00 equiv), DMF (6 L), piperazin-2-one (1075.95 g, 10.759mol, 1.00equiv), DIEA (1665.49 g, 12.91 mol, 1.2 equiv). The resulting solution was stirred for overnight at 115° C. in an oil bath. The reaction mixture was cooled to room temperature with a water/ice bath. The solids were collected by filtration. The resulting mixture was washed with 3×6 L of H₂O and 3×4 L of PE. The solid was dried in an oven under reduced pressure. This resulted in 720 g (32.81%) of 4,4-dimethyl-1,10-diazatricyclo[6.4.0.0\hat{ }[2,6]]dodeca-2(6),7-dien-9-one as a grey solid. LC-MS: (ES, m/z): M+1: 205

Synthesis of 2-(3-bromo-5-fluoro-2-methylphenyl)-7,7-dimethyl-3,4,7,8-tetrahydro-2H-cyclopenta[4,5]pyrrolo[1,2-a]pyrazin-1(6H)-one: Into a 250 mL 3-necked round-bottom flask with DMA (100.00 mL) were added 1,3-dibromo-5-fluoro-2-methylbenzene (5.00 g, 18.662 mmol, 1.00equiv) and 7,7-dimethyl-3,4,7,8-tetrahydro-2H-cyclopenta[4,5]pyrrolo[1,2-a]pyrazin-1(6H)-one (4.57 g, 22.394 mmol, 1.20 equiv) at room temperature. To the above mixture was added CuI (2.84 g, 14.930 mmol, 0.80 equiv), K₂CO₃(7.74g, 55.986mmol, 3.00equiv) and 1,10-phenanthroline (2.02 g, 11.197 mmol, 0.60 equiv) at room temperature. The resulting mixture was stirred for 16 h at 100° C. The resulting mixture was diluted with water (200 mL). The resulting mixture was extracted with EtOAc (2×200 mL). The combined organic layers were washed with brine (1×200 mL), dried over anhydrous Na₂SO₄. After filtration, the filtrate was concentrated under reduced pressure. The residue was purified by silica gel column chromatography, eluted with CH₂Cl₂/EtOAc (1:1) to afford 2-(3-bromo-5-fluoro-2-methylphenyl)-7,7-dimethyl-3,4,7,8-tetrahydro-2H-cyclopenta[4,5]pyrrolo[1,2-a]pyrazin-1(6H)-one (1.2 g,16.43%) as a white solid. LC-MS: (ES, m/z): M+1: 391.

Synthesis of 2-(5-fluoro-2-methyl-3-(4,4,5,5-tetramethyl-1,3,2-dioxaborolan-2-yl)phenyl)-7,7-dimethyl-3,4,7,8-tetrahydro-2H-cyclopenta[4,5]pyrrolo[1,2-a]pyrazin-1(6H)-one: Into a 100 mL 3-necked round-bottom flask purged and maintained with an inert atmosphere of nitrogen, was placed 2-(3-bromo-5-fluoro-2-methylphenyl)-7,7-dimethyl-3,4,7,8-tetrahydro-2H-cyclopenta[4,5]pyrrolo[1,2-a]pyrazin-1(6H)-one (1.20 g, 3.067 mmol, 1.00 equiv), bis(pinacolato)diboron (1.56 g, 6.134 mmol, 2.00 equiv), KOAc (902.96 mg, 9.201 mmol, 3.00 equiv), Pd(dppf)Cl₂ (249.83 mg, 0.307 mmol, 0.10 equiv), dioxane (20.00mL, 236.082 mmol, 76.98 equiv). The resulting solution was stirred for 16 h at 100° C. in an oil bath. The reaction was then quenched by the addition of 30 mL of water. The resulting solution was extracted with 3×50 mL of ethyl acetate concentrated. The residue was applied onto a silica gel column and eluted with ethyl acetate/petroleum ether (1:3). The collected fractions were combined and concentrated. This resulted in 2-(5-fluoro-2-methyl-3-(4,4,5,5-tetramethyl-1,3,2-dioxaborolan-2-yl)phenyl)-7,7-dimethyl-3,4,7,8-tetrahydro-2H-cyclopenta[4,5]pyrrolo[1,2-a]pyrazin-1(6H)-one (210 mg, 15.62%) as a white solid. LC-MS: (ES, m/z): M+1: 439.

Synthesis of 5-methoxy-6-methylpyrimidin-4-amine: Into a 1-L pressure tank reactor, was placed 4, 6-dichloro-5-methoxypyrimidine (20.00 g, 111.732 mmol, 1.00 equiv), NH3.H₂O (500.00 mL). The resulting solution was stirred for overnight at 100° C. The resulting solution was cooled to R,T and diluted with 500 mL of water. The resulting solution was extracted with 3×200 mL of ethyl acetate concentrated. The residue was applied onto a silica gel column and eluted with ethyl acetate/petroleum ether(1:1). The collected fractions were combined and concentrated. This resulted in 7 g (45.02%) of 5-methoxy-6-methylpyrimidin-4-amine as a white solid. LC-MS: (ES, m/z): M+1: 160

Synthesis of 4-amino-6-chloropyrimidin-5-ol: Into a 250-mL 3-necked round-bottom flask, was placed 6-chloro-5-methoxypyrimidin-4-amine (6.50 g, 40.734 mmol, 1.00 equiv), DCM (20.00 mL). This was followed by the addition of tribromoborane (81.70 mL, mmol, 0.01 equiv) dropwise with stirring at 0° C. The resulting solution was stirred for 4 h at room temperature. The reaction mixture was quenched by slowly adding MeOH (30 mL) at −10° C., stirred at RT for 12 h, then concentrated under reduced pressure. The residue was stirred in water (30 mL) and EtOAc (30 mL). The mixture was basified with NaHCO₃ (4 mol/L) to PH 8-9. The aqueous layer was washed with EtOAc (discarded), acidified to pH with aqueous HCl solution (5 M), then extracted with EtOAc (1×50 mL) and DCM (1×50 mL). The combined organic layers were washed with brine, dried over MgSO₄, filtered, and concentrated to afford 2.7 g (45.54%) of 4-amino-6-chloropyrimidin-5-ol as a white solid. LC-MS: (ES, m/z): M+1: 146.

Synthesis of tert-butyl (S)-2-(((4-amino-6-chloropyrimidin-5-yl)oxy)methyl)pyrrolidine-1-carboxylate: Into a 250-mL 3-necked round-bottom flask purged and maintained with an inert atmosphere of nitrogen, was placed 4-amino-6-chloropyrimidin-5-ol (2.50 g, 17.176 mmol, 1.00 equiv), tert-butyl (2S)-2-(hydroxymethyl)pyrrolidine-1-carboxylate (9.68 g, 48.093 mmol, 2.80 equiv), THF (110.00 mL). This was followed by the addition of PPh₃ (11.26 g, 42.940 mmol, 2.50 equiv) at 0° C. To this was added DIAD (8.68 g, 42.940 mmol, 2.50 equiv) dropwise with stirring at 0° C. The resulting solution was stirred for 2 h at room temperature. The reaction was then quenched by the addition of 100 mL of water. The resulting solution was extracted with 3×200 mL of ethyl acetate concentrated. The residue was applied onto a silica gel column with ethyl acetate/petroleum ether (1:1). The collected fractions were combined and concentrated. This resulted in tert-butyl (S)-2-(((4-amino-6-chloropyrimidin-5-yl)oxy)methyl)pyrrolidine-1-carboxylate (175 mg, 3.10%) as a white solid. LC-MS: M+1: 329.

Synthesis of tert-butyl (S)-2-(((4-(3-(4-cyclopropyl-2-fluorobenzamido)-5-fluoro-2-methylphenyl)-6-(methylamino)pyrimidin-5-yl)oxy)methyl)pyrro lidine-1-carboxylate: Into a 50-mL round-bottom flask purged and maintained with an inert atmosphere of nitrogen, was placed tert-butyl (S)-2-(((4-amino-6-chloropyrimidin-5-yl)oxy)methyl)pyrrolidine-1-carboxylate(150.00 mg, 0.456 mmol, 1.00 equiv), 2-(5-fluoro-2-methyl-3-(4,4,5,5-tetramethyl-1,3,2-dioxaborolan-2-yl)phenyl)-7,7-dimethyl-3,4,7,8-tetrahydro-2H-cyclopenta[4,5]pyrrolo[1,2-a]pyrazin-1(6H)-one(199.98 mg, 0.456 mmol, 1 equiv), K₂CO₃(189.15 mg, 1.369 mmol, 3equiv), Pd(PPh₃)₂Cl₂(32.02 mg, 0.046 mmol, DME(5.00 mL), H₂O(0.50 mL). The resulting solution was stirred for 2 hr at 110° C. in an oil bath. The resulting solution was diluted with 15 mL of H₂O. The resulting solution was extracted with 3×20 mL of ethyl acetate concentrated. The residue was applied onto a silica gel column and eluted with ethyl acetate/petroleum ether (1:1). The collected fractions were combined and concentrated. This resulted in tert-butyl (S)-2-(((4-(3-(4-cyclopropyl-2-fluorobenzamido)-5-fluoro-2-methylphenyl)-6-(methylamino)pyrimidin-5-yl)oxy)methyl)pyrro lidine-1-carboxylate (100 mg, 36.25%) as a white solid. LC-MS: (ES, m/z): M+1: 605.

Synthesis of tert-butyl (S)-2-(((4-amino-6-(3-(7,7-dimethyl-1-oxo-1,3,4,6,7,8-hexahydro-2H-cyclopenta[4,5]pyrrolo[1,2-a]pyrazin-2-yl)-5-fluoro-2-methylphenyl)pyrimidin-5-yl(oxy)methyl)pyrrolidine-1-carboxylate: Into a 50-mL round-bottom flask, was placed tert-butyl (S)-2-(((4-(3-(4-cyclopropyl-2-fluorobenzamido)-lidine-1-carboxylate (100.00 mg, 0.165 mmol, 1.00 equiv), HCl (gas)in 1,4-dioxane (4 N, 5.00 mL).

The resulting solution was stirred for 2 h at room temperature. The resulting mixture was concentrated. This resulted in 80 mg (crude) of tert-butyl (S)-2-(((4-amino-6-(3-(7,7-dimethyl-1-oxo-1,3,4,6,7,8-hexahydro-2H-cyclopenta[4,5]pyrrolo[1,2-a]pyrazin-2-yl)-5-fluoro-2-methylphenyl)pyrimidin-5-yl)oxy)methyl)pyrrolidine-1-carboxylate e as a white solid. LC-MS: (ES, m/z): M-HCl+1: 505.

Synthesis of (S)-2-(3-(5-((1-acryloylpyrrolidin-2-yl)methoxy)-6-aminopyrimidin-4-yl)-5-fluoro-2-methylphenyl)-7,7-dimethyl-3,4,7,8-tetrahydro-2H-cyclopenta[4,5]pyrrolo[1,2-a]pyrazin-1(6H)-one: Into a 8-mL vial, was placed tert-butyl (S)-2-(((4-amino-6-(3-(7,7-dimethyl-1-oxo-1,3,4,6,7,8-hexahydro-2H-cyclopenta[4,5]pyrrolo[1,2-a]pyrazin-2-yl)-5-fluoro-2-methylphenyl)pyrimidin-5-yl)oxy)methyl)pyrrolidine-1-carboxylate (70.00 mg, 0.139 mmol, 1.00 equiv), DIEA (71.71 mg, 0.556 mmol, 4.00 equiv), DCM(1.50 mL). This was followed by the addition of acryloyl chloride (8.79 mg, 0.097 mmol, 0.70 equiv) dropwise with stirring at 0° C. The resulting solution was stirred for 2 hr at room temperature. The resulting mixture was concentrated. The crude product was purified by Prep-HPLC with the following conditions (2#SHIMADZU (HPLC-01)): Column, Halo C18, 50*3.0 mm, 2.7 μm; mobile phase, Water and ACN); Detector, 254 nm. This resulted in (S)-2-(3-(5-((1-acryloylpyrrolidin-2-yl)methoxy)-6-aminopyrimidin-4-yl)-5-fluoro-2-methylphenyl)-7,7-dimethyl-3,4,7,8-tetrahydro-2H-cyclopenta[4,5]pyrrolo[1,2-a]pyrazin-1(6H)-one (40 mg, 51.61%) as a white solid. LC-MS: (ES, m/z): M+1: 559. 1 H-NMR (300 MHz, Chloroform-d) δ 8.44 (d, J=8.7 Hz, 1H), 7.16 (dt, J=14.7, 8.6 Hz, 2H), 6.79 (d, J=10.0 Hz, 1H), 6.59-6.36 (m, 2H), 5.87-5.74 (m, 1H), 4.53 (s, 2H), 3.90 (d, J=16.7 Hz, 1H), 3.77 (t, J=9.0 Hz, 2H), 3.62 (d, J=7.7 Hz, 2H), 3.48 (t, J=8.2 Hz, 1H), 2.58 (s, 2H), 2.52 (s, 2H), 2.17 (s, 1H), 2.06 (s, 2H), 1.89 (s, 3H), 1.29 (s, 6H).

### EXAMPLE 3

### Preparation of 10-[3-(6-amino-5-[2-[(2S)-1-(prop-2-enoyl)pyrrolidin-2-yl]ethenyl]pyrimidin-4-yl)-2-methylphenyl]-4,4-dimethyl-1,10-diazatricyclo[6.4.0.0\hat{ }[2,6]]dodeca-2(6),7-dien-9-one

Synthesis of [(3,3-dimethylcyclopent-1-en-1-yl)oxy]trimethylsilane: Into a 10 L 4-necked round-bottom flask purged and maintained with an inert atmosphere of nitrogen, was placed CuCl (20.6 g, 208.0 mmol, 0.05 eq.), LiCl (17.6 g, 416.1 mmol, 0.1 eq.), tetrahydrofuran (2.5 L). This was followed by the addition of 2-cyclopenten-1-one, 3-methyl-(400.0 g, 4161.0 mmol, 1.0 eq.) at −5 to 5° C. To this was added TMSCl (474.7 g, 4369.1 mmol, 1.1 eq.) dropwise with stirring at −5 to 5° C. To the mixture was added MeMgCl (1670.0 mL, 14495.1 mmol, 3.5 eq.) dropwise with stirring at −5 to 10° C. The resulting solution was stirred for 2 hours at −5 to 10° C. in an ice/salt bath. The reaction was then quenched by the addition of MeOH (34 mL). The resulting solution was diluted with NH₄Cl (5 L). The solids were filtered out. The resulting solution was extracted with petroleum ether (3×5 L) and combined organic was dried over anhydrous sodium sulfate. After filtration, the filtrate was concentrated under reduced pressure. This resulted (780.2 g, crude) of [(3,3-dimethylcyclopent-1-en-1-yl)oxy]trimethylsilane as yellow oil. GC-MS (ES, m/z) M: 184.

Synthesis of 3,3-dimethylcyclopentanone: Into a 20 L 4-necked round-bottom flask, was placed [(3,3-dimethylcyclopent-1-en-1-yl)oxy]trimethylsilane (780.0 g, 4231.0 mmol, 1.0 eq.), CH₂Cl₂ (7.8 L), H₂O (30.5 g, 1692.4 mmol, 0.4 eq.). This was followed by the addition of POCl₃ (214.1 g, 1396.3 mmol, 0.3 eq.) dropwise with stirring at 25 to 30° C. The resulting solution was stirred for 0.5 hour at 25° C. This crude in solvent straight used for next step.GC-MS (ES, m/z) M: 112.

Synthesis of 3,3-dimethylcyclopentanone: Into a 20 L 4-necked round-bottom flask, was placed previous step solution 3,3-dimethylcyclopentan 1-one in CH₂Cl₂ (7.8 L). This was followed by the addition of N,N-dimethylformamide (619.0 g, 8.5 mol, 2.0 eq.) dropwise with stirring at 25° C. To this was added POCl₃ (1362.0 g, 17.8 mol, 2.1 eq.) dropwise with stirring at 40° C. The resulting solution was stirred for overnight at 40° C. The reaction mixture was cooled to 25° C. The reaction was then quenched by the addition of K₃PO₄ (2000 g) in H₂O (5 L). The resulting solution was extracted with dichloromethane (3×10 L) and combined organic was dried over anhydrous sodium sulfate. After filtration, the filtrate was concentrated under reduced pressure. This resulted in This resulted (530 g, Crude) of 2-chloro-4,4-dimethylcyclopent-1-ene-1-carbaldehyde as a brown solid. GC-MS (ES, m/z): 158.

Synthesis of 4,4-dimethyl-1,10-diazatricyclo[6.4.0.0\hat{ }[2,6]]dodeca-2(6),7-dien-9-one: Into a 5 L 4-necked round-bottom flask, was placed 2-chloro-4,4-dimethylcyclopent-1-ene-1-carbaldehyde (474.0 g, 2988.1 mmol, 1.0 eq.), N,N-dimethylformamide (3 L), piperazin-2-one (299.2 g, 2988.1 mmol, 1.0 eq.), DIEA (463.4 g, 3585.7 mmol, 1.2 eq.). The resulting solution was stirred for overnight at 115° C. in an oil bath. The reaction mixture was cooled to 25° C. The solids were collected by filtration. The resulting mixture was washed with H₂O (3>2 L) and petroleum ether (3×2 L). The combined organic was dried over anhydrous sodium sulfate. After filtration, the filtrate was concentrated under reduced pressure. This resulted (30.0 g, 37.7%) of 4,4-dimethyl-1,10-diazatricyclo[6.4.0.0\hat{ }[2,6]]dodeca-2(6),7-dien-9-one as grey solid. LC-MS (ES, m/z) M+1: 205.

Synthesis of 10-(3-bromo-2-methylphenyl)-4,4-dimethyl-1,10-diazatricyclo[6.4.0.0\hat{ }[2,6]]dodeca-2(6),7-dien-9-one: Into a 1000 mL round-bottom flask purged and maintained with an inert atmosphere of nitrogen, was placed 1,3-dibromo-2-methylbenzene (30.0 g\hat{ }121.0 mmol, 2.0 eq.), 4,4-dimethyl-1,10-diazatricyclo[6.4.0.0\hat{ }[2,6]]dodeca-2(6),7-dien-9-one (12.3 g, 60.5 mmol, 1.00 eq.), K₂CO₃ (59.0 g, 181.5 mmol, 3.0 eq.), CuI (6.9 g, 36.3 mmol, 0.6 eq.), N,N-dimethylformamide (350 mL), 1,10-phenanthroline (6.5 g, 36.3 mmol, 0.6 eq.). The resulting solution was stirred for 24 hours at 120° C. in an oil bath. The reaction mixture was cooled to 25° C. with a water/ice bath. The reaction was then quenched by the addition of 500 mL of H₂O. The solids were filtered out. The resulting solution was extracted with 2×200 mL of ethyl acetate and the organic layers combined. The resulting mixture was washed with 5×300 mL of H₂O and 2×400 mL of brine. The mixture was dried over anhydrous Na₂SO₄. After filtration, the filtrate was concentrated under reduced pressure. The residue was purified by a flash column (silica gel, ethyl acetate/petroleum ether=1:1). This resulted in 7.5g (crude) of 10-(3-bromo-2-methylphenyl)-4,4-dimethyl-1,10-diazatricyclo[6.4.0.0\hat{ }[2,6]]dodeca-2(6),7-dien-9-one as ayellow solid. LC-MS (ESI, m/z) M+1: 373. 1 HNMR (300 MHz, Chloroform-d) δ 7.54-7.56 (d, J=6.0 Hz, 1H), 7.10-7.21 (m, 2H), 6.81 (s, 1H), 4.03-4.24 (m, 3H), 3.76-3.83 (m, 1H), 2.51-2.56 (m, 4H), 2.37 (s, 3H), 1.24-1.28 (m, 6H).

Synthesis of 4,4-dimethyl-10- [2-methyl-3-(4,4,5,5-tetramethyl-1,3,2-dioxaborolan-2-yl)phenyl]-1,10-diazatricyclo[6.4.0.0\hat{ }[2,6] ]dodeca-2(6),7-dien-9-one: Into a 500 mL round-bottom flask purged and maintained with an inert atmosphere of nitrogen, was placed 10-(3-bromo-2-methylphenyl)-4,4-dimethyl-1,10-diazatricyclo[6.4.0.0\hat{ }[2,6]]dodeca-2(6),7-dien-9-one (7.4 g, 19.9 mmol, 1.0 eq.), bis(pinacolato)diboron (6.0 g, 23.9 mmol, 1.2 eq.), KOAc (6.4 g, 49.4 mmol, 2.5 eq.), Pd(dppf)Cl₂ (1.4 g, 2.0 mmol, 0.1 eq.), toluene (200 mL). The resulting solution was stirred for 2 hours at 100° C. in an oil bath. The reaction mixture was cooled to 25° C. The resulting mixture was concentrated. The resulting solution was diluted with 300 mL of ethyl acetate. The solids were filtered out. The mixture was dried over anhydrous sodium sulfate. After filtration, the filtrate was concentrated under reduced pressure. The residue was purified by a flash column (silica gel, ethyl acetate/petroleum ether=1:1). This resulted in 8.8 g of 4,4-dimethyl-10-[2-methyl-3-(4,4,5,5-tetramethyl-1,3,2-dioxaborolan-2-yl)phenyl]-1,10-diazatricyclo[6.4.0.0\hat{ }[2,6]]dodeca-2(6),7-dien-9-one as yellow solid. LC-MS (ESI, m/z) M+1: 421.

Synthesis of 2-(dichloromethyl)-4,4,5,5-tetramethyl-1,3,2-dioxaborolane: Into a 1 L round-bottom flask, was placed methylene chloride (46.2 g, 543.9 mmol, 1.0 eq.), THF (500 mL). This was followed by the addition of 2.5 M n-BuLi (217 mL, 543.9 mmol, 1.0 eq.) dropwise with stirring at −100° C. in 30 min. To this was added trimethyl borate (56.5 g, 543.9 mmol, 1.0 eq.) at −100° C., the resulting solution was stirred for 1 hour at −100° C. The reaction was then quenched by the addition of 100 mL of HCl (5M). The resulting solution was extracted with 3×200 mL of Et₂O dried over anhydrous magnesium sulfate. After filtration, the filtrate was concentrated under reduced pressure. this was followed by addition of benzene (500 mL), pinacol (64.3 g, 544.0 mmol, 1.0 eq.). The resulting solution was stirred while the temperature was maintained at 90° C. in an oil bath. The reaction mixture was cooled to 25° C. The crude product was purified by distillation under reduced pressure (0.1 MPa) and the fraction was collected at 130° C. This resulted in 48 g (41.8%) of 2-(dichloromethyl)-4,4,5,5-tetramethyl-1,3,2-dioxaborolane as white solid. 1 HNMR (300 MHz, DMSO-d₆) δ 5.34 (s, 1H), 1.12 (s, 12H).

Synthesis of tert-butyl (2S)-2-formylpyrrolidine-1-carboxylate: Into a 250 mL round-bottom flask, was placed tert-butyl (2S)-2-(hydroxymethyl)pyrrolidine-1-carboxylate (8.0 g, 39.7mmol, 1.0 eq.), CH₂Cl₂(100 mL), DMP (30.3 g, 71.5 mmol, 1.8 eq.). The resulting solution was stirred for overnight at 25° C. The solids were filtered out. The resulting solution was diluted with 100 mL of H₂O. The resulting solution was extracted with 3×100 mL of dichloromethane dried over anhydrous sodium sulfate. After filtration, the filtrate was concentrated under reduced pressure. The crude residue was purified by a flash column (silica gel, ethyl acetate/petroleum ether=1:3). The collected fractions were combined and concentrated. This resulted in 4.5 g (56.8%) of tert-butyl (2S)-2-formylpyrrolidine-1-carboxylate as light yellow oil. LC-MS (ESI, m/z) M+1: 200.

Synthesis of tert-butyl (2S)-2-[2-(4,4,5,5-tetramethyl-1,3,2-dioxaborolan-2-ypethenyl]pyrrolidine-1-carboxylate: Into a 250 mL 3-necked round-bottom flask purged and maintained with an inert atmosphere of nitrogen, was placed CrCl₃ (15.9 g, 129.7 mmol, 8.0 eq.), tetrahydrofuran (130 mL), tert-butyl (2S)-2-formylpyrrolidine-1-carboxylate (3.2 g, 16.2mmol, 1.0 eq.), 2-(dichloromethyl)-4,4,5,5-tetramethyl-1,3,2-dioxaborolane (6.8 g, 32.4 mmol, 2.0 eq.). This was followed by the addition of LiI (8.7 g, 64.8 mmol, 4.0 eq.) in tetrahydrofuran (10 mL) dropwise with stirring at 25° C. The resulting solution was stirred for overnight at 25° C. The reaction was then quenched by the addition of 100 mL of H₂O. The resulting solution was extracted with 3×100 mL of CH₂Cl₂. The combined organic layers were washed with brine (1×200 mL), dried over anhydrous Na₂SO₄. After filtration, the filtrate was concentrated under reduced pressure. The crude residue was purified by a flash column (silica gel, ethyl acetate/petroleum ether=1:10). The collected fractions were combined and concentrated. This resulted in 1.9 g (36.2%) of tert-butyl (2S)-2-[2-(4,4,5,5-tetramethyl-1,3,2-dioxaborolan-2-yl)ethenyl]pyrrolidine-1-carboxylate as yellow oil. LC-MS (ESI, m/z) M+1: 324.

Synthesis of tert-butyl (2S)-2-[2-(4-amino-6-chloropyrimidin-5-ypethenylipyrrolidine-1-carboxylate: Into a 100 mL round-bottom flask purged and maintained with an inert atmosphere of nitrogen, was placed tert-butyl (2S)-242-(4,4,5,5-tetramethyl-1,3,2-dioxaborolan-2-yl)ethenyl]pyrrolidine-1-carboxylate (2.4 g, 7.5 mmol, 1.0 eq.), DME (50 mL), H₂O (5 mL), 6-chloro-5-iodopyrimidin-4-amine (1.9 g, 7.5 mmol, 1.0 eq.), Na₂CO₃ (2.4 g, 22.5 mmol, 3.0 eq.), Pd(PPh)₃Cl₂ (527.6 mg, 0.7 mmol, 0.1 eq.). The resulting solution was stirred for overnight at 80° C. in an oil bath. The reaction mixture was cooled to 25° C. The reaction was then quenched by the addition of 50 mL of H₂O. The resulting solution was extracted with 3×70 mL of ethyl acetate. The combined organic phase was dried over anhydrous magnesium sulfate. After filtration, the filtrate was concentrated under reduced pressure. The crude residue was purified by a flash column (silica gel, ethyl acetate/petroleum ether=1:1). The collected fractions were combined and concentrated. This resulted in 1.2 g (49.1%) of tert-butyl (2S)-2-[2-(4-amino-6-chloropyrimidin-5-yl)ethenyl]pyrrolidine-1-carboxylate as white solid. LC-MS (ESI, m/z) M+1: 325.

Synthesis of tert-butyl (2S)-2-[2-[4-amino-6-(3-[4,4-dimethyl-9-oxo-1,10-diazatricyclo[6.4.0.0\hat{ }[2,6]]dodeca-2(6),7-dien-10-yl]-2-methylphenyl)pyrimidin-5-yl]ethenyl]pyrrolidine-1-carboxylate Into a 20 mL vial purged and maintained with an inert atmosphere of nitrogen, was placed 4,4-dimethyl-10-[2-methyl-3-(4,4,5,5-tetramethyl-1,3,2-dioxaborolan-2-yl)phenyl]-1,10-diazatricyclo[6.4.0.0\hat{ }[2,6]]dodeca-2(6),7-dien-9-one (400.0 mg, 0.9 mmol, 1.2 eq.), tert-butyl (2S)-2-[2-(4-amino-6-chloropyrimidin-5-yl)ethenyl]pyrrolidine-1-carboxylate (257.5 mg, 0.8 mmol, 1.0 eq.), K₂CO₃ (219.1 mg, 1.6 mmol, 2.0 eq.), Pd(PPh₃)₂Cl₂ (72.0 mg, 0.1 mmol, 0.1 eq.), DME (4 mL), H₂O (0.4 mL). The resulting solution was stirred for 3 hours at 100° C. The reaction mixture was cooled to 25° C. The resulting solution was diluted with 10 mL of ethyl acetate and 10 mL of H₂O. The resulting solution was extracted with 3×10 mL of ethyl acetate and the organic layers combined and dried over anhydrous sodium sulfate. After filtration, the filtrate was concentrated under reduced pressure. The crude residue was purified by a flash column (silica gel, ethyl acetate/petroleum ether=0:100-2:1). This resulted in 300 mg (64.9%) of tert-butyl (2S)-2-[2-[4-amino-6-(3-[4,4-dimethyl-9-oxo-1,10-diazatricyclo[6.4.0.0\hat{ }[2,6]]dodeca-2(6),7-dien-10-yl]-2-methylphenyl)pyrimidin-5-yl]ethenyl]pyrrolidine- 1 -carboxylate as colorless oil. LC-MS (ESI, m/z) M+1:583. 1 HNMR (300 MHz, DMSO-d₆) δ 8.32 (s, 1H), 7.29 (q, J=5.9 Hz, 2H), 7.05 (s, 1H), 6.70 (s, 2H), 6.48 (d, J=1.6 Hz, 1H), 5.97 (s, 1H), 5.49 (d, J=17.2 Hz, 1H), 4.14 (s, 4H), 3.71 (s, 1H), 3.18 (d, J=5.2 Hz, 1H), 3.14 (s, 2H), 2.56 (s, 2H), 2.41 (s, 2H), 1.89 (s, 3H), 1.82 (s, 1H), 1.62 (s, 2H), 1.40-1.31 (m, 9H), 1.25-1.13 (m, 6H).

Synthesis of 10-[3-(6-amino-5-[2-[(2S)-pyrrolidin-2-yl]ethenyl]pyrimidin-4-yl)-2-methylphenyl]-4,4-dimethyl-1,10-diazatricyclo[6.4.0.0\hat{ }[2,6] ]dodeca-2(6),7-dien-9-one hydrochloride: Into a 100 mL round-bottom flask, was placed tert-butyl (2S)-2-[2-[4-amino-6-(3-[4,4-dimethyl-9-oxo-1,10-diazatricyclo[6.4.0.0\hat{ }[2,6]]dodeca-2(6),7-dien-10-yl]-2-methylphenyl)pyrimidin-5-yl]ethenyl]pyrrolidine-1-carboxylate (300.0 mg, 0.5 mmol, 1.0 eq.). This was followed by the addition of 4 M HCl (gas)in 1,4-dioxane (3 mL) at 25° C. The resulting solution was stirred for 2 hours at 25° C. The resulting mixture was concentrated. This resulted in 200 mg (74.8%) of 10-[3-(6-amino-5-[2-[(2S)-pyrrolidin-2-yl]ethenyl]pyrimidin-4-yl)-2-methylphenyl]-4,4-dimethyl-1,10-diazatricyclo[6.4.0.0\hat{ }[2,6]]dodeca-2(6),7-dien-9-one hydrochloride as off-white solid. LC-MS (ESI, m/z) M+1: 483.

Synthesis of 10-[3-(6-amino-5-[2-[(2S)-1-(prop-2-enoyl)pyrrolidin-2-yl]ethenyl]pyrimidin-4-yl)-2-methylphenyl]-4,4-dimethyl-1,10-diazatricyclo[6.4.0.0\hat{ }[2,6]]dodeca-2(6),7-dien-9-one: Into a 8 mL vial, was placed 10-[3-(6-amino-5-[2-[(2S)-pyrrolidin-2-yl]ethenyl]pyrimidin-4-yl)-2-methylphenyl]-4,4-dimethyl-1,10-diazatricyclo[6.4.0.0\hat{ }[2,6]]dodeca-2(6),7-dien-9-one hydrochloride (60.0 mg, 0.1 mmol, 1.0 eq.), CH₂Cl₂ (1 mL), trimethylamine (23.4 mg, 0.2 mmol, 2.0 eq.). This was followed by the addition of a solution of acryloyl chloride (10.4 mg, 0.1 mmol, 1.0 eq.) in CH₂Cl₂ (0.2 mL) dropwise with stirring at 25° C. The resulting solution was stirred for 2 hours at 25° C. The resulting mixture was concentrated. The crude product was purified by Prep-HPLC with the following conditions: Column, X Bridge Shield RP18 OBD Column, 5 um, 19*150 mm; mobile phase, H₂O (0.05% TFA) and CH₃CN (26% Phase B up to 50% in 7 min); Detector, UV 254/220 nm. The collected solution was dried by lyophilization. This resulted in 31 mg (49.9%) of 10-[3-(6-amino-5-[2-[(2S)-1-(prop-2-enoyl)pyrrolidin-2-yl]ethenyl]pyrimidin-4-yl)-2-methylphenyl]-4,4-dimethyl-1,10-diazatricyclo[6.4.0.0\hat{ }[2,6]]dodeca-2(6),7-dien-9-one as white solid.LC-MS (ESI, m/z) M+1: 537. ¹HNMR (300 MHz, DMSO-d₆) δ 8.30 (d, J=2.5 Hz, 1H), 7.33-7.20 (m, 2H), 7.01 (s, 1H), 6.806.68 (m, 2H),6.57-6.42 (m, 1H), 6.43-6.30 (m, 1H), 6.12-5.99 (m, 1H), 5.93 (d,

J=16.1 Hz, 1H), 5.69-5.38 (m, 2H), 4.43 (d, J=27.8 Hz, 2H), 4.23-3.98 (m, 3H), 3.70 (s, 1H), 3.46-3.14 (m, 2H), 2.55 (s, 2H), 2.41 (s, 2H), 1.86 (d, J=16.2 Hz, 3H), 1.64 (s, 1H), 1.55 (s, 1H), 1.44 (s, 1H), 1.21 (s, 6H).

### EXAMPLE 4

### Preparation of 10-[3-(6-amino-5-[2-[(2S)-1-(prop-2-enoyl)pyrrolidin-2-yl]ethenyl]pyrimidin-4-yl)-2-methylphenyl]-4,4-dimethyl-1,10-diazatricyclo[6.4.0.0\hat{ }[2,6]]dodeca-2(6),7-dien-9-one (as a negative control)

Synthesis of [(3,3-dimethylcyclopent-1-en-1-ypoxy]trimethylsilane: Into a 10 L 4-necked round-bottom flask purged and maintained with an inert atmosphere of nitrogen, was placed CuCl (20.6 g, 208.0 mmol, 0.05 eq.), LiCl (17.6 g, 416.1 mmol, 0.1 eq.), tetrahydrofuran (2.5 L). This was followed by the addition of 2-cyclopenten-1-one, 3-methyl-(400.0 g, 4161.0 mmol, 1.0 eq.) at −5 to 5° C. To this was added TMSCl (474.7 g, 4369.1 mmol, 1.1 eq.) dropwise with stirring at −5 to 5° C. To the mixture was added MeMgCl (1670.0 mL, 14495.1 mmol, 3.5 eq.) dropwise with stirring at −5 to 10° C. The resulting solution was stirred for 2 hours at −5 to 10° C. in an ice/salt bath. The reaction was then quenched by the addition of MeOH (34 mL). The resulting solution was diluted with NH₄Cl (5 L). The solids were filtered out. The resulting solution was extracted with petroleum ether (3×5 L) and combined organic was dried over anhydrous sodium sulfate. After filtration, the filtrate was concentrated under reduced pressure. This resulted (780.2 g, crude) of [(3,3-dimethylcyclopent-1-en-1-yl)oxy]trimethylsilane as yellow oil. GC-MS (ES, m/z) M: 184.

Synthesis of 3,3-dimethylcyclopentanone: Into a 20 L 4-necked round-bottom flask, was placed [(3,3-dimethylcyclopent-1-en-1-yl)oxy]trimethylsilane (780.0 g, 4231.0 mmol, 1.0 eq.), CH₂Cl₂ (7.8 L), H₂O (30.5 g, 1692.4 mmol, 0.4 eq.). This was followed by the addition of POCl₃ (214.1 g, 1396.3 mmol, 0.3 eq.) dropwise with stirring at 25 to 30° C. The resulting solution was stirred for 0.5 hour at 25° C. This crude in solvent straight used for next step.GC-MS (ES, m/z) M: 112

Synthesis of 3,3-dimethylcyclopentanone: Into a 20 L 4-necked round-bottom flask, was placed previous step solution 3,3-dimethylcyclopentan-1-one in CH₂Cl₂ (7.8 L). This was followed by the addition of N,N-dimethylformamide (619.0 g, 8.5 mol, 2.0 eq.) dropwise with stirring at 25° C. To this was added POCl₃ (1362.0 g, 17.8 mol, 2.1 eq.) dropwise with stirring at 40° C. The resulting solution was stirred for overnight at 40° C. The reaction mixture was cooled to 25° C. The reaction was then quenched by the addition of K₃PO₄ (2000 g) in H₂O (5 L). The resulting solution was extracted with dichloromethane (3×10 L) and combined organic was dried over anhydrous sodium sulfate. After filtration, the filtrate was concentrated under reduced pressure. This resulted in This resulted (530 g, Crude) of 2-chloro-4,4-dimethylcyclopent-1-ene-1-carbaldehyde as a brown solid. GC-MS (ES, m/z): 158.

Synthesis of 4,4-dimethyl-1,10-diazatricyclo[6.4.0.0\hat{ }[2,6]]dodeca-2(6),7-dien-9-one: Into a 5 L 4-necked round-bottom flask, was placed 2-chloro-4,4-dimethylcyclopent-1-ene-1-carbaldehyde (474.0 g, 2988.1 mmol, 1.0 eq.), N,N-dimethylformamide (3 L), piperazin-2-one (299.2 g, 2988.1 mmol, 1.0 eq.), DIEA (463.4 g, 3585.7 mmol, 1.2 eq.). The resulting solution was stirred for overnight at 115° C. in an oil bath. The reaction mixture was cooled to 25° C. The solids were collected by filtration. The resulting mixture was washed with H₂O (3×2 L) and petroleum ether (3×2 L). The combined organic was dried over anhydrous sodium sulfate. After filtration, the filtrate was concentrated under reduced pressure. This resulted (30.0 g, 37.7%) of 4,4-dimethyl-1,10-diazatricyclo[6.4.0.0\hat{ }[2,6]]dodeca-2(6),7-dien-9-one as grey solid. LC-MS (ES, m/z) M+1: 205.

Synthesis of 10-(3-bromo-2-methylphenyl)-4,4-dimethyl-1,10-diazatricyclo[6.4.0.0\hat{ }[2,6]]dodeca-2(6),7-dien-9-one: Into a 1000 mL round-bottom flask purged and maintained with an inert atmosphere of nitrogen, was placed 1,3-dibromo-2-methylbenzene (30.0 g, 121.0 mmol, 2.0 eq.), 4,4-dimethyl-1,10-diazatricyclo[6.4.0.0\hat{ }[2,6]]dodeca-2(6),7-dien-9-one (12.3 g, 60.5 mmol, 1.00 eq.), K₂CO₃ (59.0 g, 181.5 mmol, 3.0 eq.), CuI (6.9 g, 36.3 mmol, 0.6 eq.), N,N-dimethylformamide (350 mL), 1,10-phenanthroline (6.5 g, 36.3 mmol, 0.6 eq.). The resulting solution was stirred for 24 hours at 120° C. in an oil bath. The reaction mixture was cooled to 25° C. with a water/ice bath. The reaction was then quenched by the addition of 500 mL of H₂O. The solids were filtered out. The resulting solution was extracted with 2×200 mL of ethyl acetate and the organic layers combined. The resulting mixture was washed with 5×300 mL of H₂O and 2×400 mL of brine. The mixture was dried over anhydrous Na₂SO₄. After filtration, the filtrate was concentrated under reduced pressure. The residue was purified by a flash column (silica gel, ethyl acetate/petroleum ether=1:1). This resulted in 7.5g (crude) of 10-(3-bromo-2-methylphenyl)-4,4-dimethyl-1,10-diazatricyclo[6.4.0.0\hat{ }[2,6]]dodeca-2(6),7-dien-9-one as ayellow solid. LC-MS (ESI, m/z) M+1: 373. 1 HNMR (300 MHz, Chloroform-d) δ 7.54-7.56 (d, J=6.0 Hz, 1H), 7.10-7.21 (m, 2H), 6.81 (s, 1H), 4.03-4.24 (m, 3H), 3.76-3.83 (m, 1H), 2.51-2.56 (m, 4H), 2.37 (s, 3H), 1.24-1.28 (m, 6H).

Synthesis of 4,4-dimethyl-10-[2-methyl-3-(4,4,5,5-tetramethyl-1,3,2-dioxaborolan-2-yl)phenyl]-1,10-diazatricyclo[6.4.0.0\hat{ }[2,6]]dodeca-2(6),7-dien-9-one: Into a 500 mL round-bottom flask purged and maintained with an inert atmosphere of nitrogen, was placed 10-(3-bromo-2-methylphenyl)-4,4-dimethyl-1,10-diazatricyclo[6.4.0.0\hat{ }[2,6]]dodeca-2(6),7-dien-9-one (7.4 g, 19.9 mmol, 1.0 eq.), bis(pinacolato)diboron (6.0 g, 23.9 mmol, 1.2 eq.), KOAc (6.4 g, 49.4 mmol, 2.5 eq.), Pd(dppf)Cl₂ (1.4 g, 2.0 mmol, 0.1 eq.), toluene (200 mL). The resulting solution was stirred for 2 hours at 100° C. in an oil bath. The reaction mixture was cooled to 25° C. The resulting mixture was concentrated. The resulting solution was diluted with 300 mL of ethyl acetate. The solids were filtered out. The mixture was dried over anhydrous sodium sulfate. After filtration, the filtrate was concentrated under reduced pressure. The residue was purified by a flash column (silica gel, ethyl acetate/petroleum ether=1:1). This resulted in 8.8 g of 4,4-dimethyl-10-[2-methyl-3-(4,4,5,5-tetramethyl-1,3,2-dioxaborolan-2-yl)phenyl]-1,10-diazatricyclo[6.4.0.0\hat{ }[2,6]]dodeca-2(6),7-dien-9-one as yellow solid. LC-MS (ESI, m/z) M+1: 421.

Synthesis of 2-(dichloromethyl)-4,4,5,5-tetramethyl-1,3,2-dioxaborolane: Into a 1 L round-bottom flask, was placed methylene chloride (46.2 g, 543.9 mmol, 1.0 eq.), THF (500 mL). This was followed by the addition of 2.5 M n-BuLi (217 mL, 543.9 mmol, 1.0 eq.) dropwise with stirring at −100° C. in 30 min. To this was added trimethyl borate (56.5 g, 543.9 mmol, 1.0 eq.) at −100° C., the resulting solution was stirred for 1 hour at −100° C. The reaction was then quenched by the addition of 100 mL of HCl (5M). The resulting solution was extracted with 3×200 mL of Et₂O dried over anhydrous magnesium sulfate. After filtration, the filtrate was concentrated under reduced pressure. this was followed by addition of benzene (500 mL), pinacol (64.3 g, 544.0 mmol, 1.0 eq.). The resulting solution was stirred while the temperature was maintained at 90° C. in an oil bath. The reaction mixture was cooled to 25° C. The crude product was purified by distillation under reduced pressure (0.1 MPa) and the fraction was collected at 130° C. This resulted in 48 g (41.8%) of 2-(dichloromethyl)-4,4,5,5-tetramethyl-1,3,2-dioxaborolane as white solid. 1 HNMR (300 MHz, DMSO-d6) δ 5.34 (s, 1H), 1.12 (s, 12H).

Synthesis of tert-butyl (2S)-2-formylpyrrolidine-1-carboxylate: Into a 250 mL round-bottom flask, was placed tert-butyl (2S)-2-(hydroxymethyl)pyrrolidine-1-carboxylate (8.0 g, 39.7 mmol, 1.0 eq.), CH₂Cl₂(100 mL), DMP (30.3 g, 71.5 mmol, 1.8 eq.). The resulting solution was stirred for overnight at 25° C. The solids were filtered out. The resulting solution was diluted with 100 mL of H₂O. The resulting solution was extracted with 3×100 mL of dichloromethane dried over anhydrous sodium sulfate. After filtration, the filtrate was concentrated under reduced pressure. The crude residue was purified by a flash column (silica gel, ethyl acetate/petroleum ether=1:3). The collected fractions were combined and concentrated. This resulted in 4.5 g (56.8%) of tert-butyl (2S)-2-formylpyrrolidine-1-carboxylate as light yellow oil. LC-MS (ESI, m/z) M+1: 200.

Synthesis of tert-butyl (2S)-2-[2-(4,4,5,5-tetramethyl-1,3,2-dioxaborolan-2-ypethenyl]pyrrolidine-1-carboxylate: Into a 250 mL 3-necked round-bottom flask purged and maintained with an inert atmosphere of nitrogen, was placed CrCl₃ (15.9 g, 129.7 mmol, 8.0 eq.), tetrahydrofuran (130 mL), tert-butyl (2S)-2-formylpyrrolidine-1-carboxylate (3.2 g, 16.2mmol, 1.0 eq.), 2-(dichloromethyl)-4,4,5,5-tetramethyl-1,3,2-dioxaborolane (6.8 g, 32.4 mmol, 2.0 eq.). This was followed by the addition of LiI (8.7 g, 64.8 mmol, 4.0 eq.) in tetrahydrofuran (10 mL) dropwise with stirring at 25° C. The resulting solution was stirred for overnight at 25° C. The reaction was then quenched by the addition of 100 mL of H₂O. The resulting solution was extracted with 3×100 mL of CH₂Cl₂. The combined organic layers were washed with brine (1×200 mL), dried over anhydrous Na₂SO₄. After filtration, the filtrate was concentrated under reduced pressure. The crude residue was purified by a flash column (silica gel, ethyl acetate/petroleum ether=1:10). The collected fractions were combined and concentrated. This resulted in 1.9 g (36.2%) of tert-butyl (2S)-2-[2-(4,4,5,5-tetramethyl-1,3,2-dioxaborolan-2-yl)ethenyl]pyrrolidine-1-carboxylate as yellow oil. LC-MS (ESI, m/z) M+1: 324.

Synthesis of tert-butyl (2S)-2-[2-(4-amino-6-chloropyrimidin-5-yl)ethenyl]pyrrolidine-1-carboxylate: Into a 100 mL round-bottom flask purged and maintained with an inert atmosphere of nitrogen, was placed tert-butyl (2S)-2-[2-(4,4,5,5-tetramethyl-1,3,2-dioxaborolan-2-yl)ethenyl]pyrrolidine-1-carboxylate (2.4 g, 7.5 mmol, 1.0 eq.), DME (50 mL), H₂O (5 mL), 6-chloro-5-iodopyrimidin-4-amine (1.9 g, 7.5 mmol, 1.0 eq.), Na₂CO₃ (2.4 g, 22.5 mmol, 3.0 eq.), Pd(PPh)₃Cl₂ (527.6 mg, 0.7 mmol, 0.1 eq.). The resulting solution was stirred for overnight at 80° C. in an oil bath. The reaction mixture was cooled to 25° C. The reaction was then quenched by the addition of 50 mL of H₂O. The resulting solution was extracted with 3×70 mL of ethyl acetate. The combined organic phase was dried over anhydrous magnesium sulfate. After filtration, the filtrate was concentrated under reduced pressure. The crude residue was purified by a flash column (silica gel, ethyl acetate/petroleum ether=1:1). The collected fractions were combined and concentrated. This resulted in 1.2 g (49.1%) of tert-butyl (2S)-2-[2-(4-amino-6-chloropyrimidin-5-yl)ethenyl]pyrrolidine-1-carboxylate as white solid. LC-MS (ESI, m/z) M+1: 325.

Synthesis of tert-butyl (2S )-2-[2-[4-amino-6-(3-[4,4-dimethyl-9-oxo-1,10-diazatricyclo [6.4.0.0\hat{ }[2,6]]dodeca-2(6),7-dien- 10- yl]-2-methylphenyl)pyrimidin-5-yl]ethenyl]pyrrolidine-1-carboxylate Into a 20 mL vial purged and maintained with an inert atmosphere of nitrogen, was placed 4,4-dimethyl-10-[2-methyl-3-(4,4,5,5-tetramethyl-1,3,2-dioxaborolan-2-yl)phenyl]-1,10-diazatricyclo[6.4.0.0\hat{ }\hat{ }[2,6]]dodeca-2(6),7-dien-9-one (400.0 mg, 0.9 mmol, 1.2 eq.), tert-butyl (2S)-2-[2-(4-amino-6-chloropyrimidin-5-yl)ethenyl]pyrrolidine-1-carboxylate (257.5 mg, 0.8 mmol, 1.0 eq.), K2CO₃ (219.1 mg, 1.6 mmol, 2.0 eq.), Pd(PPh₃)₂Cl₂ (72.0 mg, 0.1 mmol, 0.1 eq.), DME (4 mL), H₂O (0.4 mL). The resulting solution was stirred for 3 hours at 100° C. The reaction mixture was cooled to 25° C. The resulting solution was diluted with 10 mL of ethyl acetate and 10 mL of H₂O. The resulting solution was extracted with 3×10 mL of ethyl acetate and the organic layers combined and dried over anhydrous sodium sulfate. After filtration, the filtrate was concentrated under reduced pressure. The crude residue was purified by a flash column (silica gel, ethyl acetate/petroleum ether=0:100-2:1). This resulted in 300 mg (64.9%) of tert-butyl (2S)-2-[2-[4-amino-6-(3-[4,4-dimethyl-9-oxo-1,10-diazatricyclo[6.4.0.0\hat{ }[2,6]]dodeca-2(6),7-dien-10-yl]-2-methylphenyl)pyrimidin-5-yl]ethenyl]pyrrolidine- 1 -carboxylate as colorless oil. LC-MS (ESI, m/z) M+1:583. 1 HNMR (300 MHz, DMSO-d₆) δ 8.32 (s, 1H), 7.29 (q, J=5.9 Hz, 2H), 7.05 (s, 1H), 6.70 (s, 2H), 6.48 (d, J=1.6 Hz, 1H), 5.97 (s, 1H), 5.49 (d, J=17.2 Hz, 1H), 4.14 (s, 4H), 3.71 (s, 1H), 3.18 (d, J=5.2 Hz, 1H), 3.14 (s, 2H), 2.56 (s, 2H), 2.41 (s, 2H), 1.89 (s, 3H), 1.82 (s, 1H), 1.62 (s, 2H), 1.40-1.31 (m, 9H), 1.25-1.13 (m, 6H).

Synthesis of 10-[3-(6-amino-5-2-[(2S)-pyrrolidin-2-yl]ethenyllpyrimidin-4-yl)-2-methylphenyl]-4,4-dimethyl-1,10-diazatricyclo[6.4.0.0\hat{ }[2,6] ]dodeca-2(6),7-dien-9-one hydrochloride: Into a 100 mL round-bottom flask, was placed tert-butyl (2S)-2-[2[4-amino-6-(3-[4,4-dimethyl-9-oxo-1,10-diazatricyclo[6.4.0.0\hat{ }[2,6]]dodeca-2(6),7-dien-10-yl]-2-methylphenyl)pyrimidin-5-yl]ethenyl]pyrrolidine-1-carboxylate (300.0 mg, 0.5 mmol, 1.0 eq.). This was followed by the addition of 4 M HCl (gas) in 1,4-dioxane (3 mL) at 25° C. The resulting solution was stirred for 2 hours at 25° C. The resulting mixture was concentrated. This resulted in 200 mg (74.8%) of 10-[3-(6-amino-5-[2-[(2S)-pyrrolidin-2-yl]ethenyl]pyrimidin-4-yl)-2-methylphenyl]-4,4-dimethyl-1,10-diazatricyclo[6.4.0.0\hat{ }[2,6]]dodeca-2(6),7-dien-9-one hydrochloride as off-white solid. LC-MS (ESI, m/z) M+1: 483.

Synthesis of 10-[3-(6-amino-5-[2-[(2S)-1-propanoylpyrrolidin-2-yl]ethenyl]pyrimidin-4-yl)-2-methylphenyl]-4,4-dimethyl-1,10-diazatricyclo[6.4.0.0\hat{ }[2,6]]dodeca-2(6),7-dien-9-one: Into a 8 mL vial, was placed 10-[3-(6-amino-5-[2-[(2S)-pyrrolidin-2-yl]ethenyl]pyrimidin-4-yl)-2-methylphenyl]-4,4-dimethyl-1,10-diazatricyclo[6.4.0.0\hat{ }[2,6]]dodeca-2(6),7-dien-9-one hydrochloride (60.0 mg, 0.1 mmol, 1.0 eq.), CH₂Cl₂ (1 mL), trimethylamine (23.4 mg, 0.2 mmol, 2.0 eq.). This was followed by the addition of a solution of propanoyl chloride (10.7 mg, 0.1 mmol, 1.0 eq.) in CH₂Cl₂ (0.2 mL) dropwise with stirring at 25° C. The resulting solution was stirred for 2 hours at 25° C. The resulting mixture was concentrated. The crude product was purified by Prep-HPLC with the following conditions: Column, X Bridge Shield RP18 OBD Column, 19*150 mm; mobile phase, H₂O (0.05% TFA) and CH₃CN (26% Phase B up to 50% in 7 min); Detector, UV 254/220 nm. The collected solution was dried by lyophilization. This resulted in 34 mg (54.6%) of 10-[3-(6-amino-5-2-[(2S)-1-propanoylpyrrolidin-2-yl]ethenyl]pyrimidin-4-yl)-2-methylphenyl]-4,4-dimethyl-1,10-diazatricyclo[6.4.0.0\hat{ }[2,6]]dodeca-2(6),7-dien-9-one as white solid. LC-MS (ESI, m/z) M+1: 539. 1 HNMR (300 MHz, DMSO-d₆) δ 8.30 (d, J=4.2 Hz, 1H), 7.32-7.20 (m, 2H), 7.02 (q, J=9.4, 8.5 Hz, 1H), 6.80 (s, 1H), 6.66 (s, 1H), 6.47 (d, J=3.5 Hz, 1H), 6.04-5.75 (m, 1H), (m, 1H), 4.43-4.25 (m, 1H), 4.20-4.14 (m, 1H), 4.21-4.03 (m, 3H), 3.72-3.68 (m, 1H), 3.21-2.95 (m, 2H), 2.55 (s, 2H), 2.41 (s, 2H), 2.24-2.04 (m, 1H), 1.88 (d, J=4.0 Hz, 3H), 1.79-1.34 (m, 2H), 1.21 (s, 6H), 0.97-0.81 (m, 3H).

### EXAMPLE 5

### Preparation of 10-[4-(6-amino-5-[[(2S)-1-(prop-2-enoyl)pyrrolidin-2-yl]methoxy]pyrimidin-4-yl)-3-(hydroxymethyppyridin-2-yl]-4,4-dimethyl-1,10-diazatricyclo [6.4.0.0\hat{ }[2,6] ]dodeca-2(6),7-dien-9-one

Synthesis of [(3,3-dimethylcyclopent-1-en-1-yl)oxy]trimethylsilane: Into a 10-L 4-necked round-bottom flask purged and maintained with an inert atmosphere of nitrogen, was placed CuCl (20.6 g, 208 mmol, 0.05 eq.), LiCl (17.6 g, 416.1 mmol, 0.1 eq.), tetrahydrofuran (2.5 L). This was followed by the addition of 2-cyclopenten-1-one, 3-methyl-(400.0 g, 4161.0 mmol, 1.0 eq.) at −5 to 5° C. To this was added TMSCl (474.7 g, 4369.1 mmol, 1.1 eq.) dropwise with stirring at −5 to 5° C. To the mixture was added MeMgCl (1670.0 mL, 14495.1 mmol, 3.5 eq.) dropwise with stirring at −5 to 10° C. The resulting solution was stirred for 2 hours at −5 to 10° C. in an ice/salt bath. The reaction was then quenched by the addition of MeOH (34 mL). The resulting solution was diluted with NH₄Cl (5 L). The solids were filtered out. The resulting solution was extracted with petroleum ether (3×5 L) and combined organic was dried over anhydrous sodium sulfate. After filtration, the filtrate was concentrated under reduced pressure. This resulted (780.2 g, crude) of [(3,3-dimethylcyclopent-1-en-1-yl)oxy]trimethylsilane as yellow oil. GC-MS (ES, m/z) M: 184.

Synthesis of 3,3-dimethylcyclopentanone: Into a 20-L 4-necked round-bottom flask, was placed [(3,3-dimethylcyclopent-1-en-1-yl)oxy]trimethylsilane (780.0 g, 4231.0 mmol, 1.0 eq.), CH₂Cl₂ (7.8 L), H₂O (30.5 g, 1692.4 mmol, 0.4 eq.). This was followed by the addition of POCl₃ (214.1 g, 1396.3 mmol, 0.3 eq.) dropwise with stirring at 25 to 30° C. The resulting solution was stirred for 0.5 hour at 25° C. This crude in solvent straight used for next step. GC-MS (ES, m/z) M: 112

Synthesis of 3,3-dimethylcyclopentanone: Into a 20-L 4-necked round-bottom flask, was placed previous step solution 3,3-dimethylcyclopentan-1-one in CH₂Cl₂ (7.80 L). This was followed by the addition of N,N-dimethylformamide (619.0 g, 8.5 mol, 2.0 eq.) dropwise with stirring at 25° C. To this was added POCl₃ (1362 g, 17.8 mol, 2.1 eq.) dropwise with stirring at 40° C. The resulting solution was stirred for overnight at 40° C. in an oil bath. The reaction was then quenched by the addition of K₃PO₄ (2000 g) in H₂O (5 L). The resulting solution was extracted with dichloromethane (3×10 L) and combined organic was dried over anhydrous sodium sulfate. After filtration, the filtrate was concentrated under reduced pressure. This resulted in This resulted (530 g, Crude) of 2-chloro-4,4-dimethylcyclopent-1-ene-1-carbaldehyde as a brown solid. GC-MS (ES, m/z): 158.

Synthesis of 4,4-dimethyl-1,10-diazatricyclo[6.4.0.0\hat{ }[2,6]]dodeca-2(6),7-dien-9-one: Into a 5-L 4-necked round-bottom flask, was placed 2-chloro-4,4-dimethylcyclopent-1-ene-1-carbaldehyde (474.0 g, 2988.1 mmol, 1.0 eq.), N,N-dimethylformamide (3 L), piperazin-2-one (299.2 g, 2988.1 mmol, 1.0 eq.), DIEA (463.4 g, 3585.7 mmol, 1.2 eq.). The resulting solution was stirred for overnight at 115° C. in an oil bath. The reaction mixture was cooled to 25° C. with a water/ice bath. The solids were collected by filtration. The resulting mixture was washed with H₂O (3×2 L) and petroleum ether (3×2L). The combined organic was dried over anhydrous sodium sulfate. After filtration, the filtrate was concentrated under reduced pressure. This resulted (30.0 g, 37.7%) of 4,4-dimethyl-1,10-diazatricyclo[6.4.0.0\hat{ }[2,6]]dodeca-2(6),7-dien-9-one as a grey solid.LC-MS (ES, m/z) M+1: 205.

Synthesis of 2,4-dibromopyridine-3-carbaldehyde: Into a 1000-mL 3-necked round-bottom flask, was placed 2,4-dibromopyridine (40.0 g, 168.9 mmol, 1.0eq.), THF (400 mL). This was followed by the addition of LDA (2M in hexane, 126 mL, 1.5 eq.) dropwise with stirring at −78° C. The resulting solution was stirred for 2 hours at −78° C. Then N,N-dimethylformamide (16.0 g, 219.5 mmol, 1.3eq.) was added dropwise with stirring at −78° C. The resulting solution was stirred for 1 hour at −78° C. The reaction mixture was quenched by NH₄lC/HOAc(1:1, 500 mL). The resulting solution was extracted with ethyl acetate (3×350 mL) and combined organic was dried over anhydrous sodium sulfate. After filtration, the filtrate was concentrated under reduced pressure. The crude residue was purified by a flash column (silica gel, ethyl acetate/petroleum ether=1:1). This resulted (24.4 g, 54.5%) of 2,4-dibromopyridine-3-carbaldehyde as white solid. LC-MS (ES, m/z) M+1: 264

Synthesis of (2,4-dibromopyridin-3-yl)methanol : Into a 100-mL round-bottom flask, was placed 2,4-dibromopyridine-3-carbaldehyde (2.0 g, 7.6 mmol, 1.0 eq.), EtOH (30 mL). This was followed by the addition of NaBH4 (286 mg, 7.6 mmol, 1.0 eq.), in portions at 0° C. The resulting solution was stirred for 3 hours at 0° C. The reaction mixture was quenched by the addition of H₂(30 mL). The resulting solution was extracted with ethyl acetate (3×30 mL) and combined organic was dried over anhydrous sodium sulfate. After filtration, the filtrate was concentrated under reduced pressure. The crude residue was purified by a flash column (silica gel, ethyl acetate/petroleum ether=1:1). This resulted (1.4 g, 69.5%) of (2,4-dibromopyridin-3-yl)methanol as light yellow solid. LC-MS (ES, m/z) M+1: 266.

Synthesis of 2,4-dibromo-3-[(oxan-2-yloxy)methyl]pyridine: Into a 100-mL round-bottom flask, was placed (2,4-dibromopyridin-3-yl)methanol (1.4 g, 5.2 mmol, 1.0 eq.), CH₂Cl₂ (30 mL), PPTS (132 mg, 0.5 mmol, 0.1eq.), DHP (662 mg, 7.9 mmol, 1.5 eq.). The resulting solution was stirred for overnight at 45° C. in an oil bath. The reaction mixture was cooled to 25° C. The reaction was then quenched by H₂O (30 mL). The resulting solution was extracted with dichloromethane (3×30 mL) and combined organic was dried over anhydrous sodium sulfate. After filtration, the filtrate was concentrated under reduced pressure. The crude residue was purified by a flash column (silica gel, ethyl acetate/petroleum ether=1:1). This resulted (1.5 g, 80.0%) of 2,4-dibromo-3-[(oxan-2-yloxy)methyl]pyridine as colorless oil. LC-MS (ES, m/z) M+1: 350.

Synthesis of 10-[4-bromo-3-Roxan-2-yloxy)methyllpyridin-2-yl]-4,4-dimethyl-1,10-diazatricyclo[6.4.0.0\hat{ }[2,6]]dodeca-2(6),7-dien-9-one: Into a 100-mL round-bottom flask purged and maintained with an inert atmosphere of nitrogen, was placed 4,4-dimethyl-1,10-diazatricyclo[6.4.0.0\hat{ }[2,6]]dodeca-2(6),7-dien-9-one (1.0 g, 4.9 mmol, 1.0 eq.), dioxane (40 mL), Cs₂CO₃ (3.2 g, 9.8 mmol, 2 eq.), 2,4-dibromo-3-[(oxan-2-yloxy)methyl]pyridine (1.7 g, 4.9 mmol, 1.0 eq.), Pd₂(dba)₃(448 mg, 0.5 mmol, 0.1 eq.), XantPhos (283 mg, 0.5 mmol, 0.1eq.). The resulting solution was stirred for 3 hours at 100° C. in an oil bath. The reaction mixture was cooled to 25° C. and quenched by the addition of water (40 mL). The resulting solution was extracted with ethyl acetate (3×40 mL) and combined organic was dried over anhydrous sodium sulfate. After filtration, the filtrate was concentrated under reduced pressure. The crude residue was purified by a flash column (silica gel, ethyl acetate/petroleum ether=1:1). This resulted (900 mg, 38.7%) of 10-[4-bromo-3-[(oxan-2-yloxy)methyl]pyridin-2-yl]-4,4-dimethyl-1,10-diazatricyclo[6.4.0.0\hat{ }[2,6]]dodeca-2(6),7-dien-9-one as brown solid. LC-MS (ES, m/z) M+1: 474

Synthesis of 2-[4,4-dimethyl-9-oxo-1,10-diazatricyclo[6.4.0.0\hat{ }[2,6]]dodeca-2(6),7-dien-10-yl]-3-Roxan-2-yloxy)methyllpyridin-4-ylboronic acid: Into a 100-mL round-bottom flask purged and maintained with an inert atmosphere of nitrogen, was placed 104-[4-bromo-3-[(oxan-2-yloxy)methyl]pyridin-2-yl]-4,4-dimethyl-1,10-diazatricyclo[6.4.0.0\hat{ }[2,6]]dodeca-2(6),7-dien-9-one (1.0 g, 2.1 mmol, 1.0 eq.), dioxane (10 mL), bis(pinacolato)diboron (1.3 g, 5.3 mmol, 2.5 eq.), KOAc (620 mg, 6.3 mmol, 3.0 eq.), Pd(dppf)Cl₂ (172 mg, 0.2 mmol, 0.1 eq.). The resulting solution was stirred for 2 hours at 100° C. in an oil bath. The reaction mixture was cooled to 25° C. The solids were filtered out. The resulting mixture was concentrated under vacuum. This resulted (920 mg, crude) of 2-[4,4-dimethyl-9-oxo-1,10-diazatricyclo [6.4.0.0\hat{ }[2,6]]dodeca-2(6),7-dien-10-yl]-3-[(oxan-2-yloxy)methyl]pyridin-4-ylboronic acid as brown oil. LC-MS (ES, m/z) M+1: 440.

Synthesis of 10-[1-hydroxy-3H-[1,2]oxaborolo[4,3-c]pyridin-4-yl]-4,4-dimethyl-1,10-diazatricyclo[6.4.0.0\hat{ }[2,6]]dodeca-2(6),7-dien-9-one: Into a 100-mL round-bottom flask, was placed 2-[4,4-dimethyl-9-oxo-1,10-diazatricyclo[6.4.0.0\hat{ }[2,6]]dodeca-2(6),7-dien-acid (920 mg, 1.0 eq., crude), dioxane (10 mL), HCl (6N, 10 mL). The resulting solution was stirred for 1 hour at 25° C. The resulting mixture was concentrated under vacuum. The crude product was purified by Flash-Prep-HPLC with the following conditions: Column, C18 reversed phase column; mobile phase, H₂O(0.05% NH_{3.}H₂O) and CH3CN (5% CH₃CN up to 30% in 15 minutes); Flow rate: mL/minutes; Detector, 254/220 nm. This resulted (350 mg) of 10-1-hydroxy-3H-[1,2]oxaborolo[4,3-c]pyridin-4-yl]-4,4-dimethyl-1,10-diazatricyclo[6.4.0.0\hat{ }[2,6] ]dodeca-2(6),7-dien-9-one as light yellow solid.LC-MS (ES, m/z) M+1: 338.

Synthesis of 6-chloro-5-methoxypyrimidin-4-amine: Into a 500 mL pressure tank reactor, was placed 4,6-dichloro-5-methoxypyrimidine (50.0 g, 279.3 mmol, 1.0 eq.). This was followed by the addition of Ammonia 3M in MeOH (250 mL) at 25° C. The resulting solution was stirred for overnight at 100° C. The reaction mixture was cooled to 25° C. The resulting mixture was concentrated. The crude residue was purified by a flash column (silica gel, ethyl acetate/petroleum ether=1:10-2:1). This resulted in 30 g (67.3%) of 6-chloro-5-methoxypyrimidin-4-amine as white solid. 1 HNMR (300 MHz, DMSO-d₆) δ 7.97 (s, 1H), 7.29 (s, 2H), 3.71 (s, 3H).

Synthesis of 4-amino-6-chloropyrimidin-5-ol: Into a 1000 mL 3-necked round-bottom flask, was placed 6-chloro-5-methoxypyrimidin-4-amine (30.0 g, 188.0 mmol, 1.0 eq.), CH₂Cl₂ (60 mL). This was followed by the addition of boron tribromide 1M in CH₂Cl₂ (375 mL, 375.0 mmol, 2.0 eq.) at 25° C. The resulting solution was stirred for 4 hours at 40° C. The reaction mixture was cooled to 25° C. The reaction was then quenched by the addition of 100 mL of MeOH. The resulting solution was diluted with 300 mL of H₂O. The pH value of the solution was adjusted to 8 with NaOH (2 mol/L). The solids were collected by filtration. This resulted in 15 g (54.8%) of 4-amino-6-chloropyrimidin-5-ol as white solid. LC-MS (ESI, m/z) M+1:146. 1 HNMR (300 MHz, DMSO-d₆) 6 9.58 (s, 1H), 7.79 (s, 1H), 6.91 (s, 2H).

Synthesis of tert-butyl (2S)-2-[[4-amino-6-chloropyrimidin-5-yl)oxy]methyl]pyrrolidine-1-carboxylate: Into a 250 mL 3-necked round-bottom flask, was placed 4-amino-6-chloropyrimidin-5-ol (4.0 g, 27.5 mmol, 1.0 eq.), tert-butyl (2S)-2-(hydroxymethyl)pyrrolidine-1-carboxylate (6.6 g, 33.0 mmol, 1.2 eq.), PPh₃ (8.6 g, 33.0 mmol, 1.2 eq.), tetrahydrofuran (80 mL). This was followed by the addition of DIAD (6.6 g, 33.0 mmol, 1.2 eq.) dropwise with stirring at 0° C. The resulting solution was stirred for 2 hours at 25° C. The resulting solution was diluted with 100 mL of H₂O. The resulting solution was extracted with 2×100 mL of ethyl acetate and the organic layers combined. The resulting mixture was washed with 100 mL of brine. The mixture was dried over anhydrous sodium sulfate. After filtration, the filtrate was concentrated under reduced pressure. The crude residue was purified by a flash column (silica gel, ethyl acetate/petroleum ether=1:5-1:1). This resulted in 1.6 g (17.7%) of tert-butyl (2S)-2-[[(4-amino-6-chloropyrimidin-5-yl)oxy]methyl]pyrrolidine-1-carboxylate as colorless oil. LC-MS (ESI, m/z) M+1: 329.

Synthesis of tert-butyl (2S)-2-([[4-amino-6-(2-[4,4-dimethyl-9-oxo-1,10-diazatricyclo [6.4.0.0\hat{ }[2,6] ] dodeca-2(6),7-dien-10-yl]-3-(hydroxymethyl)pyridin-4-yl)pyrimidin-5-yl]oxy]methyppyrrolidine-1-carboxylate: Into a 100 mL round-bottom flask purged and maintained with an inert atmosphere of nitrogen, was placed tert-butyl (2S)-2-[[(4-amino-6-chloropyrimidin-5-yl)oxy]methyl]pyrrolidine-1-carboxylate (500.0 mg, 1.5 mmol, 1.0 eq.), 10-[1-hydroxy-3H-[1,2]oxaborolo[4,3-c]pyridin-4-yl]-4,4-dimethyl-1,10-diazatricyclo[6.4.0.0\hat{ }[2,6]]dodeca-2(6),7-dien-9-one (564.0 mg, 1.6 mmol, 1.1 eq.), DME (5 mL), H₂O (0.5 mL), K₂CO₃ (420.3 mg, 3.0 mmol, 2.0 eq.), Pd(PPh₃)₂Cl₂. CH₂Cl₂ (139.0 mg, mmol, 0.1 eq.). The resulting solution was stirred for 2 hours at 110° C. The reaction mixture was cooled to 25° C. The resulting solution was diluted with 100 mL of ethyl acetate and 50 mL of H₂O. The resulting solution was extracted with 2×50 mL of ethyl acetate and the organic layers combined. The resulting mixture was washed with 1×50 mL of brine. The mixture was dried over anhydrous sodium sulfate. After filtration, the filtrate was concentrated under reduced pressure. The crude residue was purified by a flash column (silica gel, ethyl acetate/petroleum ether=1:7-2:1). This resulted in 270 mg (29.4%) of tert-butyl (2S)-2-([[4-amino-6-(2-[4,4-dimethyl-9-oxo-1,10-diazatricyclo[6.4.0.0\hat{ }[2,6]]dodeca-2(6),7-dien-10-yl]-3-(hydroxymethyl)pyridin-4-yl)pyrimidin-5-yl]oxy]methyl)pyrrolidine-1-carboxylate as light yellow oil. LC-MS (ESI, m/z) M+1:604.

Synthesis of 10-(4-[6-amino-5-[(2S)-pyrrolidin-2-ylmethoxy]pyrimidin-4-yl]-3-(hydroxymethyppyridin-2-yl)-4,4-dimethyl-1,10-diazatricyclo[6.4.0.0\hat{ }[2,6] ]dodeca-2(6),7-dien-9-one: Into a 100 mL round-bottom flask, was placed tert-butyl (2S)-2-([[4-amino-6-(2-[4,4-dimethyl-9-oxo-1,10-diazatricyclo [6.4.0.0\hat{ }[2,6] ] dodeca-2 (6),7-dien-10-yl] -3-(hydroxymethyl)pyridin-4-yl)pyrimidin-5-yl]oxy]methyl)pyrrolidine-1-carboxylate (260.0 mg, 0.4 mmol, 1.0 eq.), CH₂Cl₂ (3 mL), TFA (1 mL). The resulting solution was stirred for 2 hours at 25° C. The resulting mixture was concentrated. This resulted in 160 mg (73.7%) of 2-yl)-4,4-dimethyl-1,10-diazatricyclo[6.4.0.0\hat{ }[2,6]]dodeca-2(6),7-dien-9-one as yellow solid. LC-MS (ESI, m/z) M+1: 504.

Synthesis of 10-[4-(6-amino-5-[[(2S)-1-(prop-2-enoyl)pyrrolidin-2-yl]methoxy]pyrimidin-4-yl)-3-(hydroxymethyppyridin-2-yl]-4,4-dimethyl-1,10-diazatricyclo[6.4.0.0\hat{ }[2,6]]dodeca-2(6),7-dien-9-one: Into a 8-mL vial, was placed 10-(4-[6-amino-S-[(2S)-pyrrolidin-2-ylmethoxy]pyrimidin-4-yl]-3-(hydroxymethyl)pyridin-2-yl)-4,4-dimethyl-1,10-diazatricyclo[6.4.0.0\hat{ }[2,6]]dodeca-2(6),7-dien-9-one (160.0 mg, 0.3 mmol, 1.0 eq.), CH₂Cl₂ (2 mL), trimethylamine (64.3 mg, 0.6 mmol, 2 eq.). This was followed by the addition of a solution of acryloyl chloride (28.7 mg, 0.3 mmol, 1.0 eq.) in CH₂Cl₂ (0.2 mL) dropwise with stirring at 0° C. The resulting solution was stirred for 2 hours at 25° C. The resulting mixture was concentrated. The crude product was purified by Prep-HPLC with the following conditions: Column, X Bridge Shield RP18 OBD Column, Sum, 19*150 mm; mobile phase, H₂O (0.05% TFA) and CH₃CN (26% Phase B up to 50% in 7 min); Detector, UV 254/220 nm. The collected solution was concentrated under vacumn to remove CH₃CN and the resulting solution was dried by lyophilization). This resulted in 64 mg (36.1%) of 10-[4-(6-amino-5-[[(2S)-1-(prop-2-enoyl)pyrrolidin-2-yl]methoxy]pyrimidin-4-yl)-3-(hydroxymethyl)pyridin-2-yl]-4,4-dimethyl-1,10-diazatricyclo[6.4.0.0\hat{ }[2,6]]dodeca-2(6),7-dien-9-one as white solid. LC-MS (ESI, m/z) M+1:558. 1 HNMR (300 MHz, DMSO-d6) 6 8.48 (d, J=4.5 Hz, 1H), 8.20 (d, J=5.8 Hz, 1H), 7.30 (dd, J=17.8, 5.0 Hz, 1H), 7.14 (d, J=25.7 Hz, 1H), 6.55 (d, J=2.1 Hz, 1H), 6.10 (d, J=16.3 Hz, 1H), 5.65 (d, J=13.7 Hz, 1H), 4.68 (s, 1H), 4.38 (d, J=5.7 Hz, 1H), 4.20 (s, 3H), 3.89 (s, 1H), 3.74-3.58 (m, 1H), 3.54-3.12 (m, 7H), 2.58 (d, J=4.4 Hz, 2H), 2.43 (s, 2H), 1.66 (d, J=20.2 Hz, 4H), 1.22 (s, 6H).

### EXAMPLE 6

### Preparation of (S)-2-(3-(5-(2-(1-acryloylpyrrolidin-2-yl)vinyl)-6-aminopyrimidin-4-yl)-5-fluoro-2-methylphenyl)-7,7-dimethyl-3,4,7,8-tetrahydro-2H-cyclopenta [4,5] pyrrolo [1,2-a]pyrazin-1 (6H)-one

Synthesis of [(3,3-dimethylcyclopent-1-en-1-yl)oxy]trimethylsilane: Into a 10 L 4-necked round-bottom flask purged and maintained with an inert atmosphere of nitrogen, was placed CuCl (20.6 g, 208.0 mmol, 0.05 eq.), LiCl (17.6 g, 416.1 mmol, 0.1 eq.), tetrahydrofuran (2.5 L). This was followed by the addition of 2-cyclopenten-1-one, 3-methyl-(400.0 g, 4161.0 mmol, 1.0 eq.) at −5 to 5° C. To this was added TMSCl (474.7 g, 4369.1 mmol, 1.1 eq.) dropwise with stirring at −5 to 5° C. To the mixture was added MeMgCl (1670.0 mL, 14495.1 mmol, 3.5 eq.) dropwise with stirring at −5 to 10° C. The resulting solution was stirred for 2 hours at −5 to 10° C. in an ice/salt bath. The reaction was then quenched by the addition of MeOH (34 mL). The resulting solution was diluted with NH₄Cl (5 L). The solids were filtered out. The resulting solution was extracted with petroleum ether (3×5 L) and combined organic was dried over anhydrous sodium sulfate. After filtration, the filtrate was concentrated under reduced pressure. This resulted (780.2 g, crude) of [(3,3-dimethylcyclopent-1-en-1-yl)oxy]trimethylsilane as yellow oil. GC-MS (ES, m/z) M: 184.

Synthesis of 3,3-dimethylcyclopentanone: Into a 20 L 4-necked round-bottom flask, was placed [(3,3-dimethylcyclopent-1-en-1-yl)oxy]trimethylsilane (780.0 g, 4231.0 mmol, 1.0 eq.), CH₂Cl₂ (7.8 L), H₂O (30.5 g, 1692.4 mmol, 0.4 eq.). This was followed by the addition of POCl₃ (214.1 g, 1396.3 mmol, 0.3 eq.) dropwise with stirring at 25 to 30° C. The resulting solution was stirred for 0.5 hour at 25° C. This crude in solvent straight used for next step. GC-MS (ES, m/z) M: 112.

Synthesis of 3,3-dimethylcyclopentanone: Into a 20 L 4-necked round-bottom flask, was placed previous step solution 3,3-dimethylcyclopentan-1-one in CH₂Cl₂ (7.8 L). This was followed by the addition of N,N-dimethylformamide (619.0 g, 8.5 mol, 2.0 eq.) dropwise with stirring at 25° C. To this was added POCl₃ (1362.0 g, 17.8 mol, 2.1 eq.) dropwise with stirring at 40° C. The resulting solution was stirred for overnight at 40° C. in an oil bath. The reaction was then quenched by the addition of K3PO4 (2000 g) in H₂O (5 L). The resulting solution was extracted with dichloromethane (3×10 L) and combined organic was dried over anhydrous sodium sulfate. After filtration, the filtrate was concentrated under reduced pressure. This resulted in This resulted (530 g, Crude) of 2-chloro-4,4-dimethylcyclopent-1-ene-1-carbaldehyde as a brown solid. GC-MS (ES, m/z): 158.

Synthesis of 4,4-dimethyl-1,10-diazatricyclo[6.4.0.0\hat{ }[2,6]]dodeca-2(6),7-dien-9-one: Into a 5 L 4-necked round-bottom flask, was placed 2-chloro-4,4-dimethylcyclopent-1-ene-1-carbaldehyde (474.0 g, 2988.1 mmol, 1.0 eq.), N,N-dimethylformamide (3 L), piperazin-2-one (299.2 g, 2988.1 mmol, 1.0 eq.), DIEA (463.4 g, 3585.7 mmol, 1.2 eq.). The resulting solution was stirred for overnight at 115° C. in an oil bath. The reaction mixture was cooled to 25° C. with a water/ice bath. The solids were collected by filtration. The resulting mixture was washed with H₂O (3×2 L) and petroleum ether (3×2 L). The combined organic was dried over anhydrous sodium sulfate. After filtration, the filtrate was concentrated under reduced pressure. This resulted (30.0 g, 37.7%) of 4,4-dimethyl-1,10-diazatricyclo[6.4.0.0\hat{ }[2,6]]dodeca-2(6),7-dien-9-one as grey solid. LC-MS (ES, m/z) M+1: 205.

Synthesis of 2-(3-bromo-5-fluoro-2-methylphenyl)-7,7-dimethyl-3,4,7,8-tetrahydro-2H-cyclopenta[4,5]pyrrolo[1,2-a]pyrazin-1(6H)-one: Into a 250 mL 3-necked round-bottom flask with DMA (100.00mL) were added 1,3-dibromo-5-fluoro-2-methylbenzene (5.0 g, 18.7 mmol, 1.0 eq.) and 7,7-dimethyl-3,4,7,8-tetrahydro-2H-cyclopenta[4,5]pyrrolo[1,2-a]pyrazin-1(6H)-one (4.6 g, 22.4 mmol, 1.2 eq.) at 25° C. To the above mixture was added Cul (2.8 g, 14.9 mmol, 0.8 eq.), K2CO3(7.7g, 56.0mmol, 3.0 eq.) and 1,10-phenanthroline (2.0 g, 11.2 mmol, 0.6 eq.) at 25° C. The resulting mixture was stirred for 16 hours at 100° C. The resulting mixture was quenched with water (200 mL). The resulting mixture was extracted with EtOAc (1×200 mL). The combined organic layers were washed with brine (1×200 mL), dried over anhydrous Na2SO4. After filtration, the filtrate was concentrated under reduced pressure. The residue was purified by silica gel column chromatography, eluted with CH₂Cl₂/EtOAc (1:1) to afford (1.2 g, 16.4%) 2-(3-bromo-5-fluoro-2-methylphenyl)-7,7-dimethyl-3,4,7,8-tetrahydro-2H-cyclopenta[4,5]pyrrolo[1,2-a]pyrazin-1(6H)-one as a white solid. LC-MS: (ESI, m/z): M+1: 391/393.

Synthesis of 2-(5-fluoro-2-methyl-3-(4,4,5,5-tetramethyl-1,3,2-dioxaborolan-2-yl)phenyl)-7,7-dimethyl-3,4,7,8-tetrahydro-2H-cyclopenta[4,5]pyrrolo[1,2-a]pyrazin-1(6H)-one: Into a 100 mL 3-necked round-bottom flask purged and maintained with an inert atmosphere of nitrogen, was placed 2-(3-bromo-5-fluoro-2-methylphenyl)-7,7-dimethyl- 3,4,7,8-tetrahydro-2H-cyclopenta[4,5]pyrrolo[1,2-a]pyrazin-1(6H)-one (1.2 g, 3.1 mmol, 1.0 eq.), bis(pinacolato)diboron (1.6 g, 6.1 mmol, 2.0 eq.), KOAc (903 mg, 9.2 mmol, 3.0 eq.), Pd(dppf)Cl₂ (250 mg, 0.3 mmol, 0.1 eq.), dioxane (20 mL). The resulting solution was stirred for 16 hours at 100° C. in an oil bath. The reaction was then quenched by the addition of 30 mL of water. The resulting solution was extracted with ethyl acetate (3×50 mL). The combined organic layers were washed with brine (1×200 mL), dried over anhydrous Na₂SO₄. After filtration, the filtrate was concentrated under reduced pressure. The residue was applied onto a silica gel column and eluted with ethyl acetate/petroleum ether (1:3). The collected fractions were combined and concentrated. This resulted (210 mg, 15.6%) of 2-(5-fluoro-2-methyl-3-(4,4,5,5-tetramethyl-1,3,2-dioxaborolan-2-yl)phenyl)-7,7-dimethyl-3,4,7,8-tetrahydro-2H-cyclopenta[4,5]pyrrolo[1,2-a]pyrazin-1(6H)-one as a white solid. LC-MS: (ESI, m/z): M+1: 439

Synthesis of 2-(dichloromethyl)-4,4,5,5-tetramethyl-1,3,2-dioxaborolane: Into a 1 L round-bottom flask, was placed methylene chloride (46.2 g, 543.9 mmol, 1.0 eq.), THF (500 mL). This was followed by the addition of 2.5 M n-BuLi (217 mL, 543.9 mmol, 1.0 eq.) dropwise with stirring at −100° C. in 30 min. To this was added trimethyl borate (56.5 g, 543.9 mmol, 1.0 eq.) at −100° C., the resulting solution was stirred for 1 hour at −100° C. The reaction was then quenched by the addition of 100 mL of HCl (5 M). The resulting solution was extracted with of Et2O (3×200 mL) and the combined organic phase was dried over anhydrous magnesium sulfate. After filtration, the filtrate was concentrated under reduced pressure. This was followed by addition of benzene (500 mL), pinacol (64.3 g, 544.0 mmol, 1.0 eq.). The resulting solution was stirred while the temperature was maintained at 90° C. in an oil bath. The crude product was purified by distillation under reduced pressure (0.1MPa) and the fraction was collected at 130° C. This resulted (48.0 g, 41.8%) of 2-(dichloromethyl)-4,4,5,5-tetramethyl-1,3,2-dioxaborolane as white solid. ¹HNMR (300 MHz, DMSO-d₆) δ 5.34 (s, 1H), 1.12 (s, 12H).

Synthesis of tert-butyl (2S)-2-formylpyrrolidine-1-carboxylate: Into a 250 mL round-bottom flask, was placed tert-butyl (2S)-2-(hydroxymethyl)pyrrolidine-1-carboxylate (8.0 g, 39.7mmol, 1.0 eq.), CH₂Cl₂(100 mL), DMP (30.3 g, 71.5 mmol, 1.8 eq.). The resulting solution was stirred for overnight at 25° C. The solids were filtered out. The resulting solution was extracted with dichloromethane (3×100 mL) and the combined organic phase was dried over anhydrous magnesium sulfate. After filtration, the filtrate was concentrated under reduced pressure. The crude residue was purified by a flash column (silica gel, ethyl acetate/petroleum ether=1:3). The collected fractions were combined and concentrated. This resulted (4.5 g, 56.8%) of tert-butyl (2S)-2-formylpyrrolidine-1-carboxylate as light yellow oil. LC-MS (ESI, m/z) M+1: 200.

Synthesis of tert-butyl (2S)-2-[2-(4,4,5,5-tetramethyl-1,3,2-dioxaborolan-2-yl)ethenyl]pyrrolidine-1-carboxylate: Into a 250 mL 3-necked round-bottom flask purged and maintained with an inert atmosphere of nitrogen, was placed CrCl₃ (15.9 g, 129.7 mmol, 8.0 eq.), tetrahydrofuran (130 mL), tert-butyl (2S)-2-formylpyrrolidine-1-carboxylate (3.2 g, 16.2mmol, 1.0 eq.), 2-(dichloromethyl)-4,4,5,5-tetramethyl-1,3,2-dioxaborolane (6.8 g, 32.4 mmol, 2.0 eq.). This was followed by the addition of LiI (8.7 g, 64.8 mmol, 4.0 eq.) in tetrahydrofuran (10 mL) dropwise with stirring at 25° C. The resulting solution was stirred for overnight at 25° C. The reaction was then quenched by the addition of 100 mL of water/ice. The resulting solution was extracted with dichloromethane (3×100 mL) and the combined organic phase was dried over anhydrous magnesium sulfate. After filtration, the filtrate was concentrated under reduced pressure. The crude residue was purified by a flash column (silica gel, ethyl acetate/petroleum ether=1:10). The collected fractions were combined and concentrated. This resulted (1.9 g, 36.2%) of tert-butyl (2S)-2-[2-(4,4,5,5-tetramethyl-1,3,2-dioxaborolan-2-yl)ethenyl]pyrrolidine-1-carboxylate as yellow oil. LC-MS (ESI, m/z) M+1: 324.

Synthesis of tert-butyl (2S)-2-[2-(4-amino-6-chloropyrimidin-5-ypethenylipyrrolidine-1-carboxylate: Into a 100 mL round-bottom flask purged and maintained with an inert atmosphere of nitrogen, was placed tert-butyl (2S)-2-[2-(4,4,5,5-tetramethyl-1,3,2-dioxaborolan-2-yl)ethenyl]pyrrolidine-1-carboxylate (2.4 g, 7.5 mmol, 1.0 eq.), DME (50 mL), H₂O (5 mL), 6-chloro-5-iodopyrimidin-4-amine (1.9 g, 7.5 mmol, 1.0 eq.), Na₂CO₃ (2.4 g, 22.5 mmol, 3.0 eq.), Pd(PPh)₃Cl₂ (527.6 mg, 0.7 mmol, 0.1 eq.). The resulting solution was stirred for overnight at 80° C. in an oil bath. The reaction was cooled to 25° C. and then quenched by the addition of 50 mL of H₂O. The resulting solution was extracted with ethyl acetate (3×70 mL), and the combined organic phase was washed by brine (1×70 mL), the mixture was dried by Na₂SO₄ and filtrated, the filtrate was concentrated. The crude residue was purified by a flash column (silica gel, ethyl acetate/petroleum ether=1:1). The collected fractions were combined and concentrated. This resulted (1.2 g, 49.1%) of tert-butyl (2S)-2-2-(4-amino-6-chloropyrimidin-5-yl)ethenyl]pyrrolidine-1-carboxylate as white solid. LC-MS (ESI, m/z) M+1: 325.

Synthesis of tert-butyl (S)-2-(2-(4-amino-6-(3-(7,7-dimethyl-1-oxo-1,3,4,6,7,8-hexahydro-2H-cyclopenta[4,5]pyrrolo[1,2-a]pyrazin-2-yl)-5-fluoro-2-methylphenyl)pyrimidin-5-yl)vinyl)pyrrolidine-1-carboxylate: Into a 50-mL round-bottom flask purged and maintained with an inert atmosphere of nitrogen, was placed tert-butyl (S)-2-(2-(4-amino-6-chloropyrimidin-5-yl)vinyl)pyrrolidine-1-carboxylate (150 mg, 0.4 mmol, 1.0 eq.), 2-(5-fluoro-2-methyl-3-(4,4,5,5-tetramethyl-1,3,2-dioxaborolan-2-yl)phenyl)-7,7-dimethyl-3,4,7,8-tetrahydro-2H-cyclopenta[4,5]pyrrolo[1,2-a]pyrazin-1(6H)-one(200 mg, 0.4 mmol, 1.0 eq.), K₂CO₃ (189 mg, 1.2 mmol, 3.0 eq.), Pd(PPh₃)₂Cl₂(32 mg, 0.04 mmol, 0.1 eq.), DME (5 mL), H₂O (0.5 mL). The resulting solution was stirred for 2 hours at 110° C. in an oil bath. The resulting solution was cooled to 25° C. and diluted with H₂O (15 mL). The resulting solution was extracted with ethyl acetate (3×20 mL). The combined organic layers were washed with brine (1×300 mL), dried over anhydrous Na₂SO₄. After filtration, the filtrate was concentrated under reduced pressure. The residue was applied onto a silica gel column and eluted with ethyl acetate/petroleum ether (1:1). The collected fractions were combined and concentrated. This resulted in tert-butyl (S)-2-(2-(4-amino-6-(3-(7,7-dimethyl-1-oxo-1,3,4,6,7,8-hexahydro-2H-cyclopenta[4,5]pyrrolo[1,2-a]pyrazin-2-yl)-5-fluoro-2-methylphenyl)pyrimidin-5-yl)vinyl)pyrrolidine-1-carboxylate (100 mg, 36.3%) as a white solid. LC-MS: (ESI, m/z): M+1: 605.

Synthesis of (S)-2-(3-(6-amino-5-(2-(pyrrolidin-2-yl)vinyl)pyrimidin-4-yl)-5-fluoro-2-methylphenyl)-7,7-dimethyl-3,4,7,8-tetrahydro-2H-cyclopenta[4,5]pyrrolo[1,2-a]pyrazin-1(6H)-one hydrochloride: Into a 50-mL round-bottom flask, was placed tert-butyl (S)-2-(((4-(3-(4-cyclopropyl-2-fluorobenzamido)-5-fluoro-2-methylphenyl)-6-(methylamino)pyrimidin-5-yl)oxy)methyl)pyrro lidine-1-carboxylate (100 mg, 0.2 mmol, 1.0 eq.), HCl (gas) in 1,4-dioxane (5 mL). The resulting solution was stirred for 2 hours at 25° C. The resulting mixture was concentrated. This resulted (80 mg, crude) of (S)-2-(3-(6-amino-5-(2-(pyrrolidin-2-yl)vinyl)pyrimidin-4-yl)-5-fluoro-2-methylphenyl)-7,7-dimethyl-3,4,7,8-tetrahydro-2H-cyclopenta[4,5]pyrrolo[1,2-a]pyrazin-1(6H)-one hydrochloride e as a white solid.LC-MS (ESI, m/z) M+1: 505

Synthesis of (S)-2-(3-(5-(2-(1-acryloylpyrrolidin-2-yl)vinyl)-6-aminopyrimidin-4-yl)-5-fluoro-2-methylphenyl)-7,7-dimethyl-3,4,7,8-tetrahydro-2H-cyclopental4,5pyrrolo[,1,2-a]pyrazin-1(6H)-one: Into a 8 mL vial, was placed (S)-2-(3-(6-amino-5-(2-(pyrrolidin-2-yl)vinyl)pyrimidin-4-yl)-5-fluoro-2-methylphenyl)-7,7-dimethyl-3,4,7,8-tetrahydro-2H-cyclopenta[4,5]pyrrolo[1,2-a]pyrazin-1(6H)-one hydrochloride e (60 mg, 0.1 mmol, 1.0 eq.), CH₂Cl₂ (1 mL), trimethylamine (23 mg, 0.2 mmol, 2.0 eq.). This was followed by the addition of a solution of acryloyl chloride (10 mg, 0.1 mmol, 1.0 eq.) in CH₂Cl₂ (0.2 mL) dropwise with stirring at 25° C. The resulting solution was stirred for 2 hours at 25° C. The resulting mixture was concentrated. The crude product was purified by Prep-HPLC with the following conditions: Column, X Bridge Shield RP18 OBD Column, 19*150 mm; mobile phase, H₂O (0.05% TFA) and CH3CN (26% Phase B up to 50% in 7 min); Detector, UV 254/220 nm. The collected solution was dried by lyophilization. This resulted (31 mg,49.9%) of (S)-2-(3-(5-(2-(1-acryloylpyrrolidin-2-yl)vinyl)-6-aminopyrimidin-4-yl)-5-fluoro-2-methylphenyl)-7,7-dimethyl-3,4,7,8-tetrahydro-2H-cyclopenta[4,5]pyrrolo[1,2-a]pyrazin-1(6H)-one as white solid. LC-MS (ESI, m/z) M+1: 555. ¹HNMR (300 MHz, DMSO-d₆) δ 8.30 (s, 1H), 7.28-7.25 (m, 1H), 6.95-6.89 (m, 3H), 6.58-6.55 (m, 1H), 6.50 (s, 1H), 6.02-5.58 (m, 2H),5.50-5.46 (m, 2H), 4.43 (d, J=27.8 Hz, 2H), 4.23-3.98 (m, 3H), 3.70 (s, 1H), 3.46-3.14 (m, 2H), 2.55 (s, 2H), 2.41 (s, 2H), 1.86 (d, J=16.2 Hz, 3H), 1.64 (s, 1H), 1.55 (s, 1H), 1.44 (s, 1H), 1.21 (s, 6H).

### EXAMPLE 7

### Preparation of 10-[5-fluoro-2-methyl-3-(5-{[(2S)-1-(prop-2-enoyl)pyrrolidin-2-yl]methoxy}pyrimidin-4-yl)phenyl]-4,4-dimethyl-1,10-diazatricyclo[6.4.0.0\hat{ }{2,6}]dodeca-2(6),7-dien-9-one

Synthesis of [(3,3-dimethylcyclopent-1-en-1-yl)oxy]trimethylsilane: Into a 10 L 4-necked round-bottom flask purged and maintained with an inert atmosphere of nitrogen, was placed CuCl (20.6 g, 208 mmol, 0.05 eq.), LiCl (17.6 g, 416.1 mmol, 0.1 eq.), tetrahydrofuran (2.5 L). This was followed by the addition of 2-cyclopenten-1-one, 3-methyl-(400.0 g, 4161.0 mmol, 1.0 eq.) at −5 to 5° C. To this was added TMSCl (474.7 g, 4369.1 mmol, 1.1 eq.) dropwise with stirring at −5 to 5° C. To the mixture was added MeMgCl (1670.0 mL, 14495.1 mmol, 3.5 eq.) dropwise with stirring at −5 to 10° C. The resulting solution was stirred for 2 hours at −5 to 10° C. in an ice/salt bath. The reaction was then quenched by the addition of MeOH (34 mL). The resulting solution was diluted with NH₄Cl (5 L). The solids were filtered out. The resulting solution was extracted with petroleum ether (3×5 L) and combined organic was dried over anhydrous sodium sulfate. After filtration, the filtrate was concentrated under reduced pressure. This resulted (780.2 g, crude) of [(3,3-dimethylcyclopent-1-en-1-yl)oxy]trimethylsilane as yellow oil. GC-MS (ES, m/z) M: 184.

Synthesis of 3,3-dimethylcyclopentanone: Into a 20 L 4-necked round-bottom flask, was placed [(3,3-dimethylcyclopent-1-en-1-yl)oxy]trimethylsilane (780.0 g, 4231.0 mmol, 1.0 eq.), CH₂Cl₂ (7.8 L), H₂O (30.5 g, 1692.4 mmol, 0.4 eq.). This was followed by the addition of POCl₃ (214.1 g, 1396.3 mmol, 0.3 eq.) dropwise with stirring at 25 to 30° C. The resulting solution was stirred for 0.5 hour at 25° C. This crude in solvent straight used for next step.GC-MS (ES, m/z) M: 112.

Synthesis of 3,3-dimethylcyclopentanone: Into a 20-L 4-necked round-bottom flask, was placed previous step solution 3,3-dimethylcyclopentan-1-one in CH₂Cl₂ (7.80 L). This was followed by the addition of N,N-dimethylformamide (619.0 g, 8.5 mol, 2.0 eq.) dropwise with stirring at 25° C. To this was added POCl₃ (1362 g, 17.8 mol, 2.1 eq.) dropwise with stirring at 40° C. The resulting solution was stirred for overnight at 40° C. in an oil bath. The reaction was then quenched by the addition of K3PO4 (2000 g) in H₂O (5 L). The resulting solution was extracted with dichloromethane (3×10 L) and combined organic was dried over anhydrous sodium sulfate. After filtration, the filtrate was concentrated under reduced pressure. This resulted in This resulted (530 g, Crude) of 2-chloro-4,4-dimethylcyclopent-1-ene-1-carbaldehyde as a brown solid. GC-MS (ES, m/z): 158.

Synthesis of 4,4-dimethyl-1,10-diazatricyclo[6.4.0.0\hat{ }[2,6]]dodeca-2(6),7-dien-9-one: Into a 5-L 4-necked round-bottom flask, was placed 2-chloro-4,4-dimethylcyclopent-1-ene-1-carbaldehyde (474.0 g, 2988.1 mmol, 1.0 eq.), N,N-dimethylformamide (3 L), piperazin-2-one (299.2 g, 2988.1 mmol, 1.0 eq.), DIEA (463.4 g, 3585.7 mmol, 1.2 eq.). The resulting solution was stirred for overnight at 115° C. in an oil bath. The reaction mixture was cooled to 25° C. with a water/ice bath . The solids were collected by filtration. The resulting mixture was washed with H₂O (3×2 L) and petroleum ether (3×2 L). The combined organic was dried over anhydrous sodium sulfate. After filtration, the filtrate was concentrated under reduced pressure. This resulted (30.0 g, 37.7%) of 4,4-dimethyl-1,10-diazatricyclo[6.4.0.0\hat{ }[2,6]]dodeca-2(6),7-dien-9-one as a grey solid.LC-MS (ES, m/z) M+1: 205.

Synthesis of 2-(3-bromo-5-fluoro-2-methylphenyl)-7,7-dimethyl-3,4,7,8-tetrahydro-2H-cyclopenta[4,5]pyrrolo[1,2-a]pyrazin-1(6H)-one: Into a 250 mL 3-necked round-bottom flask with N,N- chmethylacetamide (100 mL) were added 1,3-dibromo-5-fluoro-2-methylbenzene (5.0 g, 18.7 mmol, 1.0 eq.) and 7,7-dimethyl-3,4,7,8-tetrahydro-2H-cyclopenta[4,5]pyrrolo[1,2-a]pyrazin-1(6H)-one (4.6 g, 22.4 mmol, 1.2 eq.) at 25° C. To the above mixture was added Cul (2.8 g, 14.9 mmol, 0.8 eq.), K₂CO₃(7.7 g, 56.0 mmol, 3.0 eq.) and 1,10-phenanthroline (2.0 g, 11.2 mmol, 0.6 eq.) at 25° C. The resulting mixture was stirred for 16 hours at 110° C. The reaction mixture was cooled to 25° C. The resulting mixture was diluted with H₂O (200 mL). The resulting mixture was extracted with ethyl acetate (2×200 mL). The combined organic layers were washed with brine (1×200 mL), dried over anhydrous Na₂SO₄. After filtration, the filtrate was concentrated under reduced pressure. The crude residue was purified by silica gel column (silica gel, dichloromethane/ ethyl acetate (=1:1) to afford 2-(3-bromo-5-fluoro-2-methylphenyl)-7,7-dimethyl-3,4,7,8-tetrahydro-2-H-cyclopenta[4,5]pyrrolo[1,2-a]pyrazin-1(6H)-one (1200 mg, 16.4%) as white solid. LC-MS (ESI, m/z) M+1:391.

Synthesis of 2-(5-fluoro-2-methyl-3-(4,4,5,5-tetramethyl-1,3,2-dioxaborolan-2-yl)phenyl)-7,7-dimethyl-3,4,7,8-tetrahydro-2H-cyclopenta[4,5]pyrrolo[1,2-a]pyrazin-1(6H)-one: Into a 100 mL 3-necked round-bottom flask purged and maintained with an inert atmosphere of nitrogen, was placed 2-(3-bromo-5-fluoro-2-methylphenyl)-7,7-dimethyl-3,4,7,8-tetrahydro- 2H -cyclopenta[4,5]pyrrolo[1,2-a]pyrazin-1(6H)-one (1.2 g, 3.1 mmol, 1.0 eq.), bis(pinacolato)diboron (1.6 g, 6.2 mmol, 2.0 eq.), KOAc (903.0 mg, 9.3 mmol, 3.0 eq.), Pd(dppf)Cl₂ (249.8 mg, 0.3 mmol, 0.1 eq.), dioxane (20 mL). The resulting solution was stirred for 6 hours at 100° C. in an oil bath. The reaction was then quenched by the addition of 30 mL of H₂O. The resulting solution was extracted with 3×50 mL of ethyl acetate. The combined organic layers were washed with brine (1×40 mL), dried over anhydrous Na₂SO4. After filtration, the filtrate was concentrated under reduced pressure. The crude residue was purified by a flash column (silica gel, ethyl acetate/petroleum ether=1:3). This resulted in 2-(5-fluoro-2-methyl-3-(4,4,5,5-tetramethyl-1,3,2-dioxaborolan-2-yl)phenyl)-7,7-dimethyl-3,4,7,8-tetrahydro-2H-cyclopenta[4,5]pyrrolo[1,2-a]pyrazin-1(6H)-one (210 mg, 15.6%) as white solid.LC-MS (ESI, m/z) M+1: 439.

Synthesis of 10-[5-fluoro-3-(5-methoxypyrimidin-4-yl)-2-methylphenyl]-4,4-dimethyl-1,10-diazatricyclo[6.4.0.0\hat{ }{2,6}]dodeca-2(6),7-dien-9-one: Into a 40 mL vial were added 4-chloro-5-methoxypyrimidine (150 mg, 1.0 mmol, 1.0 eq.) and 10-[5-fluoro-2-methyl-3-(4,4,5,5-tetramethyl-1,3,2-dioxaborolan-2-yl)phenyl]-4,4-dimethyl-1,10-diazatricyclo[6.4.0.0\hat{ }{2,6}]dodeca-2(6),7-dien-9-one (454.8 mg, 1.0 mmol, 1.0 eq.),K₂CO₃ (286.8 mg, 2.0 mmol, 2.0 eq.), Pd(dppf)Cl₂ (37.9 mg, 0.05 mmol, 0.05 eq.), DME (4 mL), H₂O (0.4 mL) at 25° C. The resulting mixture was stirred for 2 hours at 110° C. under nitrogen atmosphere. The mixture was allowed to cool down to 25° C. The resulting mixture was diluted with H₂O (10mL). The resulting mixture was extracted with ethyl acetate (3×20 mL). The combined organic layers were washed with brine (1×20mL), dried over anhydrous Na₂SO₄. After filtration, the filtrate was concentrated under reduced pressure. The crude residue was purified by a flash column (silica gel, ethyl acetate/petroleum ether=10:1-1:1) to afford 10-[5-fluoro-3-(5-methoxypyrimidin-4-yl)-2-methylphenyl]-4,4-dimethyl-1,10-diazatricyclo[6.4.0.0\hat{ }{2,6}]dodeca-2(6),7-dien-9-one (300 mg, 68.7%) as white solid.LC-MS (ESI, m/z) M+1: 421.

Synthesis of 10-[5-fluoro-3-(5-hydroxypyrimidin-4-yl)-2-methylphenyl]-4,4-dimethyl-1,10-diazatricyclo[6.4.0.0\hat{ }{2,6}]clodeca-2(6),7-dien-9-one: Into a 40 mL round-bottom flask were added 10-[5-fluoro-3-(5-methoxypyrimidin-4-yl)-2-methylphenyl]-4,4-dimethyl-1,10-diazatricyclo[6.4.0.0\hat{ }{2,6}]dodeca-2(6),7-dien-9-one (300 mg, 0.7 mmol, 1.0 eq.) and CH₂Cl₂ (1 mL), 1 M BBr3 in CH₂Cl₂ (2.8 mL) at 25° C. The resulting mixture was stirred for 12 hours at 40° C. The mixture was allowed to cool down to 25° C. The reaction was quenched by the addition of MeOH (3mL) at 25° C. The resulting mixture was concentrated under reduced pressure. The crude residue was purified by a flash column (silica gel, ethyl acetate/petroleum ether=1:1) to afford 10-[5-fluoro-3-(5-hydroxypyrimidin-4-yl)-2-methylphenyl]-4,4-dimethyl-1,10-diazatricyclo[6.4.0.0\hat{ }{2,6}]dodeca-2(6),7-dien-9-one (200 mg, 68.9%) as white solid. LC-MS (ESI, m/z) M+1:407.

Synthesis of tert-butyl (2S)-2-({[4-(3-[4,4-dimethyl-9-oxo-1,10-diazatricyclo [6.4.0.0\hat{ }{2,6}]dodeca-2(6),7-dien-10-yl]-5-fluoro-2-methylphenyl)pyrimidin-5 -yl]oxy}methyl)pyrrolidine-1-carboxylate: Into a 50 mL 3-necked round-bottom flask were added 10-[5-fluoro-3-(5-hydroxypyrimidin-4-yl)-2-methylphenyl]-4,4-dimethyl-1,10-diazatricyclo[6.4.0.0\hat{ }{2,6}]dodeca-2(6),7-dien-9-one (190 mg, 0.4 mmol, 1.0 eq.) and tert-butyl (2S)-2-(hydroxymethyl)pyrrolidine-1-carboxylate (188.1 mg, 0.9 mmol, 2.0 eq.), PPh₃ (245.2 mg, 0.9 mmol, 2.0 eq.), tetrahydrofuran (3 mL) at 0° C. To the above mixture was added DIAD (189.0 mg, 0.9 mmol, 2.0 eq.) dropwise at 0° C. The resulting mixture was stirred for additional 6 hours at 0° C. The resulting mixture was diluted with H₂O (10 mL). The resulting mixture was extracted with ethyl acetate (3×10 mL). The combined organic layers were washed with brine (1×10mL), dried over anhydrous Na₂SO₄. After filtration, the filtrate was concentrated under reduced pressure. The crude residue was purified by a flash column (silica gel, ethyl acetate/petroleum ether=10:1-1:1) to afford tert-butyl (2S)-2-({[4-(3-{4,4-dimethyl-9-oxo-1,10-diazatricyclo[6.4.0.0\hat{ }{2,6}]dodeca-2(6),7-dien-10-yl}-5-fluoro-2-methylphenyl)pyrimidin-5-yl]oxy }methyl)pyrrolidine- 1 -carboxylate (80 mg, 29.0%) as white solid. LC-MS (ESI, m/z) M+1: 590.

Synthesis of 10-(5-fluoro-2-methyl-3-{5-[(2S)-pyrrolidin-2-ylmethoxy]pyrimidin-4-yl}phenyl)-4,4-dimethyl-1,10-diazatricyclo[6.4.0.0\hat{ }{2,6}]dodeca-2(6),7-dien-9-one: Into a 50 mL round-bottom flask were added tert-butyl (2S)-2-({[4-(3-{4,4-dimethyl-9-oxo-1,10-diazatricyclo[6.4.0.0\hat{ }{2,6}]dodeca-2(6),7-dien-10-yl}-5-fluoro-2-methylphenyl)pyrimidin-5-yl]oxy}methyl)pyrrolidine-1-carboxylate (80 mg, 0.1 mmol, 1.0 eq.) and 2M HCl (gas) in ethyl acetate (2 mL) at 25° C. The resulting mixture was stirred for 3 hours at 25° C. The resulting mixture was concentrated under reduced pressure. This resulted in 10-(5-fluoro-2-methyl-3-{5-1(2S)-pyrrolidin-2-ylmethoxylpyrimidin-4-yl}phenyl)-4,4-dimethyl-1,10-diazatricyclo[6.4.0.0\hat{ }{2,6}]dodeca-2(6),7-dien-9-one (60 mg, 90.3%) as white solid.LC-MS (ESI, m/z) M+1: 490.

Synthesis of 10-[5-fluoro-2-methyl-3-(5-{[(2S)-1-(prop-2-enoyl)pyrrolidin-2-yl]methoxy}pyrimidin-4-yl)phenyl1-4,4-dimethyl-1,10-diazatricyclo[6.4.0.0\hat{ }{2,6}]dodeca-2(6),7-dien-9-one: Into a 8 mL vial were added 10-(5-fluoro-2-methyl-3-{ 5- R2S)-pyrrolidin-2-ylmethoxylpyrimidin-4-yl}phenyl)-4,4-dimethyl-1,10-diazatricyclo[6.4.0.0\hat{ }{2,6}]dodeca-2(6),7-dien-9-one (60 mg, 0.1 mmol, 1.0 eq.), CH₂Cl₂ (1 mL) and trimethylamine (24.8 mg, 0.2 mmol, 2.0 eq.) at 0° C. To the above mixture was added acryloyl chloride (11.1 mg, 0.1 mmol, 1.0 eq.) dropwise at 0° C. The resulting mixture was stirred for additional 1 hour at 0° C. The resulting mixture was concentrated. The crude product was purified by Chiral-Prep-HPLC with the following conditions (Prep-HPLC-003): Column, SunFire Prep C₁₈ OBD Column, 19*150 mm, 5 μm 1,10nm; mobile phase, H₂(0.05% TFA) and CH3CN (30% CH₃CN up to 68% in 7 min); Detector, UV 220 to afford 10-15-fluoro-2-methyl-3-(5-{[(2S)-1-(prop-2-enoyl)pyrrolidin-2-yl]methoxy}pyrimidin-4-yl)phenyl]-4,4-dimethyl-1,10-diazatricyclo[6.4.0.0\hat{ }{2,6}]dodeca-2(6),7-dien-9-one (8 mg, 12.1%) as white solid. LC-MS (ESI, m/z) M+1:544. 1 HNMR (300 MHz, DMSO-d₆)45 8.90 (s, 1H), 8.75 (s, 1H), 7.28 (dd, J=9.0, 2.8 Hz, 1H), 7.20-7.02 (m, 1H), 6.70-6.45 (m, 2H), 6.29-6.14 (m, J=16.8, 9.0, 2.0 Hz, 1H), 5.77-5.58 (m, 1H), 4.61-4.05 (m, 6H), 4.01-3.73 (m, 1H), 3.71-3.37 (m, 2H), 2.73-2.39 (m, 4H), 2.18-1.68 (m, 7H), 1.27 (s, 6H).

Example A: The compounds below are prepared by methods substantially identical, similar, or analogous to those disclosed in the General Scheme and above Examples.

| ID | Chemical Structure | m/z[MH+] |
|---|---|---|
| A-2 | (S)-N-(4-(6-((3-acrylamido-4-(2-methyl-4-(tetrahydro-2H- | 753 |
| | pyran-4-yl)piperazin-1-yl)phenyl)amino)-4-methyl-5-oxo- | |
| | 4,5-dihydropyrazin-2-yl)-3-(hydroxymethyl)pyridin-2-yl)-4- | |
| | (tert-butyl)-2-fluorobenzamide, | |
| A-3 | (S)-N-(4-(6-((3-acrylamido-4-(2-methyl-4-(tetrahydro-2H-pyran- | 737 |
| | 4-yl)piperazin-1-yl)phenyl)amino)-4-methyl-5-oxo-4,5- | |
| | dihydropyrazin-2-yl)-3-(hydroxymethyl)pyridin-2-yl)-4- | |
| | cyclopropyl-2-fluorobenzamide, | |
| A-4 | (S)-N-(4-(6-((3-acrylamido-4-(2-methyl-4-(tetrahydro-2H- | 719 |
| | pyran-4-yl)piperazin-1-yl)phenyl)amino)-4-methyl-5-oxo- | |
| | 4,5-dihydropyrazin-2-yl)-3-(hydroxymethyl)pyridin-2-yl)-4- | |
| | cyclopropylbenzamide, | |
| A-5 | (S)-N-(4-(6-((3-acrylamido-4-(2-methyl-4-(tetrahydro-2H-pyran- | 750 |
| | 4-yl)piperazin-1-yl)phenyl)amino)-4-methyl-5-oxo-4,5- | |
| | dihydropyrazin-2-yl)-3-(hydroxymethyl)pyridin-2-yl)-1,5,5- | |
| | trimethyl-1,4,5,6-tetrahydrocyclopenta[b]pyrrole-2-carboxamide, | |
| A-6 | (S)-N-(4-(6-((3-acrylamido-4-(2-methyl-4-(tetrahydro-2H-pyran- | 736 |
| | 4-yl)piperazin-1-yl)phenyl)amino)-4-methyl-5-oxo-4,5- | |
| | dihydropyrazin-2-yl)-3-(hydroxymethyl)pyridin-2-yl)-5,5- | |
| | dimethyl-1,4,5,6-tetrahydrocyclopenta[b]pyrrole-2-carboxamide, | |
| A-7 | (S)-N-(4-(6-((3-acrylamido-4-(2-methyl-4-(tetrahydro-2H-pyran- | 741 |
| | 4-yl)piperazin-1-yl)phenyl)amino)-4-methyl-5-oxo-4,5- | |
| | dihydropyrazin-2-yl)-3-(hydroxymethyl)pyridin-2-yl)-5-(tert- | |
| | butyl)thiophene-2-carboxamide, | |
| A-8 | (S)-N-(4-(6-((3-acrylamido-4-(2-methyl-4-(tetrahydro-2H-pyran- | 739 |
| | 4-yl)piperazin-1-yl)phenyl)amino)-4-methyl-5-oxo-4,5- | |
| | dihydropyrazin-2-yl)-3-(hydroxymethyl)pyridin-2-yl)-4,5,6,7- | |
| | tetrahydrobenzo[b]thiophene-2-carboxamide, | |
| A-9 | (S)-N-(6-((3-acrylamido-4-(2-methyl-4-(tetrahydro-2H-pyran-4- | 722 |
| | yl)piperazin-1-yl)phenyl)amino)-3'-(hydroxymethyl)-[2,4'- | |
| | bipyridin]-2'-yl)-4-(tert-butyl)-2-fluorobenzamide, | |
| A-10 | (S)-N-(4-(6-((3-acrylamido-4-(2-methyl-4-(tetrahydro-2H-pyran- | 723 |
| | 4-yl)piperazin-1-yl)phenyl)amino)pyrazin-2-yl)-3- | |
| | (hydroxymethyl)pyridin-2-yl)-4-(tert-butyl)-2-fluorobenzamide, | |
| A-11 | (S)-N-(4-(2-((3-acrylamido-4-(2-methyl-4-(tetrahydro-2H-pyran- | 707 |
| | 4-yl)piperazin-1-yl)phenyl)amino)pyrimidin-4-yl)-3- | |
| | (hydroxymethyl)pyridin-2-yl)-4-cyclopropyl-2-fluorobenzamide, | |
| A-12 | (S)-N-(4-(4-((3-acrylamido-4-(2-methyl-4-(tetrahydro-2H-pyran- | 708 |
| | 4-yl)piperazin-1-yl)phenyl)amino)-1,3,5-triazin-2-yl)-3- | |
| | (hydroxymethyl)pyridin-2-yl)-4-cyclopropyl-2-fluorobenzamide, | |
| A-13 | (S)-N-(4-(4-((3-acrylamido-4-(2-methyl-4-(tetrahydro-2H-pyran- | 723 |
| | 4-yl)piperazin-1-yl)phenyl)amino)pyrimidin-2-yl)-3- | |
| | (hydroxymethyl)pyridin-2-yl)-4-(tert-butyl)-2-fluorobenzamide, | |
| A-14 | (S)-N-(4-(4-((3-acrylamido-4-(2-methyl-4-(tetrahydro-2H-pyran- | 791 |
| | 4-yl)piperazin-1-yl)phenyl)amino)-5-(trifluoromethyl)pyrimidin-2- | |
| | yl)-3-(hydroxymethyl)pyridin-2-yl)-4-(tert-butyl)-2- | |
| | fluorobenzamide, | |
| A-15 | (S)-N-(4-(4-((3-acrylamido-4-(2-methyl-4-(tetrahydro-2H-pyran- | 753 |
| | 4-yl)piperazin-1-yl)phenyl)amino)-5-methoxypyrimidin-2-yl)-3- | |
| | (hydroxymethyl)pyridin-2-yl)-4-(tert-butyl)-2-fluorobenzamide, | |
| A-16 | (S)-N-(4-(4-((3-acrylamido-4-(2-methyl-4-(tetrahydro-2H-pyran- | 781 |
| | 4-yl)piperazin-1-yl)phenyl)amino)-5-isopropoxypyrimidin-2-yl)-3- | |
| | (hydroxymethyl)pyridin-2-yl)-4-(tert-butyl)-2-fluorobenzamide, | |
| A-17 | (S)-N-(4-(4-((3-acrylamido-4-(2-methyl-4-(tetrahydro-2H-pyran- | 738 |
| | 4-yl)piperazin-1-yl)phenyl)amino)-5-aminopyrimidin-2-yl)-3- | |
| | (hydroxymethyl)pyridin-2-yl)-4-(tert-butyl)-2-fluorobenzamide, | |
| A-18 | (S)-N-(4-(4-((3-acrylamido-4-(2-methyl-4-(tetrahydro-2H-pyran- | 752 |
| | 4-yl)piperazin-1-yl)phenyl)amino)-5-(methylamino)pyrimidin-2- | |
| | yl)-3-(hydroxymethyl)pyridin-2-yl)-4-(tert-butyl)-2- | |
| | fluorobenzamide, | |
| A-19 | (S)-3-((3-acrylamido-4-(2-methyl-4-(tetrahydro-2H-pyran-4- | 738 |
| | yl)piperazin-1-yl)phenyl)amino)-1-(2-(4-cyclopropyl-2- | |
| | fluorobenzamido)-3-(hydroxymethyl)pyridin-4-yl)-1H- | |
| | pyrazole-4-carboxamide, | |
| A-20 | (S)-N-(4-(3-((3-acrylamido-4-(2-methyl-4-(tetrahydro-2H- | 764 |
| | pyran-4-yl)piperazin-1-yl)phenyl)amino)-4-oxo-4,5,6,7- | |
| | tetrahydro-1H-pyrazolo[4,3-c]pyridin-1-yl)-3- | |
| | (hydroxymethyl)pyridin-2-yl)-4-cyclopropyl-2- | |
| | fluorobenzamide, | |
| A-21 | (S)-N-(4-(8-((3-acrylamido-4-(2-methyl-4-(tetrahydro-2H-pyran- | 762 |
| | 4-yl)piperazin-1-yl)phenyl)amino)imidazo[1,2-a]pyrazin-6-yl)-3- | |
| | (hydroxymethyl)pyridin-2-yl)-4-(tert-butyl)-2-fluorobenzamide, | |
| A-22 | (S)-N-(4-(6-((3-acrylamido-4-(2-methyl-4-(oxetan-3-yl)piperazin- | 709 |
| | 1-yl)phenyl)amino)-4-methyl-5-oxo-4,5-dihydropyrazin-2-yl)-3- | |
| | (hydroxymethyl)pyridin-2-yl)-4-cyclopropyl-2-fluorobenzamide, | |
| A-23 | (R)-N-(4-(6-((3-acrylamido-4-(4-(oxetan-3-yl)-2- | 763 |
| | (trifluoromethyl)piperazin-1-yl)phenyl)amino)-4-methyl-5-oxo- | |
| | 4,5-dihydropyrazin-2-yl)-3-(hydroxymethyl)pyridin-2-yl)-4- | |
| | cyclopropyl-2-fluorobenzamide, | |
| A-24 | (S)-N-(4-(6-((3-acrylamido-4-(2-methyl-4-(tetrahydro-2H-pyran- | 721 |
| | 4-yl)piperazin-1-yl)phenyl)amino)-4-methyl-5-oxo-4,5- | |
| | dihydropyrazin-2-yl)-3-methylpyridin-2-yl)-4-cyclopropyl-2- | |
| | fluorobenzamide. | |

Example B: The compounds below are prepared by methods substantially identical, similar, or analogous to those disclosed in the General Scheme and above Examples.

| ID | Chemical Structure | m/z[MH+] |
|---|---|---|
| B-1 | (S,E)-2-(3-(5-(2-(1-acryloylpyrrolidin-2-yl)vinyl)-6- | 537 |
| | aminopyrimidin-4-yl)-2-methylphenyl)-7,7-dimethyl-3,4,7,8- | |
| | tetrahydro-2H-cyclopenta[4,5]pyrrolo[1,2-a]pyrazin-1(6H)- | |
| | one | |
| B-2 | (S,E)-2-(3-(3-(2-(1-acryloylpyrrolidin-2-yl)vinyl)-2- | 536 |
| | aminopyridin-4-yl)-2-methylphenyl)-7,7-dimethyl-3,4,7,8- | |
| | tetrahydro-2H-cyclopenta[4,5]pyrrolo[1,2-a]pyrazin-1(6H)- | |
| | one | |
| B-3 | (S,E)-2-(3'-(2-(1-acryloylpyrrolidin-2-yl)vinyl)-2'-amino-3- | 537 |
| | methyl-[4,4'-bipyridin]-2-yl)-7,7-dimethyl-3,4,7,8-tetrahydro- | |
| | 2H-cyclopenta[4,5]pyrrolo[1,2-a]pyrazin-1(6H)-one | |

Example B: The compounds below are prepared by methods substantially identical, similar, or analogous to those disclosed in the General Scheme and above Examples.

| ID | Chemical Structure | m/z[MH+] |
|---|---|---|
| B-4 | (S)-2-(4-(5-((1-acryloylpyrrolidin-2-yl)methoxy)-6- | 558 |
| | aminopyrimidin-4-yl)-3-(hydroxymethyl)pyridin-2-yl)-7,7- | |
| | dimethyl-3,4,7,8-tetrahydro-2H-cyclopenta[4,5]pyrrolo[1,2- | |
| | alpyrazin-1(6H)-one, | |

### Biological Example 1: Binding Constant (K_{d}) Determination

The K_{d} of the compounds were determined by KINOMEscan™ assay, the industry's most comprehensive high-throughput system for screening compounds against large numbers of human kinases. KINOMEscan™ assay is based on a competition binding assay that quantitatively measures the ability of a compound to compete with an immobilized, active-site directed ligand. The assay is performed by combining three components: DNA-tagged kinase; immobilized ligand; and a test compound. The ability of the test compound to compete with the immobilized ligand is measured via quantitative PCR of the DNA tag. The kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in lx binding buffer (20% SeaBlock, 0.17×PBS, Tween 20, 6 mM DTT). All reactions were performed in polystyrene 96-well plates in a final volume of 0.135 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, nonbiotinylated affinity ligand) and incubated at room temperature with shaking for minutes. The kinase concentration in the eluates was measured by qPCR. An 11-point 3-fold serial dilution of each test compound was prepared in 100% DMSO at 100× final test concentration and subsequently diluted to 1× in the assay (final DMSO concentration=1%). Most K_{d} were determined using a compound top concentration=30,000 nM. If the initial K_{d} determined was <0.5 nM (the lowest concentration tested), the measurement was repeated with a serial dilution starting at a lower top concentration. A K_{d} value reported as 40,000 nM indicates that the K_{d} was determined to be >30,000 nM. Binding constants (K_{d}s) were calculated with a standard dose-response curve using the Hill equation: Response=Background+(Signal−Background)/[1+(K_{d}^{Hill Slope}/Dose^{Hill Slope})]. The Hill Slope was set to −1. Curves were fitted using a non-linear least square fit with the Levenberg-Marquardt algorithm. Such assays, carried out with a range of doses of test compounds, allow the determination of an approximate K_{d} value. Although the K_{d} of the compounds of the present invention vary with structural change as expected, the activity generally exhibited by these agents is in the range of K_{d}=0.1-1000 nM.

### Biological Example 2: In Vitro Dialysis Assay (Arreversibility Assay)

IC50 of the test compound, was determined in the presence of 0.1 nM Enzyme and 40 mM ATP. 0.003 mM Compound (39xIC50 @40 mM ATP) was pre-incubated with 2 nM BTK for 2hr in assay buffer without ATP. The compound-enzyme complex was dialyzed against the same buffer supplemented with 40 mM ATP for 24 hr. Cumulative dialysis factor >160,000×. After dialysis, the BTK activity was measured in the presence 40 mM ATP and 1 mM substrate peptide and compared to that in non-dialyzed samples. Assay buffer: 100 mM HEPES, pH7.5; 0.1% BSA, 0.01% Triton-X 100; 5 mM MgCl₂; 1 mM DTT. In this study, Ibrutinib (a FDA approved irreversible BTK inhibitor) was used as a positive control and Saurosporine (a reversible BTK inhibitor) was used as a negative control. The following table shows the recovery after 24 hr dialysis.

In this assay, GDC-0853, a reversible BTK inhibitor, was used as a reference compound. GDC-0853, disclosure in WO 2013067274, is an orally bioavailable, selective, and reversible Bruton's tyrosine kinase (BTK) inhibitor with IC50s ranging from 2-9 nM for basophil activation, B cell receptor activation, and constitutive p-BTK activity in whole blood lysates. 1,2 In rats, treatment for longer than 7 days leads to pancreatic toxicity but it does not occur in mice or dogs, even at higher doses. Formulations containing GDC-0853 were well-tolerated in Phase I clinical trials and are in additional clinical trials for rheumatoid arthritis, lupus erythematosus and other autoimmune diseases.

### Biological Example 3: Biochemical Enzymatic Assay (IC50) Against WT BTK

A Caliper-based kinase assay (Caliper Life Sciences, Hopkinton, MA) was used to measure inhibition of WT Btk kinase activity of a compound of the present disclosure. Ibrutinib and ACP=196 was used as control compounds. Serial dilutions of test compounds were incubated with human recombinant WT BTK (0.5 nM), ATP (16 μM) and a phosphoacceptor peptide substrate FAM-GEEPLYWSFPAKKK-NH2 (1 μM) at room temperature for 3 h. The reaction was then terminated with EDTA, final concentration 20 mM and the phosphorylated reaction product was quantified on a Caliper Desktop Profiler (Caliper LabChip 3000). Percent inhibition was calculated for each compound dilution and the concentration that produced 50% inhibition was calculated.

The following table shows the IC50 of WT BTK for Ibrunitib, ACP-196, certain compounds of the disclosure.

| Covalent BTKi | WT BTK IC50 (nM) |
|---|---|
| Acalabrutinib(ACP-196) | 7.0 |
| Example 1 | 0.64 |

### Biological Example 5: In Vitro BTK Active Site Occupancy Assays in REC-1 Cells

The time-resolved fluorescent energy transfer technology (TR-FRET/HTRF) to set up the BTK active site occupancy assays. The assays will utilize the following key components: Ramos B cells, maintained in RPMI 1640 media supplemented with 10% FBS and pen/strep antibiotics under 5% CO₂. Biotinylated probe: Biotin-Ibrutinib (MedChemExpress). HTRF Detection antibodies: Streptavidin-d2 and Eu+-cryptate-labeled anti BTK antibody (CisBio). Assay will be developed in 96 well plates in HTRF format. To determine percent of the BTK's active site occupancy, the cells in growth media will be first treated with test compound (or 0.1% DMSO) for 1 h at 37° followed by incubation with biotinylated derivative of ibrutinib (covalent BTK inhibitor) for another 1 hr. Next, the cells will be lysed in a buffer provided with the total BTK HTRF detection kit (CisBio). The biotinylated Ibrutinib bound to a free pool of the BTK protein will be detected by HTRF using streptavidin-d2 and Eu+-cryptate-labeled anti BTK antibody (CisBio). HTRF signal (due to proximity between biotin- ibrutinib and BTK) will be measured and compared to that in the untreated control sample to calculate %-occupancy by the test compound. All samples will be tested in triplicate wells.

### Biological Example 6: Inhibition of CD69 Surface Expression in Primary Human B Cells (Western Analysis)

Primary human B cells (CD20+, purified by negative selection) were obtained from StemCell Technologies. Prior to experiment, the cells were thawed and washed two times with RPMI growth media supplemented with 10% FBS. The cells were seeded into 24 well plate at a density of 4×105 cells/per a well in a total volume of 500 uL. 6 hr after plating, serial dilutions of NW-1-96 were added. Control wells received DMSO only (0.1%). After lh pre-incubation with compound, the cells were stimulated for 19hr with goat anti-human IgM F(ab′)2 (10 μg/mL; ThermoFisher). After stimulation, the cells were fixed by addition of paraformaldehyde to a final concentration of 4% and incubated for 20 min at room temperature. Fixed cells were collected into Eppendorf tubes, centrifuged at 1,000×g and washed three times with 50 mM Tris pH8.0, 100 mM NaCl. After washes, the cells were re-suspended in 100 uL of 50 mM Tris pH8.0, 100 mM NaCl, 0.1% BSA supplemented with 5 ug/mL of FITC-conjugated anti-CD69 antibody (ThermoFisher) and incubated at room temperature for 2hr. The cells were next washed 3 times with 10 volumes of 50 mM Tris pH8.0, 100 mM NaCl, 0.1% BSA and re-suspended in 150 ul of the same buffer. Stained cells were transferred into black 96well plate (100 uL suspension per well) and allowed to sediment for lhr. CD69 staining was detected on Synergy Neo2 fluorescent plate reader: 485 nm emission, 528 nm excitation.

### Biological Example 7: HepG2 in Vitro Liver Toxicity Assay

Cell antiproliferation is assayed by PerkinElmer ATPlite™ Luminescence Assay System. Briefly, the liver cancer cell line HepG2 are plated at a density of about 1×10⁴ cells per well in Costar 96-well plates, and are incubated with different concentrations of compounds for about 72 hours in medium supplemented with 5% FBS. One lyophilized substrate solution vial is then reconstituted by adding 5 mL of substrate buffer solution, and is agitated gently until the solution is homogeneous. About 50 μL of mammalian cell lysis solution is added to 100 μL of cell suspension per well of a microplate, and the plate is shaken for about five minutes in an orbital shaker at −700 rpm. This procedure is used to lyse the cells and to stabilize the ATP. Next, 50 μL substrate solution is added to the wells and microplate is shaken for five minutes in an orbital shaker at −700 rpm. Finally, the luminescence is measured by a PerkinElmer TopCount® Microplate Scintillation Counter. Such assays, carried out with a range of doses of test compounds, allow the determination of the cellular anti-antiproliferative IC₅₀ of the compounds of the present invention.

### Biological Example 8: Human Primary Hepatocyte Cytotoxicity

Cell viability assay is assayed by PerkinElmer ATPliteTM Luminescence Assay System. Briefly, the human primary hepatocyte are plated at a density of about 1×10⁴ cells per well in Costar 96-well plates, and are incubated with different concentrations of compounds for about 72 hours in medium supplemented with 5% FBS. One lyophilized substrate solution vial is then reconstituted by adding 5 mL of substrate buffer solution, and is agitated gently until the solution is homogeneous. About 50 μL of mammalian cell lysis solution is added to 100 μL of cell suspension per well of a microplate, and the plate is shaken for about five minutes in an orbital shaker at −700 rpm. This procedure is used to lyse the cells and to stabilize the ATP. Next, 50 μL substrate solution is added to the wells and microplate is shaken for five minutes in an orbital shaker at −700 rpm. Finally, the luminescence is measured by a PerkinElmer TopCount® Microplate Scintillation Counter. Such assays, carried out with a range of doses of test compounds, allow the determination of the cellular anti-antiproliferative IC₅₀ of the compounds of the present invention.

### Biological Example 9: Mice PK Study

The pharmacokinetics of compounds were evaluated in CD-1 mouse via Intravenous and Oral Administration. The IV dose was administered as a slow bolus in the Jugular vein, and oral doses were administered by gavage. The fomulaltion for IV dosing is 5% DMSO in 20% HPBCD in water, and the PO formulation is 2.5% DMSO, 10% EtOH, 20% Cremphor EL, 67.5% D5W. The PK time point for the IV arm was 5, 15, 30 min, 1, 2, 4, 6, 8, 12, 24 hours post dose, and for PO arm was 15, 30 min, 1, 2, 4, 6, 8, 12, 24 hours post dose. Approximately 0.03 mL blood was collected at each time point. Blood of each sample was transferred into plastic micro centrifuge tubes containing EDTA-K2 and plasma was collected within 15 min by centrifugation at 4000 g for 5 minutes in a 4° C. centrifuge. Plasma samples were stored in polypropylene tubes. The samples were stored in a freezer at -prior to analysis. Concentrations of compounds in the plasma samples were analyzed using a LC-MS/MS method. WinNonlin (Phoenix™, version 6.1) or other similar software was used for pharmacokinetic calculations. The following pharmacokinetic parameters were calculated, whenever possible from the plasma concentration versus time data: IV administration: C₀, CL, V_{d}, T_{1/2}, AUC_{inf}, AUCₗₐₛₜ, MRT, Number of Points for Regression; PO administration: Cₘₐₓ, Tₘₐₓ, T_{1/2}, AUC_{inf}, AUCₗₐₛₜ, F %, Number of Points for Regression. The pharmacokinetic data was described using descriptive statistics such as mean, standard deviation. Additional pharmacokinetic or statistical analysis was performed at the discretion of the contributing scientist, and was documented in the data summary. The PK results of oral dosing of po, 10 mg/kg of certain compounds is shown in the Table below. For comparison purpose, the PK results of oral dosing of po, 25 mg/kg of Acalbrutinib, a FDA approved BTK inhibitor, were obtained using the same methods.

### Biological Example A: Calcium Flux Fluoresence-Based Assay

Calcium flux fluoresence-based assays were performed in a FlexStation 11384 fluorometric imaging plate reader (Molecular Devices) according to manufacturer instructions. In brief, actively growing Ramos cells (ATCC) in RPM1 medium supplemented with 10% FBS (Invitrogen) were washed and re-plated in low serum medium at approximately 5×10⁵ cells per 100 μl per well in a 96-well plate. Compounds to be assayed were dissolved in DMSO and then diluted in low serum medium to final concentrations ranging from 0 to 10 μM (at a dilution factor of 0.3). The diluted compounds were then added to each well (final DMSO concentration was 0.01%) and incubated at 37 degree in 5% CO₂ incubator for one hour. Afterwards, 100 μl of a calcium-sensitive dye (from the Calcium 3 assay kit, Molecular Devices) was added to each well and incubated for an additional hour. The compound-treated cells were stimulated with a goat anti-human IgM antibody (80 ug/ml; Jackson ImmunoResearch) and read in the FlexStation 11384 using a λ_{Ex}=485 nm and λ_{Em}=538 nm for 200 seconds. The relative fluorescence unit (RFU) and the IC₅₀ were recorded and analyzed using a built-in SoftMax program (Molecular devices).

Biological Example B: Inhibition of B-cell Activation—B cell FLIPR Assay in Ramos Cells

Inhibition of B-cell activation by compounds of the present invention is demonstrated by determining the effect of the test compounds on anti-IgM stimulated B cell responses. The B cell FLIPR assay is a cell based functional method of determining the effect of potential inhibitors of the intracellular calcium increase from stimulation by an anti-IgM antibody. Ramos cells (human Burkitt's lymphoma cell line. ATCC-No. CRL-1596) were cultivated in Growth Media (described below). One day prior to assay, Ramos cells were resuspended in fresh growth media (same as above) and set at a concentration of 0.5×10⁶/mL in tissue culture flasks. On day of assay, cells are counted and set at a concentration of 1×10⁶/mLl in growth media supplemented with IμM FLUO-3AM(TefLabs Cat-No. 0116, prepared in anhydrous DMSO and 10% Pluronic acid) in a tissue culture flask, and incubated at 37° C. (5% CO₂) for one h. To remove extracellular dye, cells were collected by centrifugation (5 min, 1000 rpm), resuspended in FLIPR buffer (described below) at 1×10⁶ cells/mL and then dispensed into 96-well poly-D-lysine coated black/clear plates (BD Cat-No. 356692) at 1×10⁵ cells per well. Test compounds were added at various concentrations ranging from 100 μM to 0.03 μM (7 concentrations, details below), and allowed to incubate with cells for min at RT. Ramos cell Ca²⁺ signaling was stimulated by the addition of 10 μg/mL anti-IgM (Southern Biotech, Cat-No. 2020-01) and measured on a FLIPR (Molecular Devices, captures images of 96 well plates using a CCD camera with an argon laser at 480 nM excitation).
Growth Medium: RPMI 1640 medium with L-glutamine (Invitrogen, Cat-No. 61870-010), 10% Fetal Bovine Serum (FBS, Summit Biotechnology Cat-No. FP-100-05); ImM Sodium Pyruvate (Invitrogen Cat. No. 11360-070).
FLIPR buffer: HBSS (Invitrogen, Cat-No. 141175-079), 2 mM CaCl₂ (Sigma Cat-No. C-4901), HEPES (Invitrogen, Cat-No. 15630-080), 2.5 mM Probenecid (Sigma, Cat-No. P-8761), 0.1% BSA (Sigma, Cat-No.A-7906), 11 mM Glucose (Sigma, Cat-No.G-7528);
Assay and Analysis: Intracellular increases in calcium were reported using a max -min statistic (subtracting the resting baseline from the peak caused by addition of the stimulatory antibody using a Molecular Devices FLIPR control and statistic exporting software. The IC₅₀ was determined using a nonlinear curve fit (GraphPad Prism).

### Biological Example C: In Vivo Xenograft Studies

Typically, athymic nude mice (CD-1 nu/nu) or SCID mice are obtained at age 6-8 weeks from vendors and acclimated for a minimum 7-day period. The cancer cells are then implanted into the nude mice. Depending on the specific tumor type, tumors are typically detectable about two weeks following implantation. When tumor sizes reach ˜100-200 mm³, the animals with appreciable tumor size and shape are randomly assigned into groups of 8 mice each, including one vehicle control group and treatment groups. Dosing varies depending on the purpose and length of each study, which typically proceeds for about 3-4 weeks. Tumor sizes and body weight are typically measured three times per week. In addition to the determination of tumor size changes, the last tumor measurement is used to generate the tumor size change ratio (T/C value), a standard metric developed by the National Cancer Institute for xenograft tumor evaluation. In most cases, % T/C values are calculated using the following formula: % T/C=100×ΔT/ΔC if ΔT>0. When tumor regression occurred (ΔT<0), however, the following formula is used: % T/TO=100×ΔT/T0. Values of <42% are considered significant.

Biological Example D: Mouse Collagen-Induced Arthritis (mCIA)

On day 0 mice are injected at the base of the tail or several spots on the back with an emulsion of Type II Collagen (i.d.) in Complete Freund's adjuvant (CFA). Following collagen immunization, animals will develop arthritis at around 21 to 35 days. The onset of arthritis is synchronized (boosted) by systemic administration of collagen in Incomplete Freund's adjuvant (IFA; i.d.) at day 21. Animals are examined daily after day 20 for any onset of mild arthritis (score of 1 or 2; see score description below) which is the signal to boost. Following boost, mice are scored and dosed with candidate therapeutic agents for the prescribed time (typically 2-3 weeks) and dosing frequency, daily (QD) or twice-daily (BID). The developing inflammation of the paws and limb joints is quantified using a scoring system that involves the assessment of the 4 paws following the criteria described below:

Scoring:
1=swelling and/or redness of paw or one digit.
2=swelling in two or more joints.
3=gross swelling of the paw with more than two joints involved.
4=severe arthritis of the entire paw and digits.

Evaluations are made on day 0 for baseline measurement and starting again at the first signs or swelling for up to three times per week until the end of the experiment. The arthritic index for each mouse is obtained by adding the four scores of the individual paws, giving a maximum score of 16 per animal.

Biological Example E: Rat Collagen-Induced Arthritis (rCIA)

On day 0, rats are injected with an emulsion of Bovine Type II Collagen in Incomplete Freund's adjuvant (IFA) is injected intradermally (i.d.) on several locations on the back. A booster injection of collagen emulsion is given around day 7, (i.d.) at the base of the tail or alternative sites on the back. Arthritis is generally observed 12-14 days after the initial collagen injection. Animals may be evaluated for the development of arthritis as described below (Evaluation of arthritis) from day 14 onwards. Animals are dosed with candidate therapeutic agents in a preventive fashion starting at the time of secondary challenge and for the prescribed time (typically 2-3 weeks) and dosing frequency, daily (QD) or twice-daily (BID). The developing inflammation of the paws and limb joints is quantified using a scoring system that involves the assessment of the 4 paws following the criteria as described above. Evalulation are made on day 0 for baseline measurement and starting again at the first signs or swelling for up to three times per week until the end of the experiment. The arthritic index for each mouse is obtained by adding the four scores of the individual paws, giving a maximum score of 16 per animal.

### Biological Example F: Rat In Vivo Asthma Model

Male Brown-Norway rats are sensitized i.p. with 100 μg of OA (ovalbumin) in 0.2 ml alum once every week for three weeks (day 0, 7, and 14). On day 21 (one week following last sensitization), the rats are dosed q.d. with either vehicle or compound formulation subcutaneously 0.5 hour before OA aerosol challenge (1% OA for 45 minutes) and terminated 4 or 24 hours after challenge. At time of sacrifice, serum and plasma are collected from all animals for serology and PK, respectively. A tracheal cannula is inserted and the lungs are lavaged 3× with PBS. The BAL fluid is analyzed for total leukocyte number and \hat{ }differential leukocyte counts. Total leukocyte number in an aliquot of the cells (20-100 μ [symbol] ) is determined by Coulter Counter. For differential leukocyte counts, 50-200 μ [symbol] of the sample is centrifuged in a Cytospin and the slide stained with Diff-Quik. The proportions of monocytes, eosinophils, neutrophils and lymphocytes are counted under light microscopy using standard morphological criteria and expressed as a percentage. Representative inhibitors of Btk show decreased total leucocyte count in the BAL of OA sensitized and challenged rats as compared to control levels.

What is claimed is:

1. A compound of Formula (I), or an N-oxide thereof, or a pharmaceutically acceptable salt, solvate, polymorph, tautomer, stereoisomer, an isotopic form, or a prodrug of said compound of Formula (I) or N-oxide thereof: 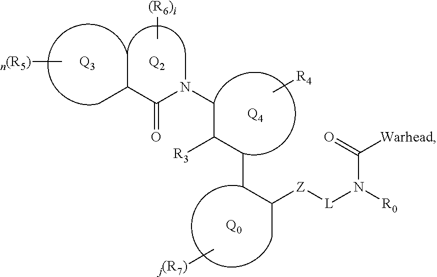
wherein
Q₀ is a 5-9 membered aryl or heteroaryl;
Q₂ is a 5-7 membered heterocycloalkyl;
Q₃ is a 5-membered heteroaryl or phenyl;
Q₄ is a 6-membered heteroaryl;
Z is absent, O, (CH₂)ₚO, O(CH₂)ₚO, N(H), (CH₂)ₚ, S, C(O), S(O₂), OC(O), C(O)O, OSO₂, S(O₂)O, C(O)S, SC(O), C(O)C(O), C(O)N(H), N(H)C(O), S(O₂)N(H), N(H)S(O₂), OC(O)O, OC(O)S, OC(O)N(H), N(H)C(O)O, N(H)C(O)S, N(H)C(O)N(H), (CH₂)ₚN(H)(CH₂)_{q}, (CH₂)ₚO(CH₂)_{q}, (CH₂)ₚN(H)C(O)(CH₂)_{q}, (CH₂)ₚC(O)N(H)(CH₂)_{q}, OC(O)N(H)(CH₂)ₚ₊₁N(H)(CH₂)_{q}, a bivalent alkenyl group, or a bivalent alkynyl group;
L is -L₁-L₂-;
L₁ is a bond, alkyl, alkenyl, alkynyl, cycloalkyl, cycloalkenyl, heterocycloalkyl, heterocycloalkenyl, aryl, or heteroaryl, in which said alkenyl, alkynyl, cycloalkyl, cycloalkenyl, heterocycloalkyl, heterocycloalkenyl, aryl, or heteroaryl is optionally subsitiuted with one or more Rₐ;
L₂ is a bond, or an alkyl in which one or more -Lᵢ- are optionally inserted between any two adjacent carbon atoms;
-Lᵢ- is —N(Rₐ)—, —O—, —S—, —C(O)—, —S(O₂)—, —OC(O)—, —C(O)O—, —OSO₂—, —S(O₂)O—, —C(O)S—, —SC(O)—, —C(O)C(O)—, —C(O)N(Rₐ)—, —N(Rₐ)C(O)—, —S(O₂)N(Rₐ)—, —N(Rₐ)S(O₂)—, —OC(O)O—, —OC(O)S—, —OC(O)N(Rₐ)—, —N(Rₐ)C(O)O—, —N(Rₐ)C(O)S—, —N(Rₐ)C(O)N(Rₐ)—, a bivalent alkenyl group, a bivalent alkynyl group, a bivalent cycloalkyl group, a bivalent heterocycloalkyl group, a bivalent aryl group, a bivalent heteroaryl group;
Warhead is 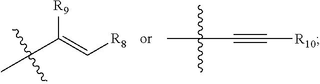
each of R₀, R₅, R₆, R₇, and R₈, independently, is H, D, alkyl, spiroalkyl, alkenyl, alkynyl, cycloalkyl, cycloalkenyl, heterocycloalkyl, spiroheterocycloalkyl, heterocycloalkenyl, aryl, heteroaryl, halo, nitro, oxo, cyano, ORₐ, SRₐ, alkyl-Rₐ, NH(CH₂)ₚRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c}, -alkyl-P(O)R_{b}R_{c}, —S(O)(═N(R_{b}))R_{c}, —N═S(O)R_{b}R_{c}, ═NR_{b}, SO₂N(R_{b})R_{c}, or N(R_{b})SO₂R_{c}, in which said cycloalkyl, cycloalkenyl, heterocycloalkyl, heterocycloalkenyl, aryl, heteroaryl is optionally subsitiuted with one or more R_{d};
R₃ is H, halo, alkyl, haloalkyl, or hydroxyalkyl;
R₄ is H, halo, or low alkyl;
R₉ is H, or cyano;
R₁₀ is H, alkyl, or haloalkyl;
R₀ groups and L, taken together with the atom to which they are attached, may optionally form a cycloalkyl, heterocycloalkyl, aryl, or heteroaryl optionally subsitiuted with one or more R_{d};
two of R₅ groups, taken together with the atom to which they are attached, may optionally form a cycloalkyl, heterocycloalkyl, aryl, or heteroaryl optionally subsitiuted with one or more R_{d};
two of R₆ groups, taken together with the atom to which they are attached, may optionally form a cycloalkyl or heterocycloalkyl optionally subsitiuted with one or more R_{d};
two of R₇ groups, taken together with the atom to which they are attached, may optionally form a cycloalkyl, heterocycloalkyl, aryl, or heteroaryl optionally subsitiuted with one or more R_{d};
Rₐ, R_{b}, R_{c} and R_{d}, independently, is H, D, alkyl, spiroalkyl, alkenyl, alkynyl, halo, cyano, amine, nitro, hydroxy, ═O, —P(O)R_{b}R_{c}, -alkyl-P(O)R_{b}R_{c}, —S(O)(═N(R_{b}))R_{c}, —N═S(O)R_{b}R_{c}, ═NR_{b}, C(O)NHOH, C(O) OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl, in which said alkyl, cycloalkyl, cycloalkenyl, heterocycloalkyl, heterocycloalkenyl, aryl, heteroaryl is optionally subsitiuted with one or more Rₑ;
Rₑ is H, D, alkyl, spiroalkyl, alkenyl, alkynyl, halo, cyano, amine, nitro, hydroxy, ═O, C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl;
two of R_{d} groups, taken together with the atom to which they are attached, may optionally form a cycloalkyl or heterocycloalkyl optionally subsitiuted with one or more Rₑ; and
each of i, j, n, p, and q, independently, is 0, 1, 2, 3, or 4.

2. The compound according to claim 1 or an N-oxide thereof, or a pharmaceutically acceptable salt, solvate, polymorph, tautomer, stereoisomer, an isotopic form, or a prodrug thereof, wherein the compound is represented by Formula (II): 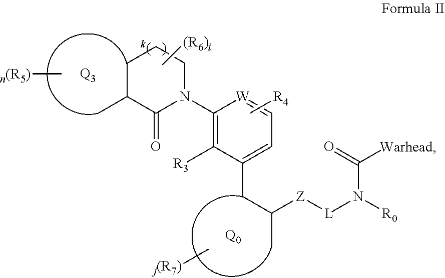
wherein
W is CH or N; and
k is 0, 1, or 2;

3. The compound according to claim 2 or an N-oxide thereof, or a pharmaceutically acceptable salt, solvate, polymorph, tautomer, stereoisomer, an isotopic form, or a prodrug thereof, wherein the compound is represented by Formula (III): 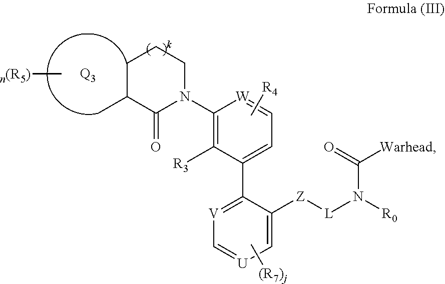
wherein
each of V, U, independently is C(Rₐ) or N.

4. The compound according to claim 3 or an N-oxide thereof, or a pharmaceutically acceptable salt, solvate, polymorph, tautomer, stereoisomer, an isotopic form, or a prodrug thereof, wherein the compound is represented by Formula (IV) wherein: 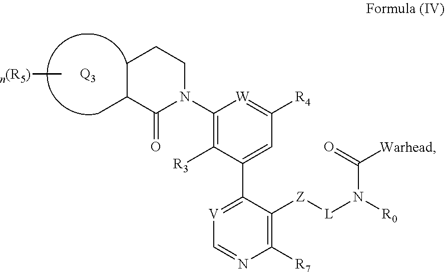
wherein
R₄ is H or F;
Z is O, or 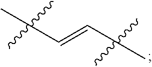 [SMILES: */C=C/*]
and
Warhead is

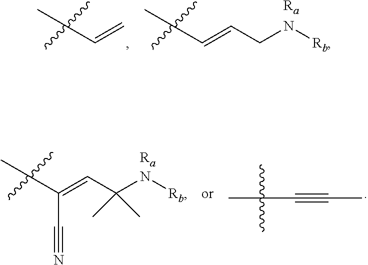

5. The compound according to claim 4 or an N-oxide thereof, or a pharmaceutically acceptable salt, solvate, polymorph, tautomer, stereoisomer, an isotopic form, or a prodrug thereof, wherein the compound is represented by Formula (V) wherein:

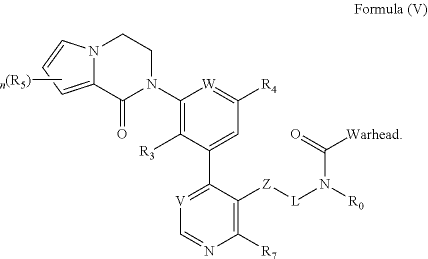

6. The compound according to claim 1 or an N-oxide thereof, or a pharmaceutically acceptable salt, solvate, polymorph, tautomer, stereoisomer, an isotopic form, or a prodrug thereof, wherein the compound is (S)-2-(3-(5-((1-acryloylpyrrolidin-2-yl)methoxy)-6-aminopyrimidin-4-yl)-5-fluoro-2-methylphenyl)-7,7-dimethyl-3,4,7,8-tetrahydro-2H-cyclopenta[4,5]pyrrolo[1,2-a]pyrazin-1(6H)-one.

7. A pharmaceutical composition comprising a compound of Formula (I) or an N-oxide thereof as defined in claims 1, or a pharmaceutically acceptable salt, solvate, polymorph, tautomer, stereoisomer, an isotopic form, or a prodrug of said compound of Formula (I) or an N-oxide thereof, and a pharmaceutically acceptable diluent or carrier.

8. A method of treating a neoplastic disease, autoimmune disease, and inflammatory disorder, comprising administering to a subject in need thereof an effective amount of a compound of Formula (I) or an N-oxide thereof as defined in claims 1, or a pharmaceutically acceptable salt, solvate, polymorph, tautomer, stereoisomer, an isotopic form, or a prodrug of said compound of Formula (I) or an N-oxide thereof.

9. A compound of Formula (A), or an N-oxide thereof, or a pharmaceutically acceptable salt, solvate, polymorph, tautomer, stereoisomer, an isotopic form, or a prodrug of said compound of Formula (A) or N-oxide thereof: 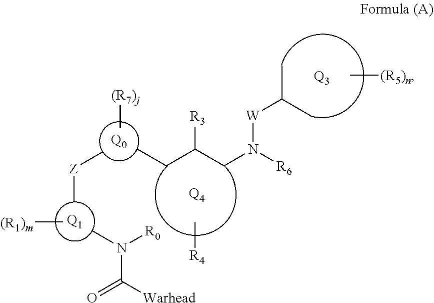
wherein
each of Q₀, Q₁, and Q₃, independently, is a 5-9 membered aryl or 5-9 membered heteroaryl;
Q₄ is a 6-membered heteroaryl;
W is —CH₂—, —C(O)—, or —S(O₂)—;
Z is NH, S, or O;
Warhead is 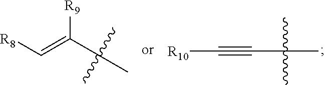
each of R₀, R₁, R₅, R₆, R₇, R₈, R₉, and R₁₀, independently, is H, D, alkyl, spiroalkyl, alkenyl, alkynyl, cycloalkyl, cycloalkenyl, heterocycloalkyl, spiroheterocycloalkyl, heterocycloalkenyl, aryl, heteroaryl, halo, nitro, oxo, cyano, ORₐ, SRₐ, alkyl-Rₐ, NH(CH₂)ₚRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)Rₑ, —P(O)R_{b}R_{c}, -alkyl-P(O)R_{b}R_{c}, —S(O)(═N(R_{b}))R_{c}, —N═S(O)R_{b}R_{c}, ═NR_{b}, SO₂N(R_{b})R_{c}, or N(R_{b})SO₂R_{c}, in which said cycloalkyl, cycloalkenyl, heterocycloalkyl, heterocycloalkenyl, aryl, heteroaryl is optionally subsitiuted with one or more R_{d};
R₃ is H, halo, alkyl, haloalkyl, or hydroxyalkyl;
R₄ is H, halo, or low alkyl;
R₀ and R₁ groups, taken together with the atom to which they are attached, may optionally form a cycloalkyl or heterocycloalkyl optionally subsitiuted with one or more R_{d};
two of R₁ groups, taken together with the atom to which they are attached, may optionally form a cycloalkyl or heterocycloalkyl optionally subsitiuted with one or more R_{d};
two of R₅ groups, taken together with the atom to which they are attached, may optionally form a cycloalkyl, heterocycloalkyl, aryl, or heteroaryl optionally subsitiuted with one or more R_{d};
two of R₆ groups, taken together with the atom to which they are attached, may optionally form a cycloalkyl or heterocycloalkyl optionally subsitiuted with one or more R_{d};
two of R₇ groups, taken together with the atom to which they are attached, may optionally form a cycloalkyl, heterocycloalkyl, aryl, or heteroaryl optionally subsitiuted with one or more R_{d};
Rₐ, R_{b}, R_{c} and R_{d}, independently, is H, D, alkyl, spiroalkyl, alkenyl, alkynyl, halo, cyano, amine, nitro, hydroxy, ═O, —P(O)R_{b}R_{c}, -alkyl-P(O)R_{b}R_{c}, —S(O)(═N(R_{b}))Rₑ, —N═S(O)R_{b}R_{c}, ═NR_{b}, C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl, in which said alkyl, cycloalkyl, cycloalkenyl, heterocycloalkyl, heterocycloalkenyl, aryl, heteroaryl is optionally subsitiuted with one or more Rₑ;
Rₑ is H, D, alkyl, spiroalkyl, alkenyl, alkynyl, halo, cyano, amine, nitro, hydroxy, ═O, C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl;
two of R_{d} groups, taken together with the atom to which they are attached, may optionally form a cycloalkyl or heterocycloalkyl optionally subsitiuted with one or more R^{e}; and
each of i, j, m, n, p, and q, independently, is 0, 1, 2, 3, or 4.

10. The compound according to claim 9 or an N-oxide thereof, or a pharmaceutically acceptable salt, solvate, polymorph, tautomer, stereoisomer, an isotopic form, or a prodrug thereof, wherein the compound is represented by Formula (B):

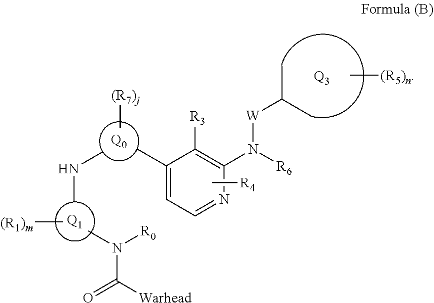

11. The compound according to claim 10 or an N-oxide thereof, or a pharmaceutically acceptable salt, solvate, polymorph, tautomer, stereoisomer, an isotopic form, or a prodrug thereof, wherein the compound is represented by Formula (C):

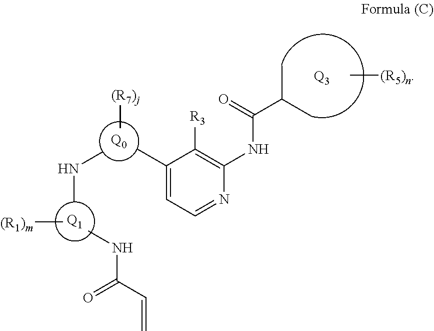

12. The compound according to claim 11 or an N-oxide thereof, or a pharmaceutically acceptable salt, solvate, polymorph, tautomer, stereoisomer, an isotopic form, or a prodrug thereof, wherein the compound is represented by Formula (D) wherein:

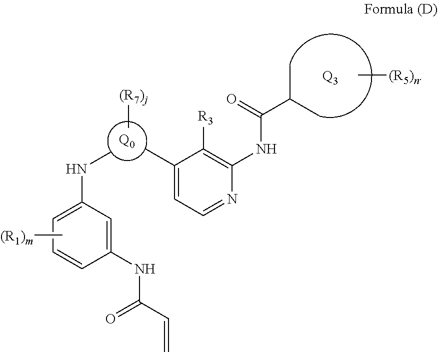

13. The compound according to claim 12 or an N-oxide thereof, or a pharmaceutically acceptable salt, solvate, polymorph, tautomer, stereoisomer, an isotopic form, or a prodrug thereof, wherein the compound is represented by Formula (E) wherein:

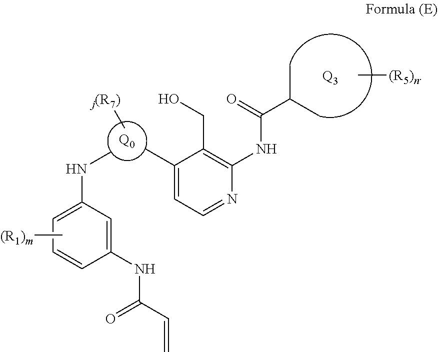

14. The compound according to claim 9 or an N-oxide thereof, or a pharmaceutically acceptable salt, solvate, polymorph, tautomer, stereoisomer, an isotopic form, or a prodrug thereof, wherein the compound is
(S)-N-(4-(6-((3-acrylamido-4-(2-methyl-4-(tetrahydro-2H-pyran-4-yl)piperazin-1-yl)phenyl)amino)-4-methyl-5-oxo-4,5-dihydropyrazin-2-yl)-3-(hydroxymethyl)pyridin-2-yl)-4-(tert-butyl)benzamide,
(S)-N-(4-(6-((3-acrylamido-4-(2-methyl-4-(tetrahydro-2H-pyran-4-yl)piperazin-1-yl)phenyl)amino)-4-methyl-5-oxo-4,5-dihydropyrazin-2-yl)-3-(hydroxymethyl)pyridin-2-yl)-4-(tert-butyl)-2-fluorobenzamide,
(S)-N-(4-(6-((3-acrylamido-4-(2-methyl-4-(tetrahydro-2H-pyran-4-yl)piperazin-1-yl)phenyl)amino)-4-methyl-5-oxo-4,5-dihydropyrazin-2-yl)-3-(hydroxymethyl)pyridin-2-yl)-4-cyclopropyl-2-fluorobenzamide,
(S)-N-(4-(6-((3-acrylamido-4-(2-methyl-4-(tetrahydro-2H-pyran-4-yl)piperazin-1-yl)phenyl)amino)-4-methyl-5-oxo-4,5-dihydropyrazin-2-yl)-3-(hydroxymethyl)pyridin-2-yl)-4-cyclopropylbenzamide,
(S)-N-(4-(6-((3-acrylamido-4-(2-methyl-4-(tetrahydro-2H-pyran-4-yl)piperazin-1-yl)phenyl)amino)-4-methyl-5-oxo-4,5-dihydropyrazin-2-yl)-3-(hydroxymethyl)pyridin-2-yl)-1,5,5-trimethyl-1,4,5,6-tetrahydrocyclopenta[b]pyrrole-2-carboxamide,
(S)-N-(4-(6-((3-acrylamido-4-(2-methyl-4-(tetrahydro-2H-pyran-4-yl)piperazin-1-yl)phenyl)amino)-4-methyl-5-oxo-4,5-dihydropyrazin-2-yl)-3-(hydroxymethyl)pyridin-2-yl)-5,5-dimethyl-1,4,5,6-tetrahydrocyclopenta[b]pyrrole-2-carboxamide,
(S)-N-(4-(6-((3-acrylamido-4-(2-methyl-4-(tetrahydro-2H-pyran-4-yl)piperazin-1-yl)phenyl)amino)-4-methyl-5-oxo-4,5-dihydropyrazin-2-yl)-3-(hydroxymethyl)pyridin-2-yl)-5-(tert-butyl)thiophene-2-carboxamide,
(S)-N-(4-(6-((3-acrylamido-4-(2-methyl-4-(tetrahydro-2H-pyran-4-yl)piperazin-1-yl)phenyl)amino)-4-methyl-5-oxo-4,5-dihydropyrazin-2-yl)-3-(hydroxymethyl)pyridin-2-yl)-4,5,6,7-tetrahydrobenzo[b]thiophene-2-carboxamide,
(S)-N-(6-((3-acrylamido-4-(2-methyl-4-(tetrahydro-2H-pyran-4-yl)piperazin-1-yl)phenyl)amino)-3′-(hydroxymethyl)[2,4′-bipyridin]-2′-yl)-4-(tert-butyl)-2-fluorobenzamide,
(S)-N-(4-(6-(3-acrylamido-4-(2-methyl-4-(tetrahydro-2H-pyran-4-yl)piperazin-1-yl)phenyl)amino)pyrazin-2-yl)-3-(hydroxymethyl)pyridin-2-yl)-4-(tert-butyl)-2-fluorobenzamide,
(S)-N-(4-(2-((3-acrylamido-4-(2-methyl-4-(tetrahydro-2H-pyran-4-yl)piperazin-1-yl)phenyl)amino)pyrimidin-4-yl)-3-(hydroxymethyl)pyridin-2-yl)-4-cyclopropyl-2-fluorobenzamide,
(S)-N-(4-(4-((3-acrylamido-4-(2-methyl-4-(tetrahydro-2H-pyran-4-yl)piperazin-1-yl)phenyl)amino)-1,3,5-triazin-2-yl)-3-(hydroxymethyl)pyridin-2-yl)-4-cyclopropyl-2-fluorobenzamide,
(S)-N-(4-(4-((3-acrylamido-4-(2-methyl-4-(tetrahydro-2H-pyran-4-yl)piperazin-1-yl)phenyl)amino)pyrimidin-2-yl)-3-(hydroxymethyl)pyridin-2-yl)-4-(tert-butyl)-2-fluorobenzamide,
(S)-N-(4-(4-((3-acrylamido-4-(2-methyl-4-(tetrahydro-2H-pyran-4-yl)piperazin-1-yl)phenyl)amino)-5-(trifluoromethyl)pyrimidin-2-yl)-3-(hydroxymethyl)pyridin-2-yl)-4-(tert-butyl)-2-fluorobenzamide,
(S)-N-(4-(4-((3-acrylamido-4-(2-methyl-4-(tetrahydro-2H-pyran-4-yl)piperazin-1-yl)phenyl)amino)-5-methoxypyrimidin-2-yl)-3-(hydroxymethyl)pyridin-2-yl)-4-(tert-butyl)-2-fluorobenzamide,
(S)-N-(4-(4-((3-acrylamido-4-(2-methyl-4-(tetrahydro-2H-pyran-4-yl)piperazin-1-yl)phenyl)amino)-5-isopropoxypyrimidin-2-yl)-3-(hydroxymethyl)pyridin-2-yl)-4-(tert-butyl)-2-fluorobenzamide,
(S)-N-(4-(4-((3-acrylamido-4-(2-methyl-4-(tetrahydro-2H-pyran-4-yl)piperazin-1-yl)phenyl)amino)-5-aminopyrimidin-2-yl)-3-(hydroxymethyl)pyridin-2-yl)-4-(tert-butyl)-2-fluorobenzamide,
(S)-N-(4-(4-((3-acrylamido-4-(2-methyl-4-(tetrahydro-2H-pyran-4-yl)piperazin-1-yl)phenyl)amino)-5-(methylamino)pyrimidin-2-yl)-3-(hydroxymethyl)pyridin-2-yl)-4-(tert-butyl)-2-fluorobenzamide,
(S)-3-((3-acrylamido-4-(2-methyl-4-(tetrahydro-2H-pyran-4-yl)piperazin-1-yl)phenyl)amino)-1-(2-(4-cyclopropyl-2-fluorobenzamido)-3-(hydroxymethyl)pyridin-4-yl)-1H-pyrazole-4-carboxamide,
(S)-N-(4-(3-((3-acrylamido-4-(2-methyl-4-(tetrahydro-2H-pyran-4-yl)piperazin-1-yl)phenyl)amino)-4-oxo-4,5,6,7-tetrahydro-1H-pyrazolo[4,3-c]pyridin-1-yl)-3-(hydroxymethyl)pyridin-2-yl)-4-cyclopropyl-2-fluorobenzamide,
(S)-N-(4-(8-((3-acrylamido-4-(2-methyl-4-(tetrahydro-2H-pyran-4-yl)piperazin-1-yl)phenyl)amino)imidazo[1,2-a]pyrazin-6-yl)-3-(hydroxymethyl)pyridin-2-yl)-4-(tert-butyl)-2-fluorobenzamide,
(S)-N-(4-(6-((3-acrylamido-4-(2-methyl-4-(oxetan-3-yl)piperazin-1-yl)phenyl)amino)-4-methyl-5-oxo-4,5-dihydropyrazin-2-yl)-3-(hydroxymethyl)pyridin-2-yl)-4-cyclopropyl-2-fluorobenzamide,
(R)-N-(4-(6-((3-acrylamido-4-(4-(oxetan-3-yl)-2-(trifluoromethyl)piperazin-1-yl)phenyl)amino)-4-methyl-5-oxo-4,5-dihydropyrazin-2-yl)-3-(hydroxymethyl)pyridin-2-yl)-4-cyclopropyl-2-fluorobenzamide,
(S)-N-(4-(6-((3-acrylamido-4-(2-methyl-4-(tetrahydro-2H-pyran-4-yl)piperazin-1-yl)phenyl)amino)-4-methyl-5-oxo-4,5-dihydropyrazin-2-yl)-3-methylpyridin-2-yl)-4-cyclopropyl-2-fluorobenzamide.

15. A pharmaceutical composition comprising a compound of Formula (A) or an N-oxide thereof as defined in claims 9, or a pharmaceutically acceptable salt, solvate, polymorph, tautomer, stereoisomer, an isotopic form, or a prodrug of said compound of Formula (A) or an N-oxide thereof, and a pharmaceutically acceptable diluent or carrier.

16. A method of treating a neoplastic disease, autoimmune disease, and inflammatory disorder, comprising administering to a subject in need thereof an effective amount of a compound of Formula (A) or an N-oxide thereof as defined in claims 9, or a pharmaceutically acceptable salt, solvate, polymorph, tautomer, stereoisomer, an isotopic form, or a prodrug of said compound of Formula (A) or an N-oxide thereof.
